(19) United States
(12) Patent Application Publication
(10) Pub. No.: US 2026/0062699 A1
(43) Pub. Date: Mar. 5, 2026

(54) METHODS AND COMPOSITIONS FOR MODULATING SPLICING AT ALTERNATIVE SPLICE SITES

(71) Applicant: REMIX THERAPEUTICS INC., Watertown, MA (US)

(72) Inventors: Anant A. Agrawal, Waltham, MA (US); Frederic Vaillancourt, Newton, MA (US); Zaven Kaprielian, Rye, NY (US); Peter Smith, Arlington, MA (US); Alexander S. Harding, Boston, MA (US); Christopher Richer Neil, Bedford, MA (US); Maria Sarah Alexis, Boston, MA (US)

(21) Appl. No.: 19/103,514

(22) PCT Filed: Aug. 11, 2023
(86) PCT No.: PCT/US2023/030113

§ 371 Date: Feb. 12, 2025

Related U.S. Application Data
(60) Provisional application No. 63/397,661, filed on Aug. 12, 2022.

(51) Int. Cl. C12N 15/113; A61P 25/28

(52) U.S. Cl. CPC C12N 15/113; A61P 25/28; C12N 2310/11; C12N 2310/315; C12N 2310/321; C12N 2310/3231; C12N 2320/33

(57) ABSTRACT

The present disclosure features bifunctional oligonucleotides and related compositions that, inter alia, modulate nucleic CA acid splicing, e.g., splicing of a pre-mRNA, as well as methods of use thereof.

### CLAIM OF PRIORITY

This application claims priority to U.S. Patent Application No. 63/397,661, filed Aug. 12, 2022; the entire contents of the application is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns are often mapped to changes in splice site signals or sequence motifs and regulatory splicing factors (Faustino and Cooper (2003) Genes Dev 17(4):419-437). As such, there is a need for novel compositions and methods for targeting alternative splicing pathways to provide useful treatment modalities.

### SUMMARY

The present invention relates to oligonucleotide compounds with a multipartite (e.g., bipartite) architecture useful, e.g., for targeting an exonic element (e.g., alternative splice sites) within certain genes, as well as compositions and related methods thereof. In one aspect, the present disclosure features a bifunctional oligonucleotide capable of (i) binding to a target sequence (e.g., an RNA, e.g., a pre-mRNA or mRNA) comprising an exonic element, such as an alternative splice site; and (ii) recruiting a spliceosome component. In an embodiment, the bifunctional oligonucleotide is capable of modulating splicing of a target sequence, e.g., at an alternative splice site. In an embodiment, the bifunctional oligonucleotide is capable of modulating the production or level of a transcription product (e.g., an RNA, e.g., a pre-mRNA, or mRNA) or a target protein. In an embodiment, the target sequence contains repeated trinucleotides (e.g., more than 2, 5, 8, 10, 12, 14, 16, 18, 20, 22, 24, 26, 28, 30, 32, 34, 36, 38, 40, 42, 44, 46, 48, 50, 52, 54, 56, 60, or more repeated trinucleotides). In an embodiment, the target sequence is present within a mutant gene, e.g., a gene comprising at least one mutation, e.g., one repeated trinucleotide sequence. In an embodiment, the target sequence is present within a gene associated with a disease, disorder, or condition, such as a neurological disease or disorder, e.g., Huntington's disease (HD).

In an embodiment, the bifunctional oligonucleotide comprises: (i) an alternative splice site targeting sequence and (ii) a spliceosome targeting sequence. In an embodiment, the alternative splice site targeting sequence binds to a target sequence comprising an alternative splice site. In an embodiment, the alternative splice site targeting sequence binds directly to the alternative splice site. In an embodiment, the alternative splice site targeting sequence binds to a region in the target sequence that is 5′ to the alternative splice site. In an embodiment, the alternative splice site targeting sequence binds to a region in the target sequence that is 3′ to the alternative splice site. In an embodiment, the alternative splice site targeting sequence is a 5′ splice site sequence. In an embodiment, the alternative splice site targeting sequence is present at the 5′ or 3′ region of the bifunctional oligonucleotide. In an embodiment, the alternative splice site targeting sequence is present at the 5′ region of the bifunctional oligonucleotide. In an embodiment, the alternative splice site targeting sequence is present at the 3′ region of the bifunctional oligonucleotide.

In an embodiment, the alternative splice site targeting sequence is between 5 and 50 nucleotides in length (e.g., 5 to 45 nucleotides, 5 to 40 nucleotides, 5 to 35 nucleotides, 5 to 30 nucleotides, or 5 to 25 nucleotides in length). In an embodiment, the alternative splice site targeting sequence is between 5 and 50 nucleotides in length (e.g., 10 to 50 nucleotides, 15 to 50 nucleotides, 20 to 50 nucleotides, or 25 to 50 nucleotides in length).

In an embodiment, the spliceosome targeting sequence is between 5 and 50 nucleotides in length (e.g., 5 to 45 nucleotides, 5 to 40 nucleotides, 5 to 35 nucleotides, 5 to 30 nucleotides, or 5 to 25 nucleotides in length). In an embodiment, the spliceosome targeting sequence is between 5 and 25 nucleotides in length (e.g., 10 to 25 nucleotides or 15 to 25 nucleotides). In an embodiment, the spliceosome targeting sequence is present at the 5′ or 3′ region of the bifunctional oligonucleotide. In an embodiment, the spliceosome targeting sequence is present at the 5′ region of the bifunctional oligonucleotide. In an embodiment, the spliceosome targeting sequence is present at the 3′ region of the bifunctional oligonucleotide.

In an embodiment, the bifunctional oligonucleotide comprises a chemical modification, e.g., a non-naturally occurring modification. In an embodiment, the chemical modification comprises a nucleobase modification, a sugar (e.g., ribose) modification, an internucleotide modification, or a terminal modification. In an embodiment, the non-naturally occurring modification is a sugar (e.g., ribose) modification. In an embodiment, the non-naturally occurring modification is 2′-ribose modification, e.g., a 2′-O-alkyl (e.g., 2′-OMe), 2′-halo (e.g., 2′-F, 2′-Cl, or 2′-Br), 2′-methoxyethyl (2′-MOE), or 2′-deoxy modification. In an embodiment, the non-naturally occurring modification is a locked nucleic acid. In an embodiment, the non-naturally occurring modification is an internucleotide modification, e.g., a phosphorothioate modification. In an embodiment, the bifunctional oligonucleotide comprises a plurality of chemical modifications, e.g., at least 2, 3, 4, 5, 6, 7, 8, 9, 10, 11, 12, 13, 14, 15, 16, 17, 18, 19, 20, 21, 22, 23, 24, 25, 26, 27. 28, 29, 30, 31, 32, 33, 34, 35, 36, 37, 38, 39, 40, or more chemical modifications. In an embodiment, the bifunctional oligonucleotide comprises between 2 and 50 chemical modifications, e.g., between 5 and 45 chemical modifications, between 10 and 40 chemical modifications, between 15 and 35 chemical modifications, or between 20 and 30 chemical modifications. In an embodiment, at least 10%, 20%, 30%, 40%, 50%, 60%, 70%, 80%, 90%, 95%, or more nucleotides within the bifunctional oligonucleotide comprise a chemical modification. In an embodiment, every nucleotide within the bifunctional oligonucleotide comprises a chemical modification. In an embodiment, the bifunctional oligonucleotide does not comprise a chemical modification.

In another aspect, the present disclosure provides methods for preventing and/or treating a disease, disorder, or condition in a subject or cell by administering a bifunctional oligonucleotide described herein, or a pharmaceutically acceptable salt thereof, or related compositions, to the subject or cell. In some embodiments, the disease or disorder entails unwanted or aberrant splicing. In some embodiments, the disease or disorder is a repeat expansion disease. In some embodiments, the disease or disorder is a neurological disease or disorder. In some embodiments, the disease or disorder comprises Huntington's disease, Huntington's disease-like 2, holoprosencephaly 5, spinocerebellar ataxia type 1, spinocerebellar ataxia type 2, spinocerebellar ataxia type 3, spinocerebellar ataxia type 7, spinocerebellar ataxia type 8, spinocerebellar ataxia type 17, myotonic dystrophy type 1, oculopharyngodistal myopathy 2, oculopharyngodistal myopathy with leukoencephalopathy, X-linked intellectual disability, dentatorubral-pallidoluysian atrophy, spinal and bulbar atrophy, cleidocranial dysplasia, synpolydactyly 1, glutaminase deficiency, Jacobsen syndrome, fragile X syndrome, fragile X-associated primary ovarian insufficiency, fragile X-associated tremor/ataxia syndrome, X-linked hypopituitarism, or congenital central hypoventilation syndrome. In some embodiments, the disease or disorder is Huntington's disease.

In another aspect, the present disclosure provides methods of down-regulating the expression of (e.g., the level of or the rate of production of) a target nucleic acid (e.g., RNA) or target protein with a bifunctional oligonucleotide described herein, or a pharmaceutically acceptable salt thereof, or related compositions. In another aspect, the present disclosure provides methods of up-regulating the expression of (e.g., the level of or the rate of production of) a target nucleic acid (e.g., RNA) or target protein with a bifunctional oligonucleotide described herein, or a pharmaceutically acceptable salt thereof, or related compositions. In another aspect, the present disclosure provides methods of altering the isoform of a target nucleic acid (e.g., RNA) or target protein with a bifunctional oligonucleotide described herein, or a pharmaceutically acceptable salt thereof, or related compositions. Another aspect of the disclosure relates to methods of inhibiting the activity of a target nucleic acid (e.g., RNA) or target protein in a biological sample or subject. In some embodiments, administration of a bifunctional oligonucleotide to a biological sample, a cell, or a subject comprises inhibition of cell growth or induction of cell death.

In another aspect, the present disclosure features a method of modulating the production or level of a transcription product in a cell or subject comprising an exonic element (e.g., an alternative splice site within or near a trinucleotide expansion, e.g., a [CAG]ₙ site) in a subject or cell, wherein (i) the exonic element is flanked by a proximal splice site and a distal splice site, and (ii) the proximal splice site and distal splice sites are both 5′ splice sites or are both 3′ splice sites; comprising contacting said cell or subject with a bifunctional oligonucleotide capable of promoting splicing at (a) the distal 5′ splice site to (a-i) decrease the production or level of a transcription product comprising the exonic element or (a-ii) increase the production or level of a transcription product lacking the exonic element; or (b) the proximal 3′ splice site to (b-i) increase the production or level of a transcription product comprising the exonic element or (b-ii) decrease the production or level of a transcription product lacking the exonic element; thereby modulating the production or level of a transcription product comprising an exonic element (e.g., an alternative splice site within or near a trinucleotide expansion, e.g., a [CAG]ₙ site). In an embodiment, the method comprises (a-i) or (a-ii). In an embodiment, the method comprises (a-i). In an embodiment, the method comprises (b-i) or (b-ii). In an embodiment, the method comprises (b-i).

In an embodiment, the distal 5′ splice site is a non-canonical 5′ splice site (e.g., an alternative 5′ splice site). In an embodiment, the proximal 5′ splice site is a canonical 5′ splice site. In an embodiment, the distal 5′ splice site is a non-canonical 5′ splice site (e.g., an alternative 5′ splice site) and the exonic element comprises a canonical 5′ spice site. In an embodiment, the production or level of a transcription product produced by splicing at the distal 5′ splice site is increased by at least 1%, 5%, 10% 15%, 20%, 25%, 30%, 40%, or 50%, e.g., in comparison to a reference standard (e.g., the transcription product produced by splicing at a proximal 5′ splice site, wild type transcription product, or mutant transcription product).

In another aspect, the present disclosure provides compositions for use in preventing and/or treating a disease, disorder, or condition in a subject by administering a bifunctional oligonucleotide described herein, or a pharmaceutically acceptable salt thereof, or related compositions. In some embodiments, the disease or disorder entails unwanted or aberrant splicing. In some embodiments, the disease or disorder is a repeat expansion disease. In some embodiments, the disease or disorder is a neurological disease or disorder. In some embodiments, the disease or disorder comprises Huntington's disease, Huntington's disease-like 2, holoprosencephaly 5, spinocerebellar ataxia type 1, spinocerebellar ataxia type 2, spinocerebellar ataxia type 3, spinocerebellar ataxia type 7, spinocerebellar ataxia type 8, spinocerebellar ataxia type 17, myotonic dystrophy type 1, oculopharyngodistal myopathy 2, oculopharyngodistal myopathy with leukoencephalopathy, X-linked intellectual disability, dentatorubral-pallidoluysian atrophy, spinal and bulbar atrophy, cleidocranial dysplasia, synpolydactyly 1, glutaminase deficiency, Jacobsen syndrome, fragile X syndrome, fragile X-associated primary ovarian insufficiency, fragile X-associated tremor/ataxia syndrome, X-linked hypopituitarism, or congenital central hypoventilation syndrome. In some embodiments, the disease or disorder is Huntington's disease.

In another aspect, the present disclosure features kits comprising a container with a bifunctional oligonucleotide described herein, or a pharmaceutically acceptable salt thereof, or related compositions. In certain embodiments, the kits described herein further include instructions for administering the bifunctional oligonucleotide or the pharmaceutically acceptable salt, solvate, hydrate, tautomer, stereoisomer thereof, or the pharmaceutical composition thereof.

The details of one or more embodiments of the invention are set forth herein. Other features, objects, and advantages of the invention will be apparent from the Detailed Description, the Examples, and the Claims.

### BRIEF DESCRIPTION OF DRAWINGS

FIGS. 1A-1B are schematics demonstrating exemplary orientations for the bifunctional oligonucleotide. In these renderings, the bifunctional oligonucleotide comprises a bipartite architecture, with both an alternative splice site targeting sequence (dark gray) and a spliceosome targeting sequence (light gray). The U1 snRNP complex is depicted as a gray circle binding to the bifunctional oligonucleotides.

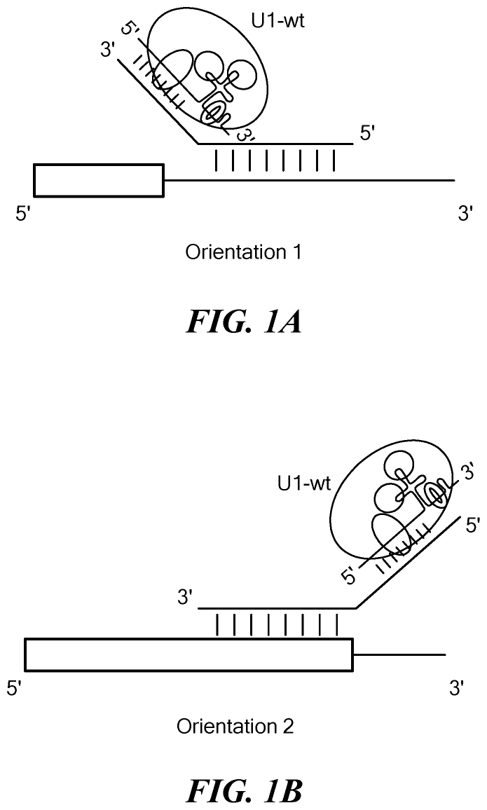

FIG. 2 is a bar graph demonstrating the beneficial effect of chemical modifications, specifically the locked nucleic acids (LNAs), on the bifunctional oligonucleotides, for an exemplary target gene.

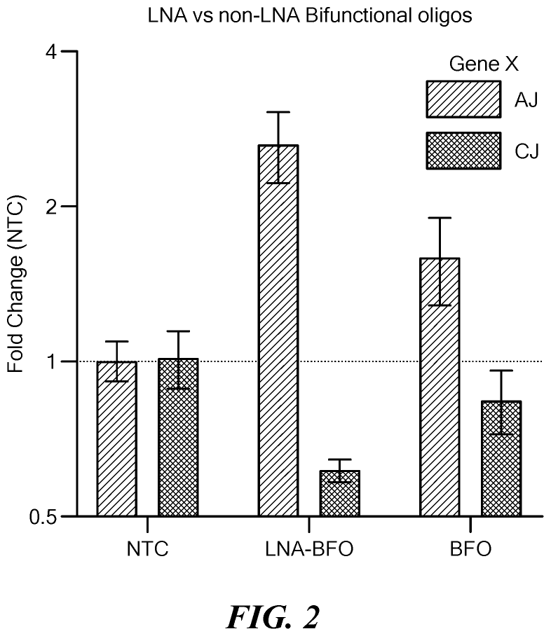

### DETAILED DESCRIPTION

The present invention relates to bifunctional oligonucleotides and related compositions and methods, useful for modulating splicing at a target sequence. Exemplary target sequences include target sequences comprising an alternative splice site and can be correlated, for example, with a repeat expansion disease (e.g., Huntington's disease).

### Definitions

Unless defined otherwise, all technical and scientific terms used herein have the same meaning as commonly understood by one of ordinary skill in the art to which the invention pertains.

As used herein, the terms “a” and “an” refer to one or to more than one (i.e., to at least one) of the grammatical object of the article. By way of example, “an element” means one element or more than one element.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “acquire” or “acquiring,” refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., a fluorimeter to acquire fluorescence data.

The term “alternative splice site,” as used herein, refers to a non-canonical splice site, e.g., within a specific pre-mRNA sequence. Splicing at an alternative splice site by the spliceosome may result in a difference in the sequence of the target, thus contributing to the diversity of the proteome. An alternative splice site may be present 5′ or 3′ to the canonical splice site in pre-mRNA sequence.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, and 5.18% without consideration of the number of significant figures.)

A “distal splice site,” as used herein, refers to a splice site wherein the exonic element is disposed between the distal splice site and the cognate intron.

The term “exonic element,” as used herein, refers to a nucleotide sequence within an exon comprising a sequence of interest, e.g., a repeated trinucleotide sequence, such as [CAG]ₙ when n is greater than 5, e.g., greater than 8, 10, 12, 16, 18, or more.

The term “expression” refers to the transcription and/or translation of a particular nucleotide sequence driven by a promoter.

The term “oligonucleotide,” as used herein, is defined as it is generally understood by the skilled person as a molecule comprising two or more covalently linked nucleosides. Such covalently bound nucleosides can be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide can, for example, be chemically synthesized, and be purified or isolated. The oligonucleotide is also intended to include (i) compounds that have one or more furanose moieties that are replaced by furanose derivatives or by any structure, cyclic or acyclic, that can be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that can be used as a point of covalent attachment for the base moiety. The oligonucleotide can comprise one or more alternative nucleosides or nucleotides (e.g., including those described herein). It is also understood that oligonucleotide includes compositions lacking a sugar moiety or nucleobase but are still capable of forming a pairing with or hybridizing to a target sequence.

The term “nucleic acid” refers to deoxyribonucleic acids (DNA) or ribonucleic acids (RNA) and polymers thereof, e.g., in either single- or double-stranded form. The term “nucleic acid” includes a gene, cDNA, pre-mRNA or an mRNA. In one embodiment, the nucleic acid molecule is synthetic (e.g., chemically synthesized) or recombinant. Unless specifically limited, the term encompasses nucleic acids containing natural and/or synthetic analogues or derivatives of natural nucleotides and/or non-natural internucleoside linkages, that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, an amount of a bifunctional oligonucleotide “effective to treat a disorder,” (e.g., a disorder described herein), “therapeutically effective amount,” “effective amount” or “effective course” refers to an amount of a bifunctional oligonucleotide which is effective, upon single or multiple dose administration(s) to a subject, in treating a subject, or in curing, alleviating, relieving or improving a subject with a disorder (e.g., a repeat expansion disease) beyond that expected in the absence of such treatment.

As used herein, the terms “prevent” or “preventing” as used in the context of a disorder or disease, refer to administration of an agent to a subject, e.g., the administration of a bifunctional oligonucleotide of the present disclosure to a subject, such that the onset of at least one symptom of the disorder or disease is delayed as compared to what would be seen in the absence of administration of said treatment.

A “proximal splice site,” as used herein, refers to a splice site disposed between the exonic element and the proximal splice site's cognate intron.

As used herein, the term “subject” is intended to include human and non-human animals. Exemplary human subjects include a human patient having a disorder, e.g., a disorder described herein, or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc.

As used herein, the terms “treat” or “treating” a subject having a disorder or disease refer to subjecting the subject to a regimen, e.g., the administration of a bifunctional oligonucleotide or pharmaceutically acceptable salt thereof, or a composition comprising a bifunctional oligonucleotide or pharmaceutically acceptable salt thereof, such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease. In some embodiments, treatment comprises prevention. In some embodiments, treatment does not comprise prevention.

Numerous ranges, e.g., ranges for the amount of a bifunctional oligonucleotide or a composition thereof administered per day, are provided herein. In some embodiments, the range includes both endpoints. In other embodiments, the range excludes one or both endpoints. By way of example, the range can exclude the lower endpoint. Thus, in such an embodiment, a range of 100 to 1000 mg/day, excluding the lower endpoint, would cover an amount greater than 100 that is less than or equal to 1000 mg/day.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 36 carbon atoms (“C₁-C₃₆ alkyl”). In some embodiments, an alkyl group has 1 to 32 carbon atoms (“C₁-C₃₂ alkyl”). In some embodiments, an alkyl group has 1 to 24 carbon atoms (“C₁-C₂₄ alkyl”). In some embodiments, an alkyl group has 1 to 18 carbon atoms (“C₁-C₁₈ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C_{L}-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₂₄ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tert-amyl (C₅), n-hexyl (C₆), octyl (C₅), nonyl (C₉), decyl (C₁₀), undecyl (C₁₁), dodecyl (or lauryl) (Ct2), tridecyl (C₁₃), tetradecyl (or myristyl) (C₁₄), pentadecyl (C₁₅), hexadecyl (or cetyl) (C₁₆), heptadecyl (C₁₇), octadecyl (or stearyl) (C₁₈), nonadecyl (C₁₉), eicosyl (or arachidyl) (C₂₀), henicosanyl (C₂₁), docosanyl (C₂₂), tricosanyl (C₂₃), and tetracosanyl (C₂₄). Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 36 carbon atoms, one or more carbon-carbon double bonds, and no triple bonds (“C₂-C₃₆ alkenyl”). In some embodiments, an alkenyl group has 2 to 32 carbon atoms (“C₂-C₃₂ alkenyl”). In some embodiments, an alkenyl group has 2 to 24 carbon atoms (“C₂-C₂₄ alkenyl”). In some embodiments, an alkenyl group has 2 to 18 carbon atoms (“C₂-C₁₅ alkenyl”).

In some embodiments, an alkenyl group has 2 to 12 carbon atoms (“C₂-C₁₂ alkenyl”). In some embodiments, an alkenyl group has 2 to 8 carbon atoms (“C₂-C₅ alkenyl”). In some embodiments, an alkenyl group has 2 to 7 carbon atoms (“C₂-C₇ alkenyl”). In some embodiments, an alkenyl group has 2 to 8 carbon atoms (“C₂-C₅ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). The one or more carbon double bonds can have cis or trans (or E or Z) geometry. Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₂₄ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₅), nonenyl (C₉), nonadienyl (C₉), decenyl (C₁₀), decadienyl (C₁₀), undecenyl (C₁₁), undecadienyl (C₁₁), dodecenyl (C₁₂), dodecadienyl (C₁₂), tridecenyl (C₁₃), tridecadienyl (C₁₃), tetradecenyl (C₁₄), tetradecadienyl (e.g., myristoleyl) (C₁₄), pentadecenyl (C₁₅), pentadecadienyl (C₁₅), hexadecenyl (e.g., palmitoleyl) (C₁₆), hexadecadienyl (C₁₆), heptadecenyl (C₁₇), heptadecadienyl (C₁₇), octadecenyl (e.g., oleyl) (C₁₈), octadecadienyl (e.g., linoleyl) (C₁₈), nonadecenyl (C₁₉), nonadecadienyl (C₁₉), eicosenyl (C₂₀), eicosadienyl (C₂₀), eicosatrienyl (C₂₀), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl.

As used herein, the term “alkynyl” refers to a radical of a straight-chain or branched hydrocarbon group having from 2 to 36 carbon atoms, one or more carbon-carbon triple bonds (“C₂-C₃₆ alkynyl”). In some embodiments, an alkynyl group has 2 to 32 carbon atoms (“C₂-C₃₂ alkynyl”). In some embodiments, an alkynyl group has 2 to 24 carbon atoms (“C₂-C₂₄ alkynyl”).

In some embodiments, an alkynyl group has 2 to 18 carbon atoms (“C₂-C₁₈ alkynyl”). In some embodiments, an alkynyl group has 2 to 12 carbon atoms (“C₂-C₁₂ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl).

Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the terms “heteroalkyl,” “heteroalkenyl,” and “heteroalkynyl,” refer to a non-cyclic stable straight or branched alkyl, alkenyl, or alkynyl chains, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl, heteroalkenyl, or heteroalkynyl group. Exemplary heteroalkyl, heteroalkenyl, and heteroalkynyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CHO—CH₃, —Si(CH₃)₃, —CH₂—CH₂—P(O)₂—CH₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. The term “alkenylene,” by itself or as part of another substituent, means, unless otherwise stated, a divalent radical derived from an alkene. An alkylene, alkenylene, alkynylene, or heteroalkylene group may be described as, e.g., a C₁-C₆-membered alkylene, C₁-C₆-membered alkenylene, C₁-C₆-membered alkynylene, or C₁-C₆-membered heteroalkylene, wherein the term “membered” refers to the non-hydrogen atoms within the moiety. In the case of heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′—and —R′C(O)₂—. Each instance of an alkylene, alkenylene, alkynylene, or heteroalkylene group may be independently optionally substituted, i.e., unsubstituted (an “unsubstituted alkylene”) or substituted (a “substituted heteroalkylene) with one or more substituents.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “cycloalkyl” refers to a radical of a non-aromatic cyclic hydrocarbon group having from 3 to 7 ring carbon atoms (“C₃-C₇ cycloalkyl”) and zero heteroatoms in the non-aromatic ring system. In some embodiments, a cycloalkyl group has 3 to 6 ring carbon atoms (“C₃-C₆ cycloalkyl”). In some embodiments, a cycloalkyl group has 5 to 7 ring carbon atoms (“C₅-C₇ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₇ cycloalkyl groups include, without limitation, the aforementioned C₃-C₆ cycloalkyl groups as well as cycloheptyl (C₇), cycloheptenyl (C₇), cycloheptadienyl (C₇), and cycloheptatrienyl (C₇), bicyclo[2.1.1]hexanyl (C₆), bicyclo[3.1.1]heptanyl (C₇), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

As used herein, the term “halo” refers to a fluorine, chlorine, bromine, or iodine radical (i.e., —F, —Cl, —Br, and —I, respectively).

As used herein, the term “heteroaryl,” refers to an aromatic heterocycle that comprises 1, 2, 3 or 4 heteroatoms selected, independently of the others, from nitrogen, sulfur and oxygen. As used herein, the term “heteroaryl” refers to a group that may be substituted or unsubstituted. A heteroaryl may be fused to one or two rings, such as a cycloalkyl, an aryl, or a heteroaryl ring. The point of attachment of a heteroaryl to a molecule may be on the heteroaryl, cycloalkyl, heterocycloalkyl or aryl ring, and the heteroaryl group may be attached through carbon or a heteroatom. A heteroaryl group can either be monocyclic (“monocyclic heteroaryl”) or a fused, bridged or spiro ring system such as a bicyclic system (“bicyclic heteroaryl”). Examples of heteroaryl groups include imidazolyl, furyl, pyrrolyl, thienyl, thiazolyl, isoxazolyl, isothiazolyl, thiadiazolyl, oxadiazolyl, pyridinyl, pyrimidyl, pyrazinyl, pyridazinyl, quinolyl, isoquinolinyl, indazolyl, benzoxazolyl, benzisooxazolyl, benzofuryl, benzothiazolyl, indolizinyl, imidazopyridinyl, pyrazolyl, triazolyl, oxazolyl, tetrazolyl, benzimidazolyl, benzoisothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidyl, pyrazolo[3,4]pyrimidyl or benzo(b)thienyl, each of which can be optionally substituted.

As used herein, the term “heterocyclyl” refers to non-aromatic ring system having ring carbon atoms and 1 to 4 ring heteroatoms, wherein each heteroatom is independently selected from nitrogen, oxygen, sulfur, boron, phosphorus, and silicon. As used herein, the term “heterocyclyl” refers to a group that may be substituted or unsubstituted. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. In certain embodiments, the heterocyclyl group is unsubstituted 3-10 membered heterocyclyl. In certain embodiments, the heterocyclyl group is substituted 3-10 membered heterocyclyl.

As used herein, the term “hydroxy” refers to the radical —OH.

The term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O— alkyl, or other modification.

The term “oligonucleotide,” as used herein, refers to a polymer of nucleotide or nucleoside monomers (e.g., a polymer comprising more than 2, 3, 4, 5, 6, 7, 8, 9, 10, or more nucleotide or nucleoside monomers) more comprising of naturally occurring bases, sugars and internucleotide (backbone) linkages. The term “oligonucleotide” also includes polymers comprising non-naturally occurring monomers, or portions thereof, which function similarly. Such modified oligonucleotides may impart certain benefits often preferred over non-modified forms of the same oligonucleotide sequences, including enhanced cellular uptake and increased stability in the presence of nucleases.

As used herein, the definition of each expression, e.g., alkyl, m, n, etc., when it occurs more than once in any structure, is intended to be independent of its definition elsewhere in the same structure.

As described herein, compounds of the present disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

### Bifunctional Oligonucleotides

The present disclosure features bifunctional oligonucleotides comprising at least two domains, wherein each domain capable of binding to a different target. In an embodiment, the bifunctional oligonucleotide comprises: (i) an alternative splice site targeting sequence and (ii) a spliceosome targeting sequence. In an embodiment, the bifunctional oligonucleotide comprises (i). In an embodiment, the bifunctional oligonucleotide comprises (ii). In an embodiment, the bifunctional oligonucleotide comprises (i) and (ii). In an embodiment, the bifunctional oligonucleotide comprises: (i) a nucleotide sequence capable of binding to a target sequence comprising an alternative splice site (e.g., 5′ splice site), e.g., comprising between 5 and 35 nucleotides in length; and (ii) a nucleotide sequence capable of recruiting a spliceosome component, e.g., an U1 snRNP (e.g., U1 snRNA).

The bifunctional oligonucleotide may be single-stranded or double-stranded. In an embodiment, the bifunctional oligonucleotide is single-stranded. The bifunctional oligonucleotide may be a sense oligonucleotide or an antisense oligonucleotide. In an embodiment, the bifunctional oligonucleotide comprises a sense oligonucleotide. In an embodiment, the bifunctional oligonucleotide comprises an antisense oligonucleotide. In an embodiment, bifunctional oligonucleotide comprises a single-stranded sense oligonucleotide. In an embodiment, bifunctional oligonucleotide comprises a single-stranded antisense oligonucleotide.

The bifunctional oligonucleotide may be anywhere from 10 to 100 nucleotides in length. For example, the bifunctional oligonucleotide may be between 25 and 75 nucleotides in length (e.g, between 25 and 70 nucleotides, between 30 and 65 nucleotides, between 40 and 60 nucleotides). In an embodiment, the bifunctional oligonucleotide comprises an alternative splice site targeting sequence greater than 5, 10, 15, 20, 25, 30, 35, or 40 nucleotides in length. In an embodiment, the bifunctional oligonucleotide comprises an alternative splice site targeting sequence between 5 and 35 nucleotides in length (e.g., between 6 and 35, 7 and 35, 8 and 35, 9 and 35, 10 and 35, 11 and 35, 12 and 35, 13 and 35, 14 and 35, 15 and 35, 16 and 35, 17 and 35, 18 and 35, 19 and 35, 20 and 35,21 and 35,22 and 35,23 and 35,24 and 35, or 25 and 35 nucleotides in length). In an embodiment, the bifunctional oligonucleotide comprises a spliceosome targeting sequence greater than 5, 10, 15, 20, 25, 30, 35, or 40 nucleotides in length.

In an embodiment, the bifunctional oligonucleotide comprises a spliceosome targeting sequence between 5 and 35 nucleotides in length (e.g., between 6 and 35, 7 and 35, 8 and 35, 9 and 35, 10 and 35, 11 and 35, 12 and 35, 13 and 35, 14 and 35, 15 and 35, 16 and 35, 17 and 35, 18 and 35, 19 and 35, 20 and 35, 21 and 35, 22 and 35, 23 and 35, 24 and 35, or 25 and 35 nucleotides in length). In an embodiment, the bifunctional comprises an alternative splice site targeting sequence between 5 and 35 nucleotides in length and a spliceosome targeting sequence between 5 and 35 nucleotides in length.

In an embodiment, the alternative splice site targeting sequence within the bifunctional oligonucleotide is 5′ to the spliceosome targeting sequence. In an embodiment, the alternative splice site targeting sequence within the bifunctional oligonucleotide is 3′ to the spliceosome targeting sequence. In an embodiment, the bifunctional oligonucleotide comprises a plurality of alternative splice site targeting sequences. In an embodiment, the bifunctional oligonucleotide comprises a plurality of spliceosome targeting sequences.

In an embodiment, the bifunctional oligonucleotide comprises an oligonucleotide of Formula (I):

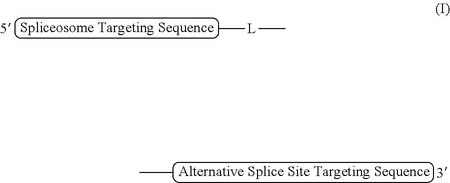

or a pharmaceutically acceptable salt thereof, wherein the alternative splice site targeting sequence is capable of binding to a target sequence (e.g., an RNA, e.g., a pre-mRNA or mRNA) comprising an exonic element, such as an alternative splice site; the spliceosome targeting sequence is capable of binding to a spliceosome component (e.g., U1 snRNP), and L is absent or a linker.

In an embodiment, the bifunctional nucleotide of Formula (I) is a bifunctional nucleotide of Formula (I-a):

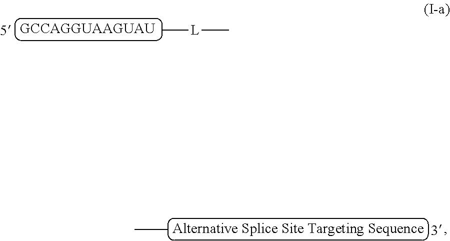

or a pharmaceutically acceptable salt thereof, wherein the alternative splice site targeting sequence is a sequence selected from AAAAGCAGAACCUGAGCGGC, UUCCAGGGUCGCCATGGCGG, UCAGCUTUUCCAGGGUCGCC, AAGGACTUGAGGGACUCGAA, and AAGGACTUGAGGGACUCGAA; each of bases 1-33 may be optionally modified with one or modifications selected from a 2′OMe modification, a locked nucleic acid modification, a 2′-O-methoxy ethyl modification, and phosphorothioate modification; and L is absent or a linker.

In an embodiment, the bifunctional nucleotide of Formula (I) is a bifunctional nucleotide of Formula (I-b):

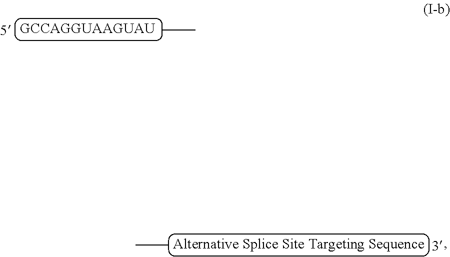

or a pharmaceutically acceptable salt thereof, wherein the alternative splice site targeting sequence is a sequence selected from AAAAGCAGAACCUGAGCGGC, UUCCAGGGUCGCCATGGCGG, UCAGCUTUUCCAGGGUCGCC, AAGGACTUGAGGGACUCGAA, and AAGGACTUGAGGGACUCGAA; and each of bases 1-33 may be optionally modified with one or modifications selected from a 2′OMe modification, a locked nucleic acid modification, a 2′-O-methoxy ethyl modification, and a phosphorothioate modification.

In an embodiment, the bifunctional nucleotide of Formula (I) is a bifunctional nucleotide of Formula (I-c):

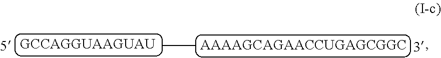

or a pharmaceutically acceptable salt thereof, wherein each of the bases from 1-33 may be optionally modified with one or modifications selected from: 2′OMe modification, locked nucleic acid modification, a 2′-O-methoxy ethyl modification, and phosphorothioate modification.

In an embodiment, the bifunctional nucleotide of Formula (I) is a bifunctional nucleotide of Formula (1-d):

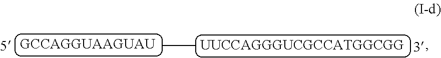

or a pharmaceutically acceptable salt thereof, wherein each of the bases from 1-33 may be optionally modified with one or modifications selected from: 2′OMe modification, locked nucleic acid modification, a 2′-O-methoxy ethyl modification, and phosphorothioate modification.

In an embodiment, the bifunctional nucleotide of Formula (I) is a bifunctional nucleotide of Formula (I-e):

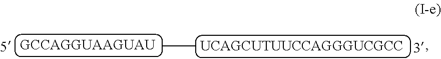

or a pharmaceutically acceptable salt thereof, wherein each of the bases from 1-33 may be optionally modified with one or modifications selected from: 2′OMe modification, locked nucleic acid modification, a 2′-O-methoxy ethyl modification, and phosphorothioate modification.

In an embodiment, the bifunctional nucleotide of Formula (I) is a bifunctional nucleotide of Formula (I-f):

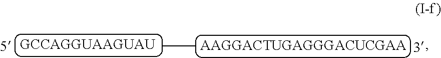

or a pharmaceutically acceptable salt thereof, wherein each of the bases from 1-33 may be optionally modified with one or modifications selected from: 2′OMe modification, locked nucleic acid modification, a 2′-O-methoxy ethyl modification, and phosphorothioate modification.

In an embodiment, the bifunctional nucleotide of Formula (I) is a bifunctional nucleotide of Formula (I-g):

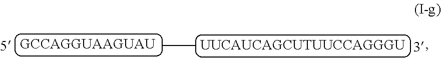

or a pharmaceutically acceptable salt thereof, wherein each of the bases from 1-33 may be optionally modified with one or modifications selected from: 2′OMe modification, locked nucleic acid modification, a 2′-O-methoxy ethyl modification, and phosphorothioate modification.

In an embodiment, the bifunctional oligonucleotide comprises an oligonucleotide of Formula (II):

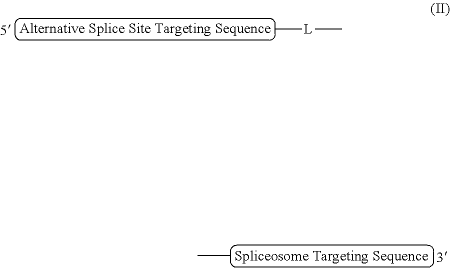

or a pharmaceutically acceptable salt thereof, wherein the alternative splice site targeting sequence is capable of binding to a target sequence (e.g., an RNA, e.g., a pre-mRNA or mRNA) comprising an exonic element, such as an alternative splice site; the spliceosome targeting sequence is capable of binding to a spliceosome component (e.g., U1 snRNP), and L is absent or a linker.

In an embodiment, the bifunctional nucleotide of Formula (II) is a bifunctional nucleotide of Formula (II-a):

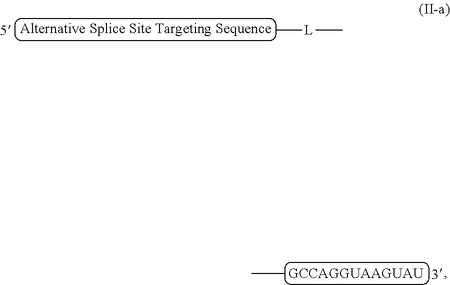

or a pharmaceutically acceptable salt thereof, wherein the alternative splice site targeting sequence is a sequence selected from: AAAAGCAGAACCUGAGCGGC, UUCCAGGGUCGCCATGGCGG, UCAGCUTUUCCAGGGUCGCC, AAGGACTUGAGGGACUCGAA, and AAGGACTUGAGGGACUCGAA; each of bases 1-33 may be optionally modified with one or modifications selected from: 2′OMe modification, locked nucleic acid modification, a 2′-O-methoxy ethyl modification, and phosphorothioate modification; and L is absent or a linker.

A bifunctional oligonucleotide described herein may comprise a chemical modification, such as a non-naturally occurring chemical modification or a naturally occurring chemical modification). The chemical modification may be present at any location on the bifunctional oligonucleotide, including on a nucleotide or at one or both termini of the bifunctional oligonucleotide. In an embodiment, the chemical modification comprises a sugar modification, a nucleobase modification, a terminal modification, or an internucleotide linkage modification. In an embodiment, the chemical modification comprises a sugar modification (e.g, a 2′-ribose modification). Exemplary sugar modifications include a 2′-O-alkyl modification, a 2′-halo modification, or a 2′-deoxy modification (e.g., a 2′-OMe, 2′-OEt, 2′-MOE, 2′-H, 2′-Cl, or 2′-F modification). In an embodiment, the sugar modification comprises a 2′-OMe modification. In an embodiment, the sugar modification comprises a 2′-OMe modification. In an embodiment, the sugar modification comprises a 2′-OEt modification. In an embodiment, the sugar modification comprises a 2′-MOE modification. In an embodiment, the sugar modification comprises a 2′-H modification. In an embodiment, the sugar modification comprises a 2′-Cl modification. In an embodiment, the sugar modification comprises a 2′-F modification. In an embodiment, the chemical modification is a locked nucleic acid (LNA). The term “locked nucleic acid” refers to a modification which the 2′-OH on the nucleotide sugar is connected by an alkylene (e.g., methylene) bridge to the 4′ carbon of the same nucleotide sugar.

The bifunctional oligonucleotide can include modification of all or some of the sugar moieties of the nucleic acid. For example, the 2′ hydroxyl group (OH) of one or more sugar moieties within the bifunctional oligonucleotide sequence can be modified or replaced with a number of different “oxy” or “deoxy” substituents. Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; O—R′ (R′═NH₂, alkylamino, dialkylamino, heterocyclyl, arylamino, diaryl amino, heteroaryl amino, diheteroaryl amino, ethylene diamine, polyamino or aminoalkoxy) and O(CH₂)ₙR′, (e.g., R′═NH₂, alkylamino, dialkylamino, heterocyclyl, arylamino, diaryl amino, heteroaryl amino, or diheteroaryl amino, ethylene diamine, polyamino or aminoalkoxy).

Additional sugar modifications include amino (e.g. NH2; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂, alkylamino, dialkylamino, heterocyclyl, arylamino, diaryl amino, heteroaryl amino, or diheteroaryl amino), NHC(O)R (R=alkyl, cycloalkyl, aryl, aralkyl, heteroaryl or sugar); cyano; mercapto; alkyl-thio-alkyl; thioalkoxy; alkyl; cycloalkyl; aryl; alkenyl and alkynyl, which may be optionally substituted with e.g., an amino functionality.

The sugar group can also contain one or more carbons that possess the opposite stereochemical configuration than that of the corresponding carbon in ribose. Thus, an oligonucleotide can include nucleotides containing e.g., arabinose, as the sugar. The monomer can have an alpha linkage at the 1′ position on the sugar, e.g., alpha-nucleosides. Bifunctional oligonucleotides described herein can also include “abasic” sugars, which lack a nucleobase at C-1′. These abasic sugars can also be further containing modifications at one or more of the constituent sugar atoms. Bifunctional oligonucleotides described herein can also contain one or more sugars that are in the L form, e.g. L-nucleosides. One or more nucleotides of a bifunctional oligonucleotide may have L-sugar with modifications in place of the modified nucleoside in its entity pursuant to the invention described. The L-sugar may have the same sugar and base modification or combinations thereof as in D-sugar. One or more nucleotides of an bifunctional oligonucleotide having the L-sugar may have a 2′-5′ linkage or inverted linkages, e.g. 3′-3′, 5′-5′, 2′-2′ or 2′-3′ linkages. These linkages can be placed between two L-sugar moieties, between L- and D-sugars or between two D-sugars in an oligonucleotide bearing a modified L-nucleoside Modification to the sugar group may also include replacement of the 4′-0 with a sulfur, nitrogen or CH₂ group.

In addition, one or more nucleotides of a bifunctional oligonucleotide may contain an L-sugar with modifications in place of the modified nucleoside. The L-sugar has the same sugar and base modification or combinations thereof as in D-sugar. One or more nucleotides of a bifunctional oligonucleotide having the L-sugar may have a 2′-5′ linkage or inverted linkages, e.g. 3′-3′, 5′-5′, 2′-2′ or 2′-3′ linkages. These linkages can be placed between two L-sugar moieties, between L- and D-sugars or between two D-sugars in an oligonucleotide bearing a modified L-nucleoside. The 3′ and 5′ ends of an oligonucleotide can be modified. Such modifications can be at the 3′ end, 5′ end or both ends of the molecule. They can include modification or replacement of an entire terminal phosphate or of one or more of the atoms of the phosphate group. For example, the 3′ and 5′ ends of a bifunctional oligonucleotide can be conjugated to other functional molecular entities such as labeling moieties, e.g., fluorophores (e.g., pyrene, TAMRA, fluorescein, Cy3 or Cy5 dyes) or protecting groups (for example, comprising a sulfur atom, silicon atom, boron atom, or other moiety, including an acyl or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar.

In an embodiment, the chemical comprises a nucleobase modification (e.g., methylation).

In an embodiment, the chemical modification comprises an internucleotide linkage modification (e.g., a phosphorothioate modification). In an embodiment, a bifunctional oligonucleotide comprises internucletside linkages selected from phosphorus and non-phosphorus containing internucleotide. In one example, the phosphorus containing internucleotide includes, but not limited to, phosphodiester, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity where one or more internucleotide linkages is a 3′ to 3′, 5′ to 5′ or 2′ to 2′ linkage. A bifunctional oligonucleotide described herein may have inverted polarity and can comprise a single 3′ to 3′ linkage at the 3′-most inter-nucleotide linkage i.e. a single inverted nucleoside residue which may be abasic (the nucleobase is missing or has a hydroxyl group in place thereof). Various salts, mixed salts and free acid forms are also included. Representative U.S. patents that describe the preparation of the above phosphorus-containing linkages include U.S. Pat. Nos. 5,194,599; 5,565,555; 5,527,899; 5,721,218; 5,672,697 and 5,625,050, each of which is herein incorporated by reference.

Additional chemical modifications that may be present on a bifunctional oligonucleotide describe herein include 7-deaza-adenosine, N1-methyl-adenosine, N6, N6 (dimethyl)adenine, N6-cis-hydroxy-isopentenyl-adenosine, thio-adenosine, 2-(amino)adenine, 2-(aminopropyl)adenine, 2-(methylthio) N6 (isopentenyl)adenine, 2-(alkyl)adenine, 2-(aminoalkyl)adenine, 2-(aminopropyl)adenine, 2-(halo)adenine, 2-(propyl)adenine, 2′-azido-2′-deoxy-adenosine, 2′-Deoxy-2′-alpha-aminoadenosine, 2′-deoxy-2′-alpha-azidoadenosine, 6-(alkyl)adenine, 6-(methyl)adenine, 6-(alkyl)adenine, 6-(methyl)adenine, 7-(deaza)adenine, 8-(alkenyl)adenine, 8-(alkynyl)adenine, 8-(amino)adenine, 8-(thioalkyl)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)-adenine, 8-(thiol)adenine, 8-azido-adenosine, azaadenine, deazaadenine, N6-(methyl)adenine, N6-(isopentyl)adenine, 7-deaza-8-aza-adenosine, 7-methyladenine, 1-deazaadenosine, 2′-Fluoro-N6-Bz-deoxyadenosine, 2′-OMe-2-amino-adenosine, 2′O-methyl-N6-Bz-deoxyadenosine, 2′-alpha-ethynyladenosine, 2-aminoadenine, 2-Aminoadenosine, 2-Amino-adenosine, 2′-alpha-trifluoromethyladenosine, 2-azidoadenosine, 2′-beta-ethynyladenosine, 2-Bromoadenosine, 2′-beta-Trifluoromethyladenosine, 2-Chloroadenosine, 2′-Deoxy-2′,2′-difluoroadenosine, 2′-Deoxy-2′-alpha-mercaptoadenosine, 2′-Deoxy-2′-alpha-thiomethoxyadenosine, 2′-Deoxy-2′-beta-aminoadenosine, 2′-Deoxy-2′-beta-azidoadenosine, 2′-Deoxy-2′-beta-bromoadenosine, 2′-Deoxy-2′-beta-chloroadenosine, 2′-Deoxy-2′-beta-fluoroadenosine, 2′-Deoxy-2′-beta-iodoadenosine, 2′-Deoxy-2′-beta-mercaptoadenosine, 2′-Deoxy-2′-beta-thiomethoxyadenosine, 2-Fluoroadenosine, 2-Iodoadenosine, 2-Mercaptoadenosine, 2-methoxy-adenine, 2-methylthio-adenine, 2-Trifluoromethyladenosine, 3-Deaza-3-bromoadenosine, 3-Deaza-3-chloroadenosine, 3-Deaza-3-fluoroadenosine, 3-Deaza-3-iodoadenosine, 3-Deazaadenosine, 4′-Azidoadenosine, 4′-Carbocyclic adenosine, 4′-Ethynyladenosine, 5′-Homo-adenosine, 8-Aza-adenosine, 8-bromo-adenosine, 8-Trifluoromethyladenosine, 9-Deazaadenosine, 2-aminopurine, 7-deaza-2,6-diaminopurine, 7-deaza-8-aza-2,6-diaminopurine, 7-deaza-8-aza-2-aminopurine, 2,6-diaminopurine, 7-deaza-8-aza-adenine, 7-deaza-2-aminopurine, 4-methylcytidine, 5-aza-cytidine, Pseudo-iso-cytidine, pyrrolo-cytidine, alpha-thio-cytidine, 2-(thio)cytosine, 2′-Amino-2′-deoxy-cytosine, 2′-Azido-2′-deoxy-cytosine, 2′-Deoxy-2′-alpha-aminocytidine, 2′-Deoxy-2′-alpha-azidocytidine, 3 (deaza) 5 (aza)cytosine, 3 (methyl)cytosine, 3-(alkyl)cytosine, 3-(deaza) 5 (aza)cytosine, 3-(methyl)cytidine, 4,2′-O-dimethylcytidine, 5 (halo)cytosine, 5 (methyl)cytosine, 5 (propynyl)cytosine, 5 (trifluoromethyl)cytosine, 5-(alkyl)cytosine, 5-(alkynyl)cytosine, 5-(halo)cytosine, 5-(propynyl)cytosine, 5-(trifluoromethyl)cytosine, 5-bromo-cytidine, 5-iodo-cytidine, 5-propynyl cytosine, 6-(azo)cytosine, 6-aza-cytidine, aza cytosine, deaza cytosine, N4 (acetyl)cytosine, 1-methyl-1-deaza-pseudoisocytidine, 1-methyl-pseudoisocytidine, 2-methoxy-5-methyl-cytidine, 2-methoxy-cytidine, 2-thio-5-methyl-cytidine, 4-methoxy-1-methyl-pseudoisocytidine, 4-methoxy-pseudoisocytidine, 4-thio-1-methyl-1-deaza-, pseudoisocytidine, 4-thio-1-methyl-pseudoisocytidine, 4-thio-pseudoisocytidine, 5-aza-zebularine, 5-methyl-zebularine, pyrrolo-pseudoisocytidine, zebularine, (E)-5-(2-Bromo-vinyl)cytidine, 2,2′-anhydro-cytidine, 2′-Fluor-N4-Bz-cytidine, 2′-Fluoro-N4-Acetyl-cytidine, 2′-O-Methyl-N4-Acetyl-cytidine, 2′-O-methyl-N4-Bz-cytidine, 2′-a-Ethynylcytidine, 2′-a-Trifluoromethylcytidine, 2′-b-Ethynylcytidine, 2′-b-Trifluoromethylcytidine, 2′-Deoxy-2′,2′-difluorocytidine, 2′-Deoxy-2′-alpha-mercaptocytidine, 2′-Deoxy-2′-alpha-thiomethoxycytidine, 2′-Deoxy-2′-betab-aminocytidine, 2′-Deoxy-2′-beta-azidocytidine, 2′-Deoxy-2′-beta-bromocytidine, 2′-Deoxy-2′-beta-chlorocytidine, 2′-Deoxy-2′-beta-fluorocytidine, 2′-Deoxy-2′-beta-iodocytidine, 2′-Deoxy-2′-beta-mercaptocytidine, 2′-Deoxy-2′-beta-thiomethoxycytidine TP, 2′-O-Methyl-5-(1-propynyl)cytidine, 3′-Ethynylcytidine, 4′-Azidocytidine, 4′-Carbocyclic cytidine, 4′-Ethynylcytidine, 5-(1-Propynyl)ara-cytidine, 5-(2-Chloro-phenyl)-2-thiocytidine, 5-(4-Amino-phenyl)-2-thiocytidine, 5-Aminoallyl-cytosine, 5-Cyanocytidine, 5-Ethynylara-cytidine, 5-Ethynylcytidine, 5′-Homo-cytidine, 5-Methoxycytidine, 5-Trifluoromethyl-Cytidine, N4-Amino-cytidine, N4-Benzoyl-cytidine, pseudoisocytidine, 6-thio-guanosine, 7-deaza-guanosine, 8-oxo-guanosine, Ni-methyl-guanosine, alpha-thio-guanosine, 2-(propyl)guanine, 2-(alky1)guanine, 2′-Amino-2′-deoxy-guanosine, 2′-Azido-2′-deoxy-guanosine, 2′-Deoxy-2′-alpha-aminoguanosine, 2′-Deoxy-2′-alpha-azidoguanosine, 6-(methyl)guanine, 6-(alky1)guanine, 6-(methyl)guanine, 6-methyl-guanosine, 7-(alkyl)guanine, 7-(deaza)guanine, 7-(methyl)guanine, 7-(alkyl)guanine, 7-(deaza)guanine, 7-(methyl)guanine, 8-(alkyl)guanine, 8-(alkynyl)guanine, 8-(halo)guanine, 8-(thioalkyl)guanine, 8-(alkenyl)guanine, 8-(alkyl)guanine, 8-(alkynyl)guanine, 8-(amino)guanine, 8-(halo)guanine, 8-(hydroxyl)guanine, 8-(thioalkyl)guanine, 8-(thiol)guanine, azaguanine, deaza guanine, N(methyl)guanine, N-(methyl)guanine, 1-methyl-6-thio-guanosine, 6-methoxy-guanosine, 6-thio-7-deaza-8-aza-guanosine, 6-thio-7-deaza-guanosine, 6-thio-7-methyl-guanosine, 7-deaza-8-aza-guanosine, 7-methyl-8-oxo-guanosine, N2,N2-dimethyl-6-thio-guanosine, N2-methyl-6-thio-guanosine, 1-Me-guanosine, 2′Fluoro-N2-isobutyl-guanosine, 2′O-methyl-N2-isobutyl-guanosine, 2′-alpha-Ethynylguanosine, 2′-alpha-Trifluoromethyl-guanosine, 2′-beta-Ethynylguanosine, 2′-beta-Trifluoromethylguanosine, 2′-Deoxy-2′,2′-difluoroguanosine, 2′-Deoxy-2′-alpha-mercaptoguanosine, 2′-Deoxy-2′-alpha-thiomethoxyguanosine, 2′-Deoxy-2′-beta-aminoguanosine, 2′-Deoxy-2′-beta-azidoguanosine, 2′-Deoxy-2′-beta-bromoguanosine, 2′-Deoxy-2′-beta-chloroguanosine, 2′-Deoxy-2′-beta-fluoroguanosine, 2′-Deoxy-2′-beta-iodoguanosine, 2′-Deoxy-2′-beta-mercaptoguanosine, 2′-Deoxy-2′-beta-thiomethoxyguanosine, 4′-Azidoguanosine, 4′-Carbocyclic guanosine, 4′-Ethynylguanosine, 5′-Homo-guanosine, 8-bromo-guanosine, 9-Deazaguanosine, N2-isobutyl-guanosine, 7-methylinosine, allyamino-thymidine, aza thymidine, deaza thymidine, deoxy-thymidine, 5-propynyl uracil, alpha-thio-uridine, 1-(aminoalkylamino-carbonylethylenyl)-2(thio)-pseudouracil, 1-(aminoalkylaminocarbonylethylenyl)-2,4-(dithio)pseudouracil, 1-(aminoalkylaminocarbonylethylenyl)-4 (thio)pseudouracil, 1-(aminoalkylamino-carbonylethylenyl)-pseudouracil, 1-(aminocarbonylethylenyl)-2(thio)-, pseudouracil, 1-(aminocarbonylethylenyl)-2,4-, (dithio)pseudouracil, 1-(aminocarbonylethylenyl)-4 (thio)pseudouracil, 1-(aminocarbonylethylenyl)-pseudouracil, 1-substituted 2-(thio)-pseudouracil, 1-substituted 2,4-(dithio)pseudouracil, 1-substituted 4 (thio)pseudouracil, 1-substituted pseudouracil, 1-(aminoalkylamino-carbonylethylenyl)-2-(thio)-pseudouracil, 1-Methyl-3-(3-amino-3-carboxypropyl) pseudouridine, l-Methyl-3-(3-amino-3-carboxyproovl)pseudo-Uradine, 1-Methyl-pseudo-UTP, 2 (thio)pseudouracil, 2′ deoxy uridine, 2′ fluorouridine, 2-(thio)uracil, 2,4-(dithio)psuedouracil, 2′-methyl, 2′-amino, 2′azido, 2′fluro-, guanosine, 2′-Amino-2′-deoxy-uridine, 2′-Azido-2′-deoxy-uridine, 2′-Azido-deoxyuridine, 2′-O-methylpseudouridine, 2′ deoxyuridine, 2′ fluorouridine, 2′-Deoxy-2′-alpha-aminouridine TP, 2′-Deoxy-2′-alpha-azidouridine TP, 2-methylpseudouridine, 3-(3 amino-3-carboxypropyl)uracil, 4-(thio)pseudouracil, 4-(thio)pseudouracil, 4-(thio)uracil, 4-thiouracil, 5-(1,3-diazole-1-alkyl)uracil, 5-(2-aminopropyl)uracil, 5-(aminoalkyl)uracil, 5-(dimethylaminoalkyl)uracil, 5-(guanidiniumalkyl)uracil, 5-(methoxycarbonylmethyl)-2-(thio)uracil, 5-(methoxycarbonyl-methyl)uracil, 5-(methyl)-2-(thio)uracil, 5-(methyl)-2,4-(dithio)uracil, 5 (methyl) 4 (thio)uracil, 5 (methylaminomethyl)-2 (thio)uracil, 5 (methylaminomethyl)-2,4 (dithio)uracil, 5 (methylaminomethyl)-4 (thio)uracil, 5 (propynyl)uracil, 5 (trifluoromethyl)uracil, 5-(2-aminopropyl)uracil, 5-(alky1)-2-(thio)pseudouracil, 5-(alkyl)-2,4 (dithio)pseudouracil, 5-(alky1)-4 (thio)pseudouracil, 5-(alkyl)pseudouracil, 5-(alkyl)uracil, 5-(alkynyl)uracil, 5-(allylamino)uracil, 5-(cyanoalkyl)uracil, 5-(dialkylaminoalkyl)uracil, 5-(dimethylaminoalkyl)uracil, 5-(guanidiniumalkyl)uracil, 5-(halo)uracil, 5-(1,3-diazole-1-alkyl)uracil, 5-(methoxy)uracil, 5-(methoxycarbonylmethyl)-2-(thio)uracil, 5-(methoxycarbonyl-methyl)uracil, 5-(methyl) 2(thio)uracil, 5-(methyl) 2,4 (dithio)uracil, 5-(methyl) 4 (thio)uracil, 5-(methyl)-2-(thio)pseudouracil, 5-(methyl)-2,4 (dithio)pseudouracil, 5-(methyl)-4 (thio)pseudouracil, 5-(methyl)pseudouracil, 5-(methylaminomethyl)-2 (thio)uracil, 5-(methylaminomethyl)-2,4(dithio)uracil, 5-(methylaminomethyl)-4-(thio)uracil, 5-(propynyl)uracil, 5-(trifluoromethyl)uracil, 5-aminoallyl-uridine, 5-bromo-uridine, 5-iodo-uridine, 5-uracil, 6 (azo)uracil, 6-(azo)uracil, 6-aza-uridine, allyamino-uracil, aza uracil, deaza uracil, N3 (methyl)uracil, Pseudo-uridine-1-2-ethanoic acid, pseudouracil, 4-Thio-pseudouridine, 1-carboxymethyl-pseudouridine, 1-methyl-1-deaza-pseudouridine, 1-propynyl-uridine, 1-taurinomethyl-1-methyl-uridine, l-taurinomethyl-4-thio-uridine, 1-taurinomethyl-pseudouridine, 2-methoxy-4-thio-pseudouridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, (±)₁-(2-Hydroxypropyl)pseudouridine, (2R)-1-(2-Hydroxypropyl)pseudouridine, (2S)-1-(2-Hydroxypropyl)pseudouridine, (E)-5-(2-Bromo-vinyl)ara-uridine, (E)-5-(2-Bromo-vinyl)uridine, (Z)-5-(2-Bromo-vinyl)ara-uridine, (Z)-5-(2-Bromo-vinyl)uridine, 1-(2,2,2-Trifluoroethyl)-pseudouridine, 1-(2,2,3,3,3-, Pentafluoropropyl)pseudouridine, 1-(2,2-Diethoxyethy 1)pseudouridine, 1-(2,4,6-Trimethylbenzyl)pseudouridine, 1-(2,4,6-Trimethyl-benzyl)pseudo-uridine, 1-(2,4,6-Trimethyl-phenyl)pseudo-uridine, 1-(2-Amino-2-carboxyethyl)pseudo-uridine, 1-(2-Amino-ethyl)pseudouridine, 1-(2-Hydroxyethyl)pseudouridine, 1-(2-Methoxyethyl)pseudouridine, 1-(3,4-Bis-, trifluoromethoxybenzvl)pseudouridine, 1-(3,4-Dimethoxybenzyl)pseudouridine, 1-(3-Amino-3-carboxypropyl)pseudo-uridine, 1-(3-Amino-propyl)pseudouridine, 1-(3-Cyclopropyl-prop-2-ynyl)pseudouridine TP, 1-(4-Amino-4-carboxybutyl)pseudouridine, 1-(4-Amino-benzyl)pseudouridine, 1-(4-Amino-buty 1)pseudouridine, 1-(4-Amino-phenyl)pseudouridine, 1-(4-Azidobenzyl)pseudouridine, 1-(4-Bromobenzyl)pseudouridine, 1-(4-Chlorobenzyl)pseudouridine, 1-(4-Fluorobenzyl)pseudouridin, 1-(4-Iodobenzyl)pseudouridine, 1-(4-, Methanesulfonvlbenzvl)pseudouridine, 1-(4-Methoxybenzy 1)pseudouridine, 1-(4-Methoxy-benzyl)pseudouridine, 1-(4-Methoxy-phenyl)pseudouridine, 1-(4-Methylbenzyl)pseudouridine, 1-(4-Methyl-benzyl)pseudouridine, 1-(4-Nitrobenzyl)pseudouridine, 1-(4-Nitro-benzy!)pseudouridine, 1(4-Nitro-phenyl)pseudouridine, 1-(4-Thiomethoxybenzyl)pseudouridine, 1-(4-, Trifluoromethoxybenzvl)pseudouridine, 1-(4-Trifluoromethylbenzyl)pseudouridine, 1-(5-Amino-pentyl)pseudouridine, 1-(6-Amino-hexyl)pseudouridine, 1,6-Dimethyl-pseudouridine, 1-[3-(2-{2-[2-(2-Aminoethoxy)-ethoxy]-ethoxy}-ethoxy)-propionyl]pseudouridine, 1-{3-[2-(2-Aminoethoxy)-ethoxy]-propionvl}pseudouridine, 1-Acetylpseudouridine, 1-Alkyl-6-(1-propynyl)-pseudo-uridine, 1-Alkyl-6-(2-propynyl)-pseudo-uridine, 1-Alkyl-6-allyl-pseudo-uridine, 1-Alkyl-6-ethynyl-pseudo-uridine, 1-Alkyl-6-homoallyl-pseudo-uridine, 1-Alkyl-6-vinyl-pseudo-uridine, 1-Allylpseudouridine, 1-Aminomethyl-pseudo-uridine, 1-Benzoylpseudouridine, 1-Benzyloxymethylpseudouridine, 1-Benzyl-pseudo-uridine, 1-Biotinyl-PEG2-pseudouridine, 1-Biotinylpseudouridine, 1-Butyl-pseudo-uridine, 1-Cyanomethylpseudouridine, 1-Cyclobutylmethyl-pseudo-uridine, 1-Cyclobutyl-pseudo-uridine, 1-Cycloheptylmethyl-pseudo-uridine, 1-Cycloheptyl-pseudo-uridine, 1-Cyclohexylmethyl-pseudo-uridine, 1-Cyclohexyl-pseudo-uridine, 1-Cyclooctylmethyl-pseudo-uridine, 1-Cyclooctyl-pseudo-uridine, 1-Cyclopentylmethyl-pseudo-uridine, 1-Cyclopentyl-pseudo-uridine, 1-Cyclopropylmethyl-pseudo-uridine, 1-Cyclopropyl-pseudo-uridine, 1-Ethyl-pseudo-uridine, 1-Hexyl-pseudo-uridine, 1-Homoallylpseudouridine, 1-Hydroxymethylpseudouridine, 1-iso-propyl-pseudo-uridine, 1-Me-2-thio-pseudo-uridine, 1-Me-4-thio-pseudo-uridine, 1-Me-alpha-thio-pseudo-uridine, 1-Methanesulfonylmethylpseudouridine, 1-Methoxymethylpseudouridine uridine, 1-Methyl-6-(2,2,2-Trifluoroethyl)pseudo-uridine, l-Methyl-6-(4-morpholino)-pseudo-uridine, 1-Methyl-6-(4-thiomorpholino)-pseudo-uridine, l-Methyl-6-(substituted phenyl)pseudo-uridine, 1-Methyl-6-amino-pseudo-uridine, 1-Methyl-6-azido-pseudo-uridine, 1-Methyl-6-bromo-pseudo-uridine, 1-Methyl-6-butyl-pseudo-uridine, 1-Methyl-6-chloro-pseudo-uridine, 1-Methyl-6-cyano-pseudo-uridine, 1-Methyl-6-dimethylamino-pseudo-uridine, 1-Methyl-6-ethoxy-pseudo-uridine, 1-Methyl-6-ethylcarboxylate-pseudo-uridine, 1-Methyl-6-ethyl-pseudo-uridine, 1-Methyl-6-fluoro-pseudo-uridine, 1-Methyl-6-formyl-pseudo-uridine, 1-Methyl-6-hydroxyamino-pseudo-uridine, 1-Methyl-6-hydroxy-pseudo-uridine, 1-Methyl-6-iodo-pseudo-uridine, 1-Methyl-6-iso-propyl-pseudo-uridine, 1-Methyl-6-methoxy-pseudo-uridine, 1-Methyl-6-methylamino-pseudo-uridine, 1-Methyl-6-phenyl-pseudo-uridine, 1-Methyl-6-propyl-pseudo-uridine, 1-Methyl-6-tert-butyl-pseudo-uridine, 1-Methyl-6-trifluoromethoxy-pseudo-uridine, 1-Methyl-6-trifluoromethyl-pseudo-uridine, 1-Morpholinomethylpseudouridine, 1-Pentyl-pseudo-uridineuridine, 1-Phenyl-pseudo-uridine, 1-Pivaloylpseudouridine, 1-Propargylpseudouridine, 1-Propyl-pseudo-uridine, 1-propynyl-pseudouridine, 1-p-tolyl-pseudo-uridine, 1-tert-Butyl-pseudo-uridine, 1-Thiomethoxymethylpseudouridine, 1-Thiomorpholinomethylpseudouridine, 1-Trifluoroacetylpseudouridine, 1-Trifluoromethyl-pseudouridine, 1-Vinylpseudouridine, 2,2′-anhydro-uridine, 2′-bromo-deoxyuridine, 2′-F-5-Methyl-2′-deoxy-uridine, 2′-OMe-5-Me-uridine, 2′-OMe-pseudouridine, 2′-alpha-Ethynyluridine, 2′-alpha-Trifluoromethyluridine, 2′-beta-Ethynyluridine, 2′-beta-Trifluoromethyluridiner, 2′-Deoxy-2′,2′-difluorouridine, 2′-Deoxy-2′-a-mercaptouridin, 2′-Deoxy-2′-alpha-thiomethoxyuridine, 2′-Deoxy-2′-beta-aminouridine, 2′-Deoxy-2′-beta-azidouridine, 2′-Deoxy-2′-beta-bromouridine, 2′-Deoxy-2′-beta-chlorouridine, 2′-Deoxy-2′-beta-fluorouridine, 2′-Deoxy-2′-beta-iodouridine, 2′-Deoxy-2′-beta-mercaptouridine, 2′-Deoxy-2′-beta-thiomethoxyuridine, 2-methoxy-4-thio-uridine, 2-methoxyuridine, 2′-O-Methyl-5-(1-propynyl)uridine, 3-Alkyl-pseudo-uridine, 4′-Azidouridine, 4′-Carbocyclic uridine, 4′-Ethynyluridine, 5-(1-Propynyl)ara-uridine, 5-(2-Furanyl)uridine, 5-Cyanouridine, 5-Dimethylaminouridine, 5′-Homo-uridine, 5-iodo-2′-fluoro-deoxyuridine, 5-Phenylethynyluridine, 5-Trideuteromethyl-6-deuterouridine, 5-Trifluoromethyl-Uri dine, 5-Vinylarauridine, 6-(2,2,2-Trifluoroethyl)-pseudo-uridine, 6-(4-Morpholino)-pseudo-uridine, 6-(4-Thiomorpholino)-pseudo-uridine, 6-(Substituted-Phenyl)-pseudo-uridine, 6-Amino-pseudo-uridine, 6-Azido-pseudo-uridine, 6-Bromo-pseudo-uridine, 6-Butyl-pseudo-uridine, 6-Chloro-pseudo-uridine, 6-Cyano-pseudo-uridine, 6-Dimethylamino-pseudo-uridine, 6-Ethoxy-pseudo-uridine, 6-Ethylcarboxylate-pseudo-uridine, 6-Ethyl-pseudo-uridine, 6-Fluoro-pseudo-uridine, 6-Formyl-pseudo-uridine, 6-Hydroxyamino-pseudo-uridine, 6-Hydroxy-pseudo-uridine, 6-Iodo-pseudo-uridine, 6-iso-Propyl-pseudo-uridine, 6-Methoxy-pseudo-uridine, 6-Methylamino-pseudo-uridine, 6-Methyl-pseudo-uridine, 6-Phenyl-pseudo-uridine, 6-Phenyl-pseudo-uridine, 6-Propyl-pseudo-uridine, 6-tert-Butyl-pseudo-uridine, 6-Trifluoromethoxy-pseudo-uridine, 6-Trifluoromethyl-pseudo-uridine, alpha-thio-pseudo-uridine, Pseudouridine 1-(4-methylbenzenesulfonic, acid), Pseudouridine 1-(4-methylbenzoic acid) TP, Pseudouridine 1-[3-(2-, ethoxy)]propionic acid, Pseudouridine 1-[3-{2-(2-[2-(2-ethoxy,)-ethoxy]-ethoxy)-ethoxy}]propionic acid, Pseudouridine 1-[3-{2-(2-[2-{2(2-, ethoxy)-ethoxy}-ethoxy]-ethoxy)-ethoxy}]propionic acid, Pseudouridine 1-[3-{2-(2-[2-ethoxy]-ethoxy)-ethoxy}]propionic acid, Pseudouridine 1-[3-{2-(2-ethoxy)-, ethoxy}]propionic acid, Pseudouridine 1-methylphosphonic, acid, Pseudouridine TP 1-methylphosphonic acid diethyl ester, Pseudo-uridine-N1-3-propionic acid, Pseudo-uridine-N1-4-butanoic acid, Pseudo-uridine-N, 1-5-pentanoic acid, Pseudo-uridine-N1-6-hexanoic acid, Pseudo-uridine-N1-7-heptanoic acid, Pseudo-uridine-N1-methyl-p-benzoic acid, and pseudo-uridine-N1-p-benzoic acid.

In an embodiment, the bifunctional oligonucleotide described herein comprises a plurality of chemical modifications. In an embodiment, the bifunctional oligonucleotide comprises a chemical modification within the alternative splice site targeting sequence. In an embodiment, the bifunctional oligonucleotide comprises a chemical modification within the spliceosome targeting sequence. In an embodiment, the bifunctional oligonucleotide comprises a plurality of chemical modifications within splice site target sequence and the spliceosome targeting sequence. In an embodiment, the bifunctional oligonucleotide comprises a plurality of sugar modifications or LNAs. In an embodiment, the bifunctional oligonucleotide comprises at least 1, 2, 3, 4, 5, 6, 7, 8, 9, 10, 15, 20, 25, 30, or more sugar modifications (e.g., 2′O-Me modifications). In an embodiment, the bifunctional oligonucleotide comprises at least 1, 2, 3, 4, 5, 6, 7, 8, 9, 10, 15, 20, 25, 30, or more LNAs.

The bifunctional oligonucleotide may be targeted to an alternate splice site within a target gene, e.g., a target gene described herein, e.g., a target gene implicated in a repeat expansion disease. In an embodiment, the target gene is the Huntingtin gene (HTT). The nucleic acid sequence of an exemplary Homo sapiens (human) HTT gene is set forth in NCBI Reference NG_009378.1. In an embodiment, the target splice site sequence (e.g., 5′ splice site) is present within the HTT gene. In an embodiment, the target splice sequence is present within exon 1 of the HTT gene. In an embodiment, the target splice site sequence is within exon 1 of the HTT gene. In an embodiment, the target splice site sequence is upstream of a CAG region within exon 1. In an embodiment, the target splice site sequence comprises the sequence GAGT or AAGT. In an embodiment, the target splice site sequence comprises the sequence GAGT. In an embodiment, the target splice site sequence comprises the sequence AAGT.

In an embodiment, the bifunctional oligonucleotide comprises an alternative splice site targeting sequence that binds to a region with the HTT exon 1 sequence (SEQ ID NO: 001). Exemplary alternative splice sites include the underlined sequences GAGT (SEQ ID NO: 002) and AAGT (SEQ ID NO: 003).

HTT Exon 1 Sequence
(SEQ ID NO: 001)
CGAGTCGGCCCGAGGCCTCCGGGGACTGCCGTGCCGGGCGGGAGACCGC
CATGGCGACCCTGGAAAAGCTGATGAAGGCCTTCGAGTCCCTCAAGTCC
TTCCAGCAGCAGCAGCAGCAGCAGCAGCAGCAGCAGCAGCAGCAGCAGC
AGCAGCAGCAGCAACAGCCGCCACCGCCGCCGCCGCCGCCGCCGCCTCC
TCAGCTTCCTCAGCCGCCGCCGCAGGCACAGCCGCTGCTGCCTCAGCCG
CAGCCGCCCCCGCCGCCGCCCCCGCCGCCACCCGGCCCGGCTGTGGCTG
AGGAGCCGCTGCACCGACC

In an embodiment, the spliceosome targeting sequence recognizes the U1 snRNP. In an embodiment, the U1 snRNP is a wild type U1 snRNP or a variant or fragment thereof.

In an embodiment, the bifunctional oligonucleotide has a sequence selected from a sequence provided in Table 1 or 2. In Tables 1 and 2, an “m” placed before a nucleotide refers to a 2′OMe modification, and a “+” placed before a nucleotide refers to a locked nucleic acid (LNA). In an embodiment, the bifunctional oligonucleotide comprises a sequence with at least 75%, 80%, 85%, 90%, 95%, 99%, or more sequence identity with a nucleotide selected from SEQ ID NOs: 100-254, or a variant or fragment thereof. In an embodiment, the bifunctional oligonucleotide comprises the sequence GCCAGGUAAGUAU (SEQ ID NO: 004). In an embodiment, the bifunctional oligonucleotide comprises a sequence with at least 75%, 80%, 85%, 90%, 95%, 99%, or more sequence identity with SEQ TD NO: 004, or a variant or fragment thereof.

TABLE 1
Exemplary bifunctional oligonucleotides

| SEQ ID | | Molecular |
|---|---|---|
| NO. | Sequence | Weight |
| 100 | mGmCmCmAmGmG mUmAmAmGmUmAmUmAmU + Cm | 11053.3 |
| | UmUmG + GmAmCmC + CmGmUmC + CmCmGmG + CmA | |
| 101 | mGmCmCmAmGmGmUmAmA mGmUmAmUmCmA + | 11015.2 |
| | TmCmUmU + GmGmAmC + C mCmGmU + CmCmCmG + GmC | |
| 102 | mGmCmCmAmGmG mUmAmA mGmUmAmUmCmC + | 11015.2 |
| | AmUmCmU + TmG mGmA + C mCmCmG + TmCmC mC + GmG | |
| 103 | mGmCmCmAmGmG mUmAmA mGmUmAmUmUmC + | 10976.2 |
| | CmAmUmC + TmUmGmG + A mCmCmC + GmUmCmC + CmG | |
| 104 | mGmCmCmAmGmGmUmAmAmGmUmAmUmGmU + | 10990.2 |
| | CmCmAmU + CmUmUmG + G mAmCmC + CmGmUmC + CmC | |
| 105 | mGmCmCmAmGmG mUmAmA mGmUmAmUmCmG + | 10990.2 |
| | TmCmC mA + TmC mUmU + GmGmAmC + CmCmGmU + CmC | |
| 106 | mGmCmCmAmGmG mUmAmA mGmUmA mUmCmC + | 10976.2 |
| | GmUmCmC + AmUmCmU + T mGmGmA + CmCmCmG + TmC | |
| 107 | mGmCmC mAmGmG mUmAmA mGmUmAmUmGmC + | 11016.2 |
| | CmGmU mC + CmA mUmC + TmUmGmG + AmCmC mC + GmU | |
| 108 | mGmCmCmAmGmG mUmAmAmGmUmA mUmGmG + | 11069.3 |
| | CmCmGmU + CmC mAmU + CmUmUmG + GmAmCmC + CmG | |
| 109 | mGmCmCmAmGmGmUmAmA mGmUmAmUmCmG + | 11015.2 |
| | GmCmCmG + TmCmCmA + T mCmUmU + GmGmA mC + CmC | |
| 110 | mGmCmCmAmGmGmUmAmAmGmUmAmUmGmC + | 11041.2 |
| | GmGmCmC + GmUmCmC + A mUmCmU + TmGmG mA + CmC | |
| 111 | mGmCmCmAmGmGmUmAmAmGmUmAmUmAmG + | 11093.3 |
| | CmGmG mC + CmGmUmC + CmAmUmC + TmUmG mG + AmC | |
| 112 | mGmCmCmAmGmGmUmAmAmGmUmAmUmGmA + | 11119.3 |
| | GmCmG mG + CmCmGmU + CmCmAmU + CmUmU mG + GmA | |
| 113 | mGmCmCmAmGmG mUmAmAmGmUmAmUmUmG + | 11082.2 |
| | AmGmC mG + GmC mCmG + TmCmCmA + TmCmU mU + GmG | |
| 114 | mGmCmCmAmGmG mUmAmA mGmUmAmUmCmU + | 11028.2 |
| | GmAmGmC + GmGmCmC + G mUmCmC + AmUmC mU + TmG | |
| 115 | mGmCmCmAmGmGmUmAmAmGmUmAmUmCmC + | 11044.3 |
| | TmGmAmG + CmGmGmC + CmGmUmC + CmAmUmC + TmU | |
| 116 | mGmCmCmAmGmGmUmAmA mGmUmAmUmAmC + | 11053.3 |
| | CmUmG mA + GmCmGmG + C mCmGmU + CmCmA | |
| | mU + CmU | |
| 117 | mGmCmCmAmGmGmUmAmAmGmUmAmUmAmA + | 11062.3 |
| | CmCmUmG + AmGmCmG + G mCmCmG + TmCmC mA + TmC | |
| 118 | mGmCmCmAmGmGmUmAmAmGmUmAmUmGmA + | 11060.2 |
| | AmCmCmU + GmA mGmC + GmGmCmC + GmUmC mC + AmU | |
| 119 | mGmCmCmAmGmGmUmAmAmGmUmAmUmAmG + | 11139.4 |
| | AmAmCmC + TmG mAmG + C mGmGmC + CmGmUmC + CmA | |
| 120 | mGmCmCmAmGmG mUmAmAmGmUmAmUmCmA + | 11101.3 |
| | GmAmAmC + CmU mGmA + GmCmGmG + CmCmG mU + CmC | |
| 121 | mGmCmCmAmGmGmUmAmAmGmUmAmUmGmC + | 11127.3 |
| | AmGmAmA + CmCmUmG + A mGmCmG + GmCmC mG + TmC | |
| 122 | mGmCmCmAmGmG mUmAmAmGmUmAmUmAmG + | 11137.3 |
| | CmAmGmA + AmC mCmU + GmAmGmC + GmGmC mC + GmU | |
| 123 | mGmCmCmAmGmG mUmAmA mGmUmAmUmAmA + | 11188.4 |
| | GmCmAmG + AmA mCmC + T mGmAmG + CmGmG | |
| | mC + CmG | |
| 124 | mGmCmCmAmGmGmUmAmAmGmUmAmUmAmA + | 11158.4 |
| | AmGmCmA + GmAmAmC + C mUmGmA + GmCmG mG + CmC | |
| 125 | mGmCmCmAmGmG mUmAmAmGmUmAmUmAmA + | 11168.4 |
| | AmAmGmC + AmG mAmA + CmCmUmG + AmGmC mG + GmC | |
| 126 | mGmCmCmAmGmGmUmAmAmGmUmAmUmUmA + | 11169.4 |
| | AmAmAmG + CmAmGmA + AmCmCmU + GmAmG mC + GmG | |
| 127 | mGmCmCmAmGmG mUmAmAmGmUmAmUmGmU + | 11183.4 |
| | AmAmAmA + GmC mAmG + A mAmCmC + TmGmA | |
| | mG + CmG | |
| 128 | mGmCmCmAmGmGmUmAmAmGmUmAmUmGmG + | 11183.4 |
| | TmAmAmA + AmGmCmA + G mAmAmC + CmUmG mA + GmC | |
| 129 | mGmCmCmAmGmGmUmAmAmGmUmAmUmAmG + | 11193.4 |
| | GmUmAmA + AmAmGmC + AmGmAmA + CmCmUmG + AmG | |
| 130 | mGmCmCmAmGmGmUmAmAmGmUmAmUmCmA + | 11153.4 |
| | GmGmUmA + AmA mAmG + C mAmGmA + AmCmC | |
| | mU + GmA | |
| 131 | mGmCmCmAmGmGmUmAmAmGmUmAmUmGmC + | 11169.4 |
| | AmGmGmU + AmA mAmA + GmCmAmG + AmAmC mC + TmG | |
| 132 | mGmCmCmAmGmGmUmAmAmGmUmAmUmCmG + | 11157.4 |
| | CmAmGmG + TmAmAmA + A mGmCmA + GmAmA mC + CmU | |
| 133 | mGmCmCmAmGmGmUmAmAmGmUmAmUmCmC + | 11128.4 |
| | GmCmAmG + GmUmAmA + AmAmGmC + AmGmA mA + CmC | |
| 134 | mGmCmCmAmGmGmUmAmAmGmUmAmUmGmC + | 11182.4 |
| | CmGmCmA + GmGmUmA + AmAmAmG + CmAmG mA + AmC | |
| 135 | mGmCmCmAmGmGmUmAmAmGmUmAmUmGmG + | 11208.4 |
| | CmCmGmC + AmGmGmU + AmAmAmA + GmCmA mG + AmA | |
| 136 | mGmCmC mAmGmG mUmAmA mGmUmAmUmGmG + | 11238.4 |
| | GmCmCmG + CmAmGmG + T mAmAmA + AmGmC mA + GmA | |
| 137 | mGmCmCmAmGmGmUmAmAmGmUmAmUmUmG + | 11187.4 |
| | GmGmCmC + GmCmAmG + G mUmAmA + AmAmG | |
| | mC + AmG | |
| 138 | mGmCmCmAmGmGmUmAmAmGmUmAmUmCmU + | 11175.4 |
| | GmGmGmC + CmGmCmA + GmGmUmA + AmAmA mG + CmA | |
| 139 | mGmCmCmAmGmGmUmAmAmGmUmAmUmUmC + | 11152.3 |
| | TmGmGmG + CmCmGmC + AmGmGmU + AmAmA mA + GmC | |
| 140 | mGmCmC mAmGmG mUmAmA mGmUmA mUmCmU + | 11166.4 |
| | CmUmG mG + GmC mCmG + C mAmGmG + TmAmA | |
| | mA + AmG | |
| 141 | mGmCmCmAmGmGmUmAmAmGmUmAmUmGmC + | 11138.3 |
| | TmCmUmG + GmGmCmC + GmCmAmG + GmUmA mA + AmA | |
| 142 | mGmCmCmAmGmGmUmAmAmGmUmAmUmGmG + | 11168.3 |
| | CmUmCmU + GmGmGmC + CmGmCmA + GmGmU mA + AmA | |
| 143 | mGmCmCmAmGmGmUmAmAmGmUmAmUmGmG + | 11184.3 |
| | GmCmUmC + TmG mGmG + C mCmGmC + AmGmG | |
| | mU + AmA | |
| 144 | mGmCmCmAmGmGmUmAmAmGmUmAmUmUmC + | 11039.3 |
| | GmCmGmG + CmGmCmC + G mCmUmC + AmGmC mA + CmC | |
| 145 | mGmCmC mAmGmG mUmAmA mGmUmA mUmCmU + | 11053.3 |
| | CmGmC mG + GmC mGmC + C mGmCmU + CmAmG | |
| | mC + AmC | |
| 146 | mGmCmCmAmGmGmUmAmAmGmUmAmUmAmC + | 11091.3 |
| | TmCmG mC + GmGmCmG + CmCmGmC + TmCmA mG + CmA | |
| 147 | mGmCmCmAmGmG mUmAmAmGmUmAmUmGmA + | 11093.3 |
| | CmUmCmG + CmG mGmC + G mCmCmG + CmUmC mA + GmC | |
| 148 | mGmCmCmAmGmGmUmAmAmGmUmAmUmCmG + | 11065.3 |
| | AmCmUmC + GmC mGmG + CmGmCmC + GmCmU mC + AmG | |
| 149 | mGmCmCmAmGmGmUmAmAmGmUmAmUmCmC + | 11053.3 |
| | GmAmC mU + CmG mCmG + G mCmGmC + CmGmC | |
| | mU + CmA | |
| 150 | mGmCmCmAmGmGmUmAmAmGmUmAmUmAmG + | 11147.4 |
| | GmCmCmU + CmGmGmG + C mCmGmA + CmUmC mG + CmG | |
| 151 | mGmCmCmAmGmGmUmAmAmGmUmAmUmGmA + | 11105.3 |
| | GmGmCmC + TmCmGmG + G mCmCmG + AmCmU mC + GmC | |
| 152 | mGmCmCmAmGmGmUmAmAmGmUmAmUmGmG + | 11159.3 |
| | AmGmGmC + CmUmCmG + G mGmCmC + GmAmC mU + CmG | |
| 153 | mGmCmCmAmGmGmUmAmAmGmUmAmUmCmG + | 11133.3 |
| | GmAmG mG + CmC mUmC + G mGmGmC + CmGmA | |
| | mC + TmC | |
| 154 | mGmCmCmAmGmGmUmAmAmGmUmAmUmCmC + | 11133.3 |
| | GmGmAmG + GmCmCmU + CmGmGmG + CmCmG mA + CmU | |
| 155 | mGmCmCmAmGmGmUmAmAmGmUmAmUmAmG + | 11133.3 |
| | TmCmCmC + CmGmGmA + G mGmCmC + TmCmG mG + GmC | |
| 156 | mGmCmCmAmGmGmUmAmAmGmUmAmUmCmA + | 11119.3 |
| | GmUmCmC + CmCmGmG + AmGmGmC + CmUmC mG + GmG | |
| 157 | mGmCmCmAmGmGmUmAmAmGmUmAmUmGmC + | 11119.3 |
| | AmGmUmC + CmCmCmG + GmAmGmG + CmCmU mC + GmG | |
| 158 | mGmCmCmAmGmGmUmAmAmGmUmAmUmGmG + | 11133.3 |
| | CmAmG mU + CmC mCmC + G mGmAmG + GmCmC | |
| | mU + CmG | |
| 159 | mGmCmCmAmGmGmUmAmAmGmUmAmUmCmG + | 11093.3 |
| | GmCmAmG + TmCmCmC + C mGmGmA + GmGmC mC + TmC | |
| 160 | mGmCmCmAmGmGmUmAmAmGmUmAmUmAmC + | 11103.3 |
| | GmGmCmA + GmU mCmC + CmCmGmG + AmGmG mC + CmU | |
| 161 | mGmCmCmAmGmGmUmAmAmGmUmAmUmCmA + | 11116.3 |
| | CmGmGmC + AmGmUmC + CmCmCmG + GmAmGmG + CmC | |
| 162 | mGmCmCmAmGmGmUmAmAmGmUmAmUmGmC + | 11142.3 |
| | AmCmGmG + CmAmGmU + C mCmCmC + GmGmA | |
| | mG + GmC | |
| 163 | mGmCmCmAmGmGmUmAmAmGmUmAmUmGmG + | 11196.4 |
| | CmAmCmG + GmCmAmG + TmCmCmC + CmGmGmA + GmG | |
| 164 | mGmCmCmAmGmGmUmAmAmGmUmAmUmCmG + | 11128.3 |
| | GmCmAmC + GmGmCmA + G mUmCmC + CmCmG mG + AmG | |
| 165 | mGmCmCmAmGmGmUmAmAmGmUmAmUmCmC + | 11102.3 |
| | GmGmCmA + CmGmGmC + AmGmUmC + CmCmCmG + GmA | |
| 166 | mGmCmCmAmGmGmUmAmAmGmUmAmUmUmC + | 11067.3 |
| | CmCmGmC + CmC mGmG + CmAmCmG + GmCmA mG + TmC | |
| 167 | mGmCmC mAmGmG mUmAmA mGmUmA mUmCmU + | 11039.3 |
| | CmCmC mG + CmC mCmG + G mCmAmC + GmGmC | |
| | mA + GmU | |
| 168 | mGmCmCmAmGmGmUmAmAmGmUmAmUmUmC + | 11039.3 |
| | TmCmCmC + GmCmCmC + G mGmCmA + CmGmGmC + AmG | |
| 169 | mGmCmCmAmGmGmUmAmAmGmUmAmUmGmU + | 11067.3 |
| | CmUmC mC + CmGmCmC + CmGmGmC + AmCmG mG + CmA | |
| 170 | mGmCmCmAmGmGmUmAmAmGmUmAmUmUmG + | 11084.3 |
| | GmCmG mG + TmCmUmC + CmCmGmC + CmCmGmG + CmA | |
| 171 | mGmCmCmAmGmGmUmAmAmGmUmAmUmAmU + | 11056.2 |
| | GmGmCmG + GmUmCmU + CmCmCmG + CmCmCmG + GmC | |
| 172 | mGmCmCmAmGmGmUmAmAmGmUmAmUmCmA + | 11056.2 |
| | TmGmGmC + GmG mUmC + TmCmCmC + GmCmCmC + GmG | |
| 173 | mGmCmCmAmGmGmUmAmAmGmUmAmUmCmC + | 11044.3 |
| | AmUmGmG + CmGmGmU + C mUmCmC + CmGmCmC + CmG | |
| 174 | mGmCmCmAmGmGmUmAmAmGmUmA mUmGmC + | 11044.3 |
| | CmAmUmG + GmCmGmG + T mCmUmC + CmCmG mC + CmC | |
| 175 | mGmCmC mAmGmGmUmAmAmGmUmAmUmCmG + | 11030.2 |
| | CmCmA mU + GmGmCmG + GmUmCmU + CmCmC mG + CmC | |
| 176 | mGmCmCmAmGmGmUmAmAmGmUmAmUmUmC + | 11017.2 |
| | GmCmCmA + TmGmGmC + G mGmUmC + TmCmCmC + GmC | |
| 177 | mGmCmCmAmGmGmUmAmAmGmUmAmUmGmU + | 11085.3 |
| | CmGmCmC + AmUmGmG + CmGmGmU + CmUmCmC + CmG | |
| 178 | mGmCmCmAmGmGmUmAmAmGmUmAmUmGmG + | 11085.3 |
| | TmCmGmC + CmAmUmG + G mCmGmG + TmCmUmC + CmC | |
| 179 | mGmCmCmAmGmGmUmAmAmGmUmAmUmGmG + | 11097.3 |
| | GmUmCmG + CmC mAmU + G mGmCmG + GmUmC | |
| | mU + CmC | |
| 180 | mGmCmCmAmGmGmUmAmAmGmUmAmUmAmG + | 11121.3 |
| | GmGmUmC + GmCmCmA + T mGmGmC + GmGmU mC + TmC | |
| 181 | mGmCmC mAmGmG mUmAmA mGmUmAmUmCmA + | 11135.3 |
| | GmGmG mU + CmG mCmC + A mUmGmG + CmGmG | |
| | mU + CmU | |
| 182 | mGmCmCmAmGmGmUmAmAmGmUmAmUmCmC + | 11134.3 |
| | AmGmGmG + TmC mGmC + CmAmUmG + GmCmG mG + TmC | |
| 183 | mGmCmCmAmGmGmUmAmAmGmUmAmUmUmC + | 11121.3 |
| | CmAmGmG + GmUmCmG + CmCmAmU + GmGmCmG + GmU | |
| 184 | mGmCmCmAmGmGmUmAmAmGmUmAmUmUmU + | 11121.3 |
| | CmCmA mG + GmG mUmC + GmCmCmA + TmGmG | |
| | mC + GmG | |
| 185 | mGmCmCmAmGmGmUmAmAmGmUmAmUmUmU + | 11096.3 |
| | TmCmCmA + GmGmGmU + CmGmCmC + AmUmG mG + CmG | |
| 186 | mGmCmCmAmGmGmUmAmAmGmUmAmUmUmU + | 11057.2 |
| | TmUmCmC + AmGmGmG + TmCmGmC + CmAmUmG + GmC | |
| 187 | mGmCmCmAmGmG mUmAmAmGmUmA mUmCmU + | 11057.2 |
| | TmUmUmC + CmAmGmG + GmUmCmG + CmCmA mU + GmG | |
| 188 | mGmCmCmAmGmG mUmAmA mGmUmAmUmGmC + | 11057.2 |
| | TmUmUmU + CmC mAmG + G mGmUmC + GmCmC | |
| | mA + TmG | |
| 189 | mGmCmCmAmGmGmUmAmAmGmUmAmUmAmG + | 11041.2 |
| | CmUmU mU + TmCmCmA + GmGmGmU + CmGmC mC + AmU | |
| 190 | mGmCmCmAmGmGmUmAmAmGmUmA mUmCmA + | 11040.2 |
| | GmCmUmU + TmUmCmC + A mGmGmG + TmCmG mC + CmA | |
| 191 | mGmCmCmAmGmGmUmAmAmGmUmAmUmUmC + | 11017.2 |
| | AmGmC mU + TmUmUmC + CmAmGmG + GmUmC mG + CmC | |
| 192 | mGmCmCmAmGmGmUmAmAmGmUmAmUmAmU + | 11041.2 |
| | CmAmGmC + TmUmUmU + CmCmAmG + GmGmU mC + GmC | |
| 193 | mGmCmCmAmGmG mUmAmAmGmUmAmUmCmA + | 11055.2 |
| | TmCmAmG + CmU mUmU + T mCmCmA + GmGmG mU + CmG | |
| 194 | mGmCmCmAmGmG mUmAmA mGmUmA mUmUmC + | 10988.2 |
| | AmUmCmA + GmC mUmU + T mUmCmC + AmGmG | |
| | mG + TmC | |
| 195 | mGmCmCmAmGmGmUmAmAmGmUmAmUmUmU + | 11003.2 |
| | CmAmUmC + AmG mCmU + TmUmUmC + CmAmG | |
| | mG + GmU | |
| 196 | mGmCmCmAmGmGmUmAmA mGmUmAmUmCmU + | 11016.2 |
| | TmCmAmU + CmAmGmC + T mUmUmU + CmCmA mG + GmG | |
| 197 | mGmCmCmAmGmGmUmAmAmGmUmAmUmCmC + | 10976.2 |
| | TmUmC mA + TmC mAmG + C mUmUmU + TmCmC | |
| | mA + GmG | |
| 198 | mGmCmCmAmGmGmUmAmAmGmUmAmUmGmC + | 10948.1 |
| | CmUmUmC + AmUmCmA + G mCmUmU + TmUmC | |
| | mC + AmG | |
| 199 | mGmCmCmAmGmGmUmAmAmGmUmAmUmGmG + | 10976.2 |
| | CmCmUmU + CmAmUmC + A mGmCmU + TmUmU mC + CmA | |
| 200 | mGmCmCmAmGmG mUmAmA mGmUmAmUmAmG + | 10976.2 |
| | GmCmCmU + TmC mAmU + C mAmGmC + TmUmU | |
| | mU + CmC | |
| 201 | mGmCmCmAmGmGmUmAmAmGmUmAmUmAmA + | 11000.2 |
| | GmGmC mC + TmUmCmA + TmCmAmG + CmUmU mU + TmC | |
| 202 | mGmCmCmAmGmGmUmAmAmGmUmAmUmGmA + | 11012.2 |
| | AmGmGmC + CmUmUmC + AmUmCmA + GmCmU mU + TmU | |
| 203 | mGmCmCmAmGmGmUmAmAmGmUmAmUmCmG + | 11025.2 |
| | AmAmGmG + CmCmUmU + C mAmUmC + AmGmC mU + TmU | |
| 204 | mGmCmCmAmGmGmUmAmA mGmUmAmUmUmC + | 11025.2 |
| | GmAmA mG + GmCmCmU + T mCmAmU + CmAmG mC + TmU | |
| 205 | mGmCmCmAmGmGmUmAmAmGmUmAmUmAmC + | 11033.3 |
| | TmCmGmA + AmG mGmC + C mUmUmC + AmUmC | |
| | mA + GmC | |
| 206 | mGmCmCmAmGmGmUmAmAmGmUmAmUmGmA + | 11087.3 |
| | CmUmCmG + AmAmGmG + CmCmUmU + CmAmUmC + AmG | |
| 207 | mGmCmCmAmGmGmUmAmAmGmUmAmUmGmG + | 11073.3 |
| | AmCmUmC + GmAmAmG + G mCmCmU + TmCmA mU + CmA | |
| 208 | mGmCmCmAmGmGmUmAmAmGmUmAmUmGmG + | 11103.3 |
| | GmAmCmU + CmG mAmA + G mGmCmC + TmUmC mA + TmC | |
| 209 | mGmCmCmAmGmGmUmAmAmGmUmAmUmAmG + | 11113.3 |
| | GmGmAmC + TmC mGmA + AmGmGmC + CmUmUmC + AmU | |
| 210 | mGmCmCmAmGmGmUmAmAmGmUmAmUmGmA + | 11166.4 |
| | GmGmG mA + CmUmCmG + A mAmGmG + CmCmU | |
| | mU + CmA | |
| 211 | mGmCmCmAmGmGmUmAmAmGmUmAmUmUmG + | 11115.3 |
| | AmGmGmG + AmCmUmC + GmAmAmG + GmCmC mU + TmC | |
| 212 | mGmCmCmAmGmGmUmAmAmGmUmAmUmUmU + | 11130.3 |
| | GmAmG mG + GmAmCmU + CmGmAmA + GmGmC mC + TmU | |
| 213 | mGmCmCmAmGmGmUmAmAmGmUmAmUmCmU + | 11143.3 |
| | TmGmAmG + GmGmAmC + TmCmGmA + AmGmGmC + CmU | |
| 214 | mGmCmCmAmGmGmUmAmAmGmUmAmUmAmC + | 11166.4 |
| | TmUmGmA + GmG mGmA + CmUmCmG + AmAmG mG + CmC | |
| 215 | mGmCmCmAmGmGmUmAmAmGmUmAmUmGmA + | 11178.3 |
| | CmUmUmG + AmGmGmG + A mCmUmC + GmAmA | |
| | mG + GmC | |
| 216 | mGmCmCmAmGmGmUmAmAmGmUmAmUmGmG + | 11218.4 |
| | AmCmUmU + GmAmGmG + G mAmCmU + CmGmA | |
| | mA + GmG | |
| 217 | mGmCmCmAmGmGmUmAmAmGmUmAmUmAmG + | 11216.4 |
| | GmAmC mU + TmG mAmG + G mGmAmC + TmCmG | |
| | mA + AmG | |
| 218 | mGmCmCmAmGmGmUmAmAmGmUmAmUmAmA + | 11200.4 |
| | GmGmAmC + TmUmGmA + G mGmGmA + CmUmC mG + AmA | |
| 219 | mGmCmCmAmGmGmUmAmAmGmUmAmUmGmA + | 11202.4 |
| | AmGmGmA + CmUmUmG + AmGmGmG + AmCmU mC + GmA | |
| 220 | mGmCmCmAmGmGmUmAmAmGmUmAmUmGmG + | 11218.4 |
| | AmAmGmG + AmCmUmU + G mAmGmG + GmAmC | |
| | mU + CmG | |
| 221 | mGmCmCmAmGmGmUmAmAmGmUmAmUmUmG + | 11193.3 |
| | GmAmAmG + GmAmCmU + TmGmAmG + GmGmA mC + TmC | |
| 222 | mGmCmCmAmGmGmUmAmAmGmUmAmUmCmU + | 11193.4 |
| | GmGmAmA + GmGmAmC + TmUmGmA + GmGmG mA + CmU | |
| 223 | mGmCmC mAmGmG mUmAmA mGmUmA mUmGmC + | 11232.4 |
| | TmGmG mA + AmG mGmA + C mUmUmG + AmGmG | |
| | mG + AmC | |
| 224 | mGmCmCmAmGmGmUmAmAmGmUmAmUmUmG + | 11219.3 |
| | CmUmG mG + AmAmGmG + A mCmUmU + GmAmG | |
| | mG + GmA | |
| 225 | mGmCmCmAmGmGmUmAmAmGmUmAmUmCmU + | 11195.3 |
| | GmCmUmG + GmAmAmG + GmAmCmU + TmGmA mG + GmG | |
| 226 | mGmCmCmAmGmGmUmAmAmGmUmAmUmGmC + | 11209.3 |
| | TmGmCmU + GmG mAmA + G mGmAmC + TmUmG | |
| | mA + GmG | |
| 227 | mGmCmCmAmGmGmUmAmAmGmUmAmUmUmG + | 11184.3 |
| | CmUmGmC + TmGmGmA + AmGmGmA + CmUmUmG + AmG | |
| 228 | mGmU + CmCmAmU + CmUmUmG + GmAmCmC + C | 10990.2 |
| | mGmUmC + CmCmG mCmCmAmGmGmUmAmAmG | |
| | mUmAmU | |
| 229 | mCmG + G mCmCmG + TmCmC mA + TmC mUmU + G | 11015.2 |
| | mGmAmC + CmCmG mCmCmA mGmGmU mAmAmG | |
| | mUmAmU | |
| 230 | mCmU + G mAmGmC + GmGmCmC + GmUmCmC + A | 11028.2 |
| | mUmCmU + TmGmG mCmCmA mGmGmUmAmAmG | |
| | mUmAmU | |
| 231 | mAmG + AmAmCmC + TmGmAmG + CmGmGmC + C mGmUmC + | 11139.4 |
| | CmAmGmCmCmAmGmGmUmAmAmG mUmAmU | |
| 232 | mAmA + A mGmCmA + GmAmAmC + CmUmGmA + G | 11158.4 |
| | mCmGmG + CmCmG mCmCmAmGmGmUmAmAmG | |
| | mUmAmU | |
| 233 | mAmG + GmUmAmA + AmAmGmC + AmG mAmA + C | 11193.4 |
| | mCmUmG + AmGmGmCmCmAmGmGmU mAmAmG | |
| | mUmAmU | |
| 234 | mGmC + CmGmCmA + GmGmUmA + AmAmAmG + C | 11182.4 |
| | mAmGmA + AmCmG mCmCmAmGmGmUmAmAmG | |
| | mUmAmU | |
| 235 | mUmC + TmGmGmG + CmCmGmC + AmG mGmU + A | 11152.3 |
| | mAmAmA + GmCmGmCmCmA mGmGmU mAmAmG | |
| | mUmAmU | |
| 236 | mUmC + G mCmGmG + CmGmCmC + GmC mUmC + A | 11039.3 |
| | mGmCmA + CmCmGmCmCmAmGmGmUmAmAmG | |
| | mUmAmU | |
| 237 | mCmC + G mAmCmU + CmGmC mG + GmCmGmC + C | 11053.3 |
| | mGmCmU + CmAmG mCmCmA mGmGmUmAmAmG | |
| | mUmAmU | |
| 238 | mCmC + G mGmAmG + GmCmCmU + CmGmGmG + C | 11133.3 |
| | mCmGmA + CmUmGmCmCmAmGmGmUmAmAmG | |
| | mUmAmU | |
| 239 | mCmG + G mCmAmG + TmCmCmC + CmG mGmA + G | 11093.3 |
| | mGmCmC + TmCmGmCmCmAmGmGmU mAmAmG | |
| | mUmAmU | |
| 240 | mCmG + GmCmAmC + GmGmCmA + GmU mCmC + C | 11128.3 |
| | mCmGmG + AmGmGmCmCmA mGmGmU mAmAmG | |
| | mUmAmU | |
| 241 | mGmU + CmUmCmC + CmGmCmC + CmGmGmC + A | 11067.3 |
| | mCmGmG + CmAmG mCmCmA mGmGmUmAmAmG | |
| | mUmAmU | |
| 242 | mGmC + CmAmUmG + GmCmG mG + TmCmUmC + C | 11044.3 |
| | mCmGmC + CmCmG mCmCmA mGmGmUmAmAmG | |
| | mUmAmU | |
| 243 | mGmG + G mUmCmG + CmCmAmU + GmGmCmG + G | 11097.3 |
| | mUmCmU + CmCmG mCmCmAmGmGmUmAmAmG | |
| | mUmAmU | |
| 244 | mUmU + CmCmAmG + GmGmUmC + GmC mCmA + T | 11121.3 |
| | mGmGmC + GmGmGmCmCmAmGmGmU mAmAmG | |
| | mUmAmU | |
| 245 | mAmG + CmUmUmU + TmCmCmA + GmG mGmU + C | 11041.2 |
| | mGmCmC + AmUmGmCmCmAmGmGmU mAmAmG | |
| | mUmAmU | |
| 246 | mUmC + AmUmCmA + GmCmUmU + TmUmCmC + A | 10988.2 |
| | mGmGmG + TmCmGmCmCmAmGmGmUmAmAmG | |
| | mUmAmU | |
| 247 | mGmG + CmCmUmU + CmAmUmC + AmGmCmU + T | 10976.2 |
| | mUmUmC + CmAmGmCmCmAmGmGmUmAmAmG | |
| | mUmAmU | |
| 248 | mUmC + GmAmAmG + GmCmCmU + TmC mAmU + C | 11025.2 |
| | mAmGmC + TmUmGmCmCmAmGmGmU mAmAmG | |
| | mUmAmU | |
| 249 | mGmG + G mAmCmU + CmGmAmA + GmGmCmC + T | 11103.3 |
| | mUmCmA + TmCmGmCmCmAmGmGmUmAmAmG | |
| | mUmAmU | |
| 250 | mCmU + T mGmAmG + GmGmA mC + TmCmGmA + A | 11143.3 |
| | mGmGmC + CmUmGmCmCmAmGmGmUmAmAmG | |
| | mUmAmU | |
| 251 | mAmA + GmGmAmC + TmUmGmA + GmGmGmA + C | 11200.4 |
| | mUmCmG + AmAmGmCmCmAmGmGmUmAmAmG | |
| | mUmAmU | |
| 252 | mGmC + T mGmGmA + AmGmGmA + CmUmUmG + A | 11232.4 |
| | mGmGmG + AmCmGmCmCmA mGmGmUmAmAmG | |
| | mUmAmU | |
| 253 | mGmCmCmAmGmGmUmAmAmGmUmAmUmGmU + | 11063.3 |
| | CmAmGmA + AmAmUmA + CmAmCmA + AmUmAmC + CmU | |
| 254 | mGmU + CmAmGmA + AmAmUmA + CmAmCmA + A | 11063.3 |
| | mUmAmC + CmUmGmCmCmAmGmGmUmAmAmG | |
| | mUmAmU | |

TABLE 2
Exemplary bifunctional oligonucleotides with reference sequences

| SEQ ID | | |
|---|---|---|
| NO. | Reference sequence | Sequence |
| 100 | GCCAGGUAAGUAUA | mGmCmCmAmGmGmUmAmAmGmUmAmUmAmU+C |
| | UCUUGGACCCGUCCC | mUmUmG+GmAmCmC+CmGmUmC+CmCmGmG+Cm |
| | GGCA | A |
| 101 | GCCAGGUAAGUAUC | mGmCmCmAmGmGmUmAmAmGmUmAmUmCmA+T |
| | ATCUUGGACCCGUCC | mCmUmU+GmGmAmC+CmCmGmU+CmCmCmG+Gm |
| | CGGC | C |
| 102 | GCCAGGUAAGUAUC | mGmCmCmAmGmGmUmAmAmGmUmAmUmCmC+A |
| | CAUCUTGGACCCGTC | mUmCmU+TmGmGmA+CmCmCmG+TmCmCmC+Gm |
| | CCGG | G |
| 103 | GCCAGGUAAGUAUU | mGmCmCmAmGmGmUmAmAmGmUmAmUmUmC+C |
| | CCAUCTUGGACCCGU | mAmUmC+TmUmGmG+AmCmCmC+GmUmCmC+Cm |
| | CCCG | G |
| 104 | GCCAGGUAAGUAUG | mGmCmCmAmGmGmUmAmAmGmUmAmUmGmU+C |
| | UCCAUCUUGGACCC | mCmAmU+CmUmUmG+GmAmCmC+CmGmUmC+Cm |
| | GUCCC | C |
| 105 | GCCAGGUAAGUAUC | mGmCmCmAmGmGmUmAmAmGmUmAmUmCmG+T |
| | GTCCATCUUGGACCC | mCmCmA+TmCmUmU+GmGmAmC+CmCmGmU+Cm |
| | GUCC | C |
| 106 | GCCAGGUAAGUAUC | mGmCmCmAmGmGmUmAmAmGmUmAmUmCmC+G |
| | CGUCCAUCUTGGACC | mUmCmC+AmUmCmU+TmGmGmA+CmCmCmG+Tm |
| | CGTC | C |
| 107 | GCCAGGUAAGUAUG | mGmCmCmAmGmGmUmAmAmGmUmAmUmGmC+C |
| | CCGUCCAUCTUGGAC | mGmUmC+CmAmUmC+TmUmGmG+AmCmCmC+Gm |
| | CCGU | U |
| 108 | GCCAGGUAAGUAUG | mGmCmCmAmGmGmUmAmAmGmUmAmUmGmG+C |
| | GCCGUCCAUCUUGG | mCmGmU+CmCmAmU+CmUmUmG+GmAmCmC+Cm |
| | ACCCG | G |
| 109 | GCCAGGUAAGUAUC | mGmCmCmAmGmGmUmAmAmGmUmAmUmCmG+G |
| | GGCCGTCCATCUUGG | mCmCmG+TmCmCmA+TmCmUmU+GmGmAmC+Cm |
| | ACCC | C |
| 110 | GCCAGGUAAGUAUG | mGmCmCmAmGmGmUmAmAmGmUmAmUmGmC+G |
| | CGGCCGUCCAUCUTG | mGmCmC+GmUmCmC+AmUmCmU+TmGmGmA+Cm |
| | GACC | C |
| 111 | GCCAGGUAAGUAUA | mGmCmCmAmGmGmUmAmAmGmUmAmUmAmG+C |
| | GCGGCCGUCCAUCTU | mGmGmC+CmGmUmC+CmAmUmC+TmUmGmG+Am |
| | GGAC | C |
| 112 | GCCAGGUAAGUAUG | mGmCmCmAmGmGmUmAmAmGmUmAmUmGmA+G |
| | AGCGGCCGUCCAUC | mCmGmG+CmCmGmU+CmCmAmU+CmUmUmG+Gm |
| | UUGGA | A |
| 113 | GCCAGGUAAGUAUU | mGmCmCmAmGmGmUmAmAmGmUmAmUmUmG+A |
| | GAGCGGCCGTCCATC | mGmCmG+GmCmCmG+TmCmCmA+TmCmUmU+Gm |
| | UUGG | G |
| 114 | GCCAGGUAAGUAUC | mGmCmCmAmGmGmUmAmAmGmUmAmUmCmU+G |
| | UGAGCGGCCGUCCA | mAmGmC+GmGmCmC+GmUmCmC+AmUmCmU+Tm |
| | UCUTG | G |
| 115 | GCCAGGUAAGUAUC | mGmCmCmAmGmGmUmAmAmGmUmAmUmCmC+T |
| | CTGAGCGGCCGUCCA | mGmAmG+CmGmGmC+CmGmUmC+CmAmUmC+Tm |
| | UCTU | U |
| 116 | GCCAGGUAAGUAUA | mGmCmCmAmGmGmUmAmAmGmUmAmUmAmC+C |
| | CCUGAGCGGCCGUCC | mUmGmA+GmCmGmG+CmCmGmU+CmCmAmU+Cm |
| | AUCU | U |
| 117 | GCCAGGUAAGUAUA | mGmCmCmAmGmGmUmAmAmGmUmAmUmAmA+C |
| | ACCUGAGCGGCCGTC | mCmUmG+AmGmCmG+GmCmCmG+TmCmCmA+Tm |
| | CATC | C |
| 118 | GCCAGGUAAGUAUG | mGmCmCmAmGmGmUmAmAmGmUmAmUmGmA+A |
| | AACCUGAGCGGCCG | mCmCmU+GmAmGmC+GmGmCmC+GmUmCmC+Am |
| | UCCAU | U |
| 119 | GCCAGGUAAGUAUA | mGmCmCmAmGmGmUmAmAmGmUmAmUmAmG+A |
| | GAACCTGAGCGGCCG | mAmCmC+TmGmAmG+CmGmGmC+CmGmUmC+Cm |
| | UCCA | A |
| 120 | GCCAGGUAAGUAUC | mGmCmCmAmGmGmUmAmAmGmUmAmUmCmA+G |
| | AGAACCUGAGCGGC | mAmAmC+CmUmGmA+GmCmGmG+CmCmGmU+Cm |
| | CGUCC | C |
| 121 | GCCAGGUAAGUAUG | mGmCmCmAmGmGmUmAmAmGmUmAmUmGmC+A |
| | CAGAACCUGAGCGG | mGmAmA+CmCmUmG+AmGmCmG+GmCmCmG+Tm |
| | CCGTC | C |
| 122 | GCCAGGUAAGUAUA | mGmCmCmAmGmGmUmAmAmGmUmAmUmAmG+C |
| | GCAGAACCUGAGCG | mAmGmA+AmCmCmU+GmAmGmC+GmGmCmC+Gm |
| | GCCGU | U |
| 123 | GCCAGGUAAGUAUA | mGmCmCmAmGmGmUmAmAmGmUmAmUmAmA+G |
| | AGCAGAACCTGAGC | mCmAmG+AmAmCmC+TmGmAmG+CmGmGmC+Cm |
| | GGCCG | G |
| 124 | GCCAGGUAAGUAUA | mGmCmCmAmGmGmUmAmAmGmUmAmUmAmA+A |
| | AAGCAGAACCUGAG | mGmCmA+GmAmAmC+CmUmGmA+GmCmGmG+Cm |
| | CGGCC | C |
| 125 | GCCAGGUAAGUAUA | mGmCmCmAmGmGmUmAmAmGmUmAmUmAmA+A |
| | AAAGCAGAACCUGA | mAmGmC+AmGmAmA+CmCmUmG+AmGmCmG+G |
| | GCGGC | mC |
| 126 | GCCAGGUAAGUAUU | mGmCmCmAmGmGmUmAmAmGmUmAmUmUmA+A |
| | AAAAGCAGAACCUG | mAmAmG+CmAmGmA+AmCmCmU+GmAmGmC+G |
| | AGCGG | mG |
| 127 | GCCAGGUAAGUAUG | mGmCmCmAmGmGmUmAmAmGmUmAmUmGmU+A |
| | UAAAAGCAGAACCT | mAmAmA+GmCmAmG+AmAmCmC+TmGmAmG+Cm |
| | GAGCG | G |
| 128 | GCCAGGUAAGUAUG | mGmCmCmAmGmGmUmAmAmGmUmAmUmGmG+T |
| | GTAAAAGCAGAACC | mAmAmA+AmGmCmA+GmAmAmC+CmUmGmA+G |
| | UGAGC | mC |
| 129 | GCCAGGUAAGUAUA | mGmCmCmAmGmGmUmAmAmGmUmAmUmAmG+G |
| | GGUAAAAGCAGAAC | mUmAmA+AmAmGmC+AmGmAmA+CmCmUmG+A |
| | CUGAG | mG |
| 130 | GCCAGGUAAGUAUC | mGmCmCmAmGmGmUmAmAmGmUmAmUmCmA+G |
| | AGGUAAAAGCAGAA | mGmUmA+AmAmAmG+CmAmGmA+AmCmCmU+G |
| | CCUGA | mA |
| 131 | GCCAGGUAAGUAUG | mGmCmCmAmGmGmUmAmAmGmUmAmUmGmC+A |
| | CAGGUAAAAGCAGA | mGmGmU+AmAmAmA+GmCmAmG+AmAmCmC+Tm |
| | ACCTG | G |
| 132 | GCCAGGUAAGUAUC | mGmCmCmAmGmGmUmAmAmGmUmAmUmCmG+C |
| | GCAGGTAAAAGCAG | mAmGmG+TmAmAmA+AmGmCmA+GmAmAmC+Cm |
| | AACCU | U |
| 133 | GCCAGGUAAGUAUC | mGmCmCmAmGmGmUmAmAmGmUmAmUmCmC+G |
| | CGCAGGUAAAAGCA | mCmAmG+GmUmAmA+AmAmGmC+AmGmAmA+C |
| | GAACC | mC |
| 134 | GCCAGGUAAGUAUG | mGmCmCmAmGmGmUmAmAmGmUmAmUmGmC+C |
| | CCGCAGGUAAAAGC | mGmCmA+GmGmUmA+AmAmAmG+CmAmGmA+A |
| | AGAAC | mC |
| 135 | GCCAGGUAAGUAUG | mGmCmCmAmGmGmUmAmAmGmUmAmUmGmG+C |
| | GCCGCAGGUAAAAG | mCmGmC+AmGmGmU+AmAmAmA+GmCmAmG+A |
| | CAGAA | mA |
| 136 | GCCAGGUAAGUAUG | mGmCmCmAmGmGmUmAmAmGmUmAmUmGmG+G |
| | GGCCGCAGGTAAAA | mCmCmG+CmAmGmG+TmAmAmA+AmGmCmA+Gm |
| | GCAGA | A |
| 137 | GCCAGGUAAGUAUU | mGmCmCmAmGmGmUmAmAmGmUmAmUmUmG+G |
| | GGGCCGCAGGUAAA | mGmCmC+GmCmAmG+GmUmAmA+AmAmGmC+A |
| | AGCAG | mG |
| 138 | GCCAGGUAAGUAUC | mGmCmCmAmGmGmUmAmAmGmUmAmUmCmU+G |
| | UGGGCCGCAGGUAA | mGmGmC+CmGmCmA+GmGmUmA+AmAmAmG+C |
| | AAGCA | mA |
| 139 | GCCAGGUAAGUAUU | mGmCmCmAmGmGmUmAmAmGmUmAmUmUmC+T |
| | CTGGGCCGCAGGUA | mGmGmG+CmCmGmC+AmGmGmU+AmAmAmA+G |
| | AAAGC | mC |
| 140 | GCCAGGUAAGUAUC | mGmCmCmAmGmGmUmAmAmGmUmAmUmCmU+C |
| | UCUGGGCCGCAGGT | mUmGmG+GmCmCmG+CmAmGmG+TmAmAmA+Am |
| | AAAAG | G |
| 141 | GCCAGGUAAGUAUG | mGmCmCmAmGmGmUmAmAmGmUmAmUmGmC+T |
| | CTCUGGGCCGCAGGU | mCmUmG+GmGmCmC+GmCmAmG+GmUmAmA+A |
| | AAAA | mA |
| 142 | GCCAGGUAAGUAUG | mGmCmCmAmGmGmUmAmAmGmUmAmUmGmG+C |
| | GCUCUGGGCCGCAG | mUmCmU+GmGmGmC+CmGmCmA+GmGmUmA+A |
| | GUAAA | mA |
| 143 | GCCAGGUAAGUAUG | mGmCmCmAmGmGmUmAmAmGmUmAmUmGmG+G |
| | GGCUCTGGGCCGCAG | mCmUmC+TmGmGmG+CmCmGmC+AmGmGmU+Am |
| | GUAA | A |
| 144 | GCCAGGUAAGUAUU | mGmCmCmAmGmGmUmAmAmGmUmAmUmUmC+G |
| | CGCGGCGCCGCUCAG | mCmGmG+CmGmCmC+GmCmUmC+AmGmCmA+Cm |
| | CACC | C |
| 145 | GCCAGGUAAGUAUC | mGmCmCmAmGmGmUmAmAmGmUmAmUmCmU+C |
| | UCGCGGCGCCGCUCA | mGmCmG+GmCmGmC+CmGmCmU+CmAmGmC+Am |
| | GCAC | C |
| 146 | GCCAGGUAAGUAUA | mGmCmCmAmGmGmUmAmAmGmUmAmUmAmC+T |
| | CTCGCGGCGCCGCTC | mCmGmC+GmGmCmG+CmCmGmC+TmCmAmG+Cm |
| | AGCA | A |
| 147 | GCCAGGUAAGUAUG | mGmCmCmAmGmGmUmAmAmGmUmAmUmGmA+C |
| | ACUCGCGGCGCCGCU | mUmCmG+CmGmGmC+GmCmCmG+CmUmCmA+Gm |
| | CAGC | C |
| 148 | GCCAGGUAAGUAUC | mGmCmCmAmGmGmUmAmAmGmUmAmUmCmG+A |
| | GACUCGCGGCGCCGC | mCmUmC+GmCmGmG+CmGmCmC+GmCmUmC+Am |
| | UCAG | G |
| 149 | GCCAGGUAAGUAUC | mGmCmCmAmGmGmUmAmAmGmUmAmUmCmC+G |
| | CGACUCGCGGCGCCG | mAmCmU+CmGmCmG+GmCmGmC+CmGmCmU+Cm |
| | CUCA | A |
| 150 | GCCAGGUAAGUAUA | mGmCmCmAmGmGmUmAmAmGmUmAmUmAmG+G |
| | GGCCUCGGGCCGAC | mCmCmU+CmGmGmG+CmCmGmA+CmUmCmG+Cm |
| | UCGCG | G |
| 151 | GCCAGGUAAGUAUG | mGmCmCmAmGmGmUmAmAmGmUmAmUmGmA+G |
| | AGGCCTCGGGCCGAC | mGmCmC+TmCmGmG+GmCmCmG+AmCmUmC+Gm |
| | UCGC | C |
| 152 | GCCAGGUAAGUAUG | mGmCmCmAmGmGmUmAmAmGmUmAmUmGmG+A |
| | GAGGCCUCGGGCCG | mGmGmC+CmUmCmG+GmGmCmC+GmAmCmU+Cm |
| | ACUCG | G |
| 153 | GCCAGGUAAGUAUC | mGmCmCmAmGmGmUmAmAmGmUmAmUmCmG+G |
| | GGAGGCCUCGGGCC | mAmGmG+CmCmUmC+GmGmGmC+CmGmAmC+Tm |
| | GACTC | C |
| 154 | GCCAGGUAAGUAUC | mGmCmCmAmGmGmUmAmAmGmUmAmUmCmC+G |
| | CGGAGGCCUCGGGC | mGmAmG+GmCmCmU+CmGmGmG+CmCmGmA+Cm |
| | CGACU | U |
| 155 | GCCAGGUAAGUAUA | mGmCmCmAmGmGmUmAmAmGmUmAmUmAmG+T |
| | GTCCCCGGAGGCCTC | mCmCmC+CmGmGmA+GmGmCmC+TmCmGmG+Gm |
| | GGGC | C |
| 156 | GCCAGGUAAGUAUC | mGmCmCmAmGmGmUmAmAmGmUmAmUmCmA+G |
| | AGUCCCCGGAGGCC | mUmCmC+CmCmGmG+AmGmGmC+CmUmCmG+Gm |
| | UCGGG | G |
| 157 | GCCAGGUAAGUAUG | mGmCmCmAmGmGmUmAmAmGmUmAmUmGmC+A |
| | CAGUCCCCGGAGGCC | mGmUmC+CmCmCmG+GmAmGmG+CmCmUmC+Gm |
| | UCGG | G |
| 158 | GCCAGGUAAGUAUG | mGmCmCmAmGmGmUmAmAmGmUmAmUmGmG+C |
| | GCAGUCCCCGGAGG | mAmGmU+CmCmCmC+GmGmAmG+GmCmCmU+Cm |
| | CCUCG | G |
| 159 | GCCAGGUAAGUAUC | mGmCmCmAmGmGmUmAmAmGmUmAmUmCmG+G |
| | GGCAGTCCCCGGAGG | mCmAmG+TmCmCmC+CmGmGmA+GmGmCmC+Tm |
| | CCTC | C |
| 160 | GCCAGGUAAGUAUA | mGmCmCmAmGmGmUmAmAmGmUmAmUmAmC+G |
| | CGGCAGUCCCCGGA | mGmCmA+GmUmCmC+CmCmGmG+AmGmGmC+Cm |
| | GGCCU | U |
| 161 | GCCAGGUAAGUAUC | mGmCmCmAmGmGmUmAmAmGmUmAmUmCmA+C |
| | ACGGCAGUCCCCGG | mGmGmC+AmGmUmC+CmCmCmG+GmAmGmG+Cm |
| | AGGCC | C |
| 162 | GCCAGGUAAGUAUG | mGmCmCmAmGmGmUmAmAmGmUmAmUmGmC+A |
| | CACGGCAGUCCCCGG | mCmGmG+CmAmGmU+CmCmCmC+GmGmAmG+Gm |
| | AGGC | C |
| 163 | GCCAGGUAAGUAUG | mGmCmCmAmGmGmUmAmAmGmUmAmUmGmG+C |
| | GCACGGCAGTCCCCG | mAmCmG+GmCmAmG+TmCmCmC+CmGmGmA+Gm |
| | GAGG | G |
| 164 | GCCAGGUAAGUAUC | mGmCmCmAmGmGmUmAmAmGmUmAmUmCmG+G |
| | GGCACGGCAGUCCCC | mCmAmC+GmGmCmA+GmUmCmC+CmCmGmG+Am |
| | GGAG | G |
| 165 | GCCAGGUAAGUAUC | mGmCmCmAmGmGmUmAmAmGmUmAmUmCmC+G |
| | CGGCACGGCAGUCCC | mGmCmA+CmGmGmC+AmGmUmC+CmCmCmG+Gm |
| | CGGA | A |
| 166 | GCCAGGUAAGUAUU | mGmCmCmAmGmGmUmAmAmGmUmAmUmUmC+C |
| | CCCGCCCGGCACGGC | mCmGmC+CmCmGmG+CmAmCmG+GmCmAmG+Tm |
| | AGTC | C |
| 167 | GCCAGGUAAGUAUC | mGmCmCmAmGmGmUmAmAmGmUmAmUmCmU+C |
| | UCCCGCCCGGCACGG | mCmCmG+CmCmCmG+GmCmAmC+GmGmCmA+Gm |
| | CAGU | U |
| 168 | GCCAGGUAAGUAUU | mGmCmCmAmGmGmUmAmAmGmUmAmUmUmC+T |
| | CTCCCGCCCGGCACG | mCmCmC+GmCmCmC+GmGmCmA+CmGmGmC+Am |
| | GCAG | G |
| 169 | GCCAGGUAAGUAUG | mGmCmCmAmGmGmUmAmAmGmUmAmUmGmU+C |
| | UCUCCCGCCCGGCAC | mUmCmC+CmGmCmC+CmGmGmC+AmCmGmG+Cm |
| | GGCA | A |
| 170 | GCCAGGUAAGUAUU | mGmCmCmAmGmGmUmAmAmGmUmAmUmUmG+G |
| | GGCGGTCUCCCGCCC | mCmGmG+TmCmUmC+CmCmGmC+CmCmGmG+Cm |
| | GGCA | A |
| 171 | GCCAGGUAAGUAUA | mGmCmCmAmGmGmUmAmAmGmUmAmUmAmU+G |
| | UGGCGGUCUCCCGCC | mGmCmG+GmUmCmU+CmCmCmG+CmCmCmG+Gm |
| | CGGC | C |
| 172 | GCCAGGUAAGUAUC | mGmCmCmAmGmGmUmAmAmGmUmAmUmCmA+T |
| | ATGGCGGUCTCCCGC | mGmGmC+GmGmUmC+TmCmCmC+GmCmCmC+Gm |
| | CCGG | G |
| 173 | GCCAGGUAAGUAUC | mGmCmCmAmGmGmUmAmAmGmUmAmUmCmC+A |
| | CAUGGCGGUCUCCC | mUmGmG+CmGmGmU+CmUmCmC+CmGmCmC+Cm |
| | GCCCG | G |
| 174 | GCCAGGUAAGUAUG | mGmCmCmAmGmGmUmAmAmGmUmAmUmGmC+C |
| | CCAUGGCGGTCUCCC | mAmUmG+GmCmGmG+TmCmUmC+CmCmGmC+Cm |
| | GCCC | C |
| 175 | GCCAGGUAAGUAUC | mGmCmCmAmGmGmUmAmAmGmUmAmUmCmG+C |
| | GCCAUGGCGGUCUC | mCmAmU+GmGmCmG+GmUmCmU+CmCmCmG+Cm |
| | CCGCC | C |
| 176 | GCCAGGUAAGUAUU | mGmCmCmAmGmGmUmAmAmGmUmAmUmUmC+G |
| | CGCCATGGCGGUCTC | mCmCmA+TmGmGmC+GmGmUmC+TmCmCmC+Gm |
| | CCGC | C |
| 177 | GCCAGGUAAGUAUG | mGmCmCmAmGmGmUmAmAmGmUmAmUmGmU+C |
| | UCGCCAUGGCGGUC | mGmCmC+AmUmGmG+CmGmGmU+CmUmCmC+Cm |
| | UCCCG | G |
| 178 | GCCAGGUAAGUAUG | mGmCmCmAmGmGmUmAmAmGmUmAmUmGmG+T |
| | GTCGCCAUGGCGGTC | mCmGmC+CmAmUmG+GmCmGmG+TmCmUmC+Cm |
| | UCCC | C |
| 179 | GCCAGGUAAGUAUG | mGmCmCmAmGmGmUmAmAmGmUmAmUmGmG+G |
| | GGUCGCCAUGGCGG | mUmCmG+CmCmAmU+GmGmCmG+GmUmCmU+Cm |
| | UCUCC | C |
| 180 | GCCAGGUAAGUAUA | mGmCmCmAmGmGmUmAmAmGmUmAmUmAmG+G |
| | GGGUCGCCATGGCG | mGmUmC+GmCmCmA+TmGmGmC+GmGmUmC+Tm |
| | GUCTC | C |
| 181 | GCCAGGUAAGUAUC | mGmCmCmAmGmGmUmAmAmGmUmAmUmCmA+G |
| | AGGGUCGCCAUGGC | mGmGmU+CmGmCmC+AmUmGmG+CmGmGmU+Cm |
| | GGUCU | U |
| 182 | GCCAGGUAAGUAUC | mGmCmCmAmGmGmUmAmAmGmUmAmUmCmC+A |
| | CAGGGTCGCCAUGGC | mGmGmG+TmCmGmC+CmAmUmG+GmCmGmG+Tm |
| | GGTC | C |
| 183 | GCCAGGUAAGUAUU | mGmCmCmAmGmGmUmAmAmGmUmAmUmUmC+C |
| | CCAGGGUCGCCAUG | mAmGmG+GmUmCmG+CmCmAmU+GmGmCmG+G |
| | GCGGU | mU |
| 184 | GCCAGGUAAGUAUU | mGmCmCmAmGmGmUmAmAmGmUmAmUmUmU+C |
| | UCCAGGGUCGCCATG | mCmAmG+GmGmUmC+GmCmCmA+TmGmGmC+Gm |
| | GCGG | G |
| 185 | GCCAGGUAAGUAUU | mGmCmCmAmGmGmUmAmAmGmUmAmUmUmU+T |
| | UTCCAGGGUCGCCAU | mCmCmA+GmGmGmU+CmGmCmC+AmUmGmG+Cm |
| | GGCG | G |
| 186 | GCCAGGUAAGUAUU | mGmCmCmAmGmGmUmAmAmGmUmAmUmUmU+T |
| | UTUCCAGGGTCGCCA | mUmCmC+AmGmGmG+TmCmGmC+CmAmUmG+Gm |
| | UGGC | C |
| 187 | GCCAGGUAAGUAUC | mGmCmCmAmGmGmUmAmAmGmUmAmUmCmU+T |
| | UTUUCCAGGGUCGCC | mUmUmC+CmAmGmG+GmUmCmG+CmCmAmU+Gm |
| | AUGG | G |
| 188 | GCCAGGUAAGUAUG | mGmCmCmAmGmGmUmAmAmGmUmAmUmGmC+T |
| | CTUUUCCAGGGUCGC | mUmUmU+CmCmAmG+GmGmUmC+GmCmCmA+Tm |
| | CATG | G |
| 189 | GCCAGGUAAGUAUA | mGmCmCmAmGmGmUmAmAmGmUmAmUmAmG+C |
| | GCUUUTCCAGGGUC | mUmUmU+TmCmCmA+GmGmGmU+CmGmCmC+Am |
| | GCCAU | U |
| 190 | GCCAGGUAAGUAUC | mGmCmCmAmGmGmUmAmAmGmUmAmUmCmA+G |
| | AGCUUTUCCAGGGTC | mCmUmU+TmUmCmC+AmGmGmG+TmCmGmC+Cm |
| | GCCA | A |
| 191 | GCCAGGUAAGUAUU | mGmCmCmAmGmGmUmAmAmGmUmAmUmUmC+A |
| | CAGCUTUUCCAGGG | mGmCmU+TmUmUmC+CmAmGmG+GmUmCmG+Cm |
| | UCGCC | C |
| 192 | GCCAGGUAAGUAUA | mGmCmCmAmGmGmUmAmAmGmUmAmUmAmU+C |
| | UCAGCTUUUCCAGG | mAmGmC+TmUmUmU+CmCmAmG+GmGmUmC+Gm |
| | GUCGC | C |
| 193 | GCCAGGUAAGUAUC | mGmCmCmAmGmGmUmAmAmGmUmAmUmCmA+T |
| | ATCAGCUUUTCCAGG | mCmAmG+CmUmUmU+TmCmCmA+GmGmGmU+Cm |
| | GUCG | G |
| 194 | GCCAGGUAAGUAUU | mGmCmCmAmGmGmUmAmAmGmUmAmUmUmC+A |
| | CAUCAGCUUTUCCAG | mUmCmA+GmCmUmU+TmUmCmC+AmGmGmG+Tm |
| | GGTC | C |
| 195 | GCCAGGUAAGUAUU | mGmCmCmAmGmGmUmAmAmGmUmAmUmUmU+C |
| | UCAUCAGCUTUUCCA | mAmUmC+AmGmCmU+TmUmUmC+CmAmGmG+Gm |
| | GGGU | U |
| 196 | GCCAGGUAAGUAUC | mGmCmCmAmGmGmUmAmAmGmUmAmUmCmU+T |
| | UTCAUCAGCTUUUCC | mCmAmU+CmAmGmC+TmUmUmU+CmCmAmG+Gm |
| | AGGG | G |
| 197 | GCCAGGUAAGUAUC | mGmCmCmAmGmGmUmAmAmGmUmAmUmCmC+T |
| | CTUCATCAGCUUUTC | mUmCmA+TmCmAmG+CmUmUmU+TmCmCmA+Gm |
| | CAGG | G |
| 198 | GCCAGGUAAGUAUG | mGmCmCmAmGmGmUmAmAmGmUmAmUmGmC+C |
| | CCUUCAUCAGCUUTU | mUmUmC+AmUmCmA+GmCmUmU+TmUmCmC+Am |
| | CCAG | G |
| 199 | GCCAGGUAAGUAUG | mGmCmCmAmGmGmUmAmAmGmUmAmUmGmG+C |
| | GCCUUCAUCAGCUTU | mCmUmU+CmAmUmC+AmGmCmU+TmUmUmC+Cm |
| | UCCA | A |
| 200 | GCCAGGUAAGUAUA | mGmCmCmAmGmGmUmAmAmGmUmAmUmAmG+G |
| | GGCCUTCAUCAGCTU | mCmCmU+TmCmAmU+CmAmGmC+TmUmUmU+Cm |
| | UUCC | C |
| 201 | GCCAGGUAAGUAUA | mGmCmCmAmGmGmUmAmAmGmUmAmUmAmA+G |
| | AGGCCTUCATCAGCU | mGmCmC+TmUmCmA+TmCmAmG+CmUmUmU+Tm |
| | UUTC | C |
| 202 | GCCAGGUAAGUAUG | mGmCmCmAmGmGmUmAmAmGmUmAmUmGmA+A |
| | AAGGCCUUCAUCAG | mGmGmC+CmUmUmC+AmUmCmA+GmCmUmU+Tm |
| | CUUTU | U |
| 203 | GCCAGGUAAGUAUC | mGmCmCmAmGmGmUmAmAmGmUmAmUmCmG+A |
| | GAAGGCCUUCAUCA | mAmGmG+CmCmUmU+CmAmUmC+AmGmCmU+Tm |
| | GCUTU | U |
| 204 | GCCAGGUAAGUAUU | mGmCmCmAmGmGmUmAmAmGmUmAmUmUmC+G |
| | CGAAGGCCUTCAUCA | mAmAmG+GmCmCmU+TmCmAmU+CmAmGmC+Tm |
| | GCTU | U |
| 205 | GCCAGGUAAGUAUA | mGmCmCmAmGmGmUmAmAmGmUmAmUmAmC+T |
| | CTCGAAGGCCUUCAU | mCmGmA+AmGmGmC+CmUmUmC+AmUmCmA+Gm |
| | CAGC | C |
| 206 | GCCAGGUAAGUAUG | mGmCmCmAmGmGmUmAmAmGmUmAmUmGmA+C |
| | ACUCGAAGGCCUUC | mUmCmG+AmAmGmG+CmCmUmU+CmAmUmC+Am |
| | AUCAG | G |
| 207 | GCCAGGUAAGUAUG | mGmCmCmAmGmGmUmAmAmGmUmAmUmGmG+A |
| | GACUCGAAGGCCUTC | mCmUmC+GmAmAmG+GmCmCmU+TmCmAmU+Cm |
| | AUCA | A |
| 20 | GCCAGGUAAGUAUG | mGmCmCmAmGmGmUmAmAmGmUmAmUmGmG+G |
| | GGACUCGAAGGCCT | mAmCmU+CmGmAmA+GmGmCmC+TmUmCmA+Tm |
| | UCATC | C |
| 209 | GCCAGGUAAGUAUA | mGmCmCmAmGmGmUmAmAmGmUmAmUmAmG+G |
| | GGGACTCGAAGGCC | mGmAmC+TmCmGmA+AmGmGmC+CmUmUmC+Am |
| | UUCAU | U |
| 210 | GCCAGGUAAGUAUG | mGmCmCmAmGmGmUmAmAmGmUmAmUmGmA+G |
| | AGGGACUCGAAGGC | mGmGmA+CmUmCmG+AmAmGmG+CmCmUmU+Cm |
| | CUUCA | A |
| 211 | GCCAGGUAAGUAUU | mGmCmCmAmGmGmUmAmAmGmUmAmUmUmG+A |
| | GAGGGACUCGAAGG | mGmGmG+AmCmUmC+GmAmAmG+GmCmCmU+Tm |
| | CCUTC | C |
| 212 | GCCAGGUAAGUAUU | mGmCmCmAmGmGmUmAmAmGmUmAmUmUmU+G |
| | UGAGGGACUCGAAG | mAmGmG+GmAmCmU+CmGmAmA+GmGmCmC+Tm |
| | GCCTU | U |
| 213 | GCCAGGUAAGUAUC | mGmCmCmAmGmGmUmAmAmGmUmAmUmCmU+T |
| | UTGAGGGACTCGAA | mGmAmG+GmGmAmC+TmCmGmA+AmGmGmC+Cm |
| | GGCCU | U |
| 214 | GCCAGGUAAGUAUA | mGmCmCmAmGmGmUmAmAmGmUmAmUmAmC+T |
| | CTUGAGGGACUCGA | mUmGmA+GmGmGmA+CmUmCmG+AmAmGmG+C |
| | AGGCC | mC |
| 215 | GCCAGGUAAGUAUG | mGmCmCmAmGmGmUmAmAmGmUmAmUmGmA+C |
| | ACUUGAGGGACUCG | mUmUmG+AmGmGmG+AmCmUmC+GmAmAmG+G |
| | AAGGC | mC |
| 216 | GCCAGGUAAGUAUG | mGmCmCmAmGmGmUmAmAmGmUmAmUmGmG+A |
| | GACUUGAGGGACUC | mCmUmU+GmAmGmG+GmAmCmU+CmGmAmA+G |
| | GAAGG | mG |
| 217 | GCCAGGUAAGUAUA | mGmCmCmAmGmGmUmAmAmGmUmAmUmAmG+G |
| | GGACUTGAGGGACTC | mAmCmU+TmGmAmG+GmGmAmC+TmCmGmA+Am |
| | GAAG | G |
| 218 | GCCAGGUAAGUAUA | mGmCmCmAmGmGmUmAmAmGmUmAmUmAmA+G |
| | AGGACTUGAGGGAC | mGmAmC+TmUmGmA+GmGmGmA+CmUmCmG+Am |
| | UCGAA | A |
| 219 | GCCAGGUAAGUAUG | mGmCmCmAmGmGmUmAmAmGmUmAmUmGmA+A |
| | AAGGACUUGAGGGA | mGmGmA+CmUmUmG+AmGmGmG+AmCmUmC+G |
| | CUCGA | mA |
| 220 | GCCAGGUAAGUAUG | mGmCmCmAmGmGmUmAmAmGmUmAmUmGmG+A |
| | GAAGGACUUGAGGG | mAmGmG+AmCmUmU+GmAmGmG+GmAmCmU+C |
| | ACUCG | mG |
| 221 | GCCAGGUAAGUAUU | mGmCmCmAmGmGmUmAmAmGmUmAmUmUmG+G |
| | GGAAGGACUTGAGG | mAmAmG+GmAmCmU+TmGmAmG+GmGmAmC+Tm |
| | GACTC | C |
| 222 | GCCAGGUAAGUAUC | mGmCmCmAmGmGmUmAmAmGmUmAmUmCmU+G |
| | UGGAAGGACTUGAG | mGmAmA+GmGmAmC+TmUmGmA+GmGmGmA+C |
| | GGACU | mU |
| 223 | GCCAGGUAAGUAUG | mGmCmCmAmGmGmUmAmAmGmUmAmUmGmC+T |
| | CTGGAAGGACUUGA | mGmGmA+AmGmGmA+CmUmUmG+AmGmGmG+A |
| | GGGAC | mC |
| 224 | GCCAGGUAAGUAUU | mGmCmCmAmGmGmUmAmAmGmUmAmUmUmG+C |
| | GCUGGAAGGACUUG | mUmGmG+AmAmGmG+AmCmUmU+GmAmGmG+G |
| | AGGGA | mA |
| 225 | GCCAGGUAAGUAUC | mGmCmCmAmGmGmUmAmAmGmUmAmUmCmU+G |
| | UGCUGGAAGGACUT | mCmUmG+GmAmAmG+GmAmCmU+TmGmAmG+G |
| | GAGGG | mG |
| 226 | GCCAGGUAAGUAUG | mGmCmCmAmGmGmUmAmAmGmUmAmUmGmC+T |
| | CTGCUGGAAGGACTU | mGmCmU+GmGmAmA+GmGmAmC+TmUmGmA+G |
| | GAGG | mG |
| 227 | GCCAGGUAAGUAUU | mGmCmCmAmGmGmUmAmAmGmUmAmUmUmG+C |
| | GCUGCTGGAAGGAC | mUmGmC+TmGmGmA+AmGmGmA+CmUmUmG+A |
| | UUGAG | mG |
| 228 | GUCCAUCUUGGACC | mGmU+CmCmAmU+CmUmUmG+GmAmCmC+CmGm |
| | CGUCCCGCCAGGUA | UmC+CmCmGmCmCmAmGmGmUmAmAmGmUmAm |
| | AGUAU | U |
| 229 | CGGCCGTCCATCUUG | mCmG+GmCmCmG+TmCmCmA+TmCmUmU+GmGm |
| | GACCCGCCAGGUAA | AmC+CmCmGmCmCmAmGmGmUmAmAmGmUmAm |
| | GUAU | U |
| 230 | CUGAGCGGCCGUCC | mCmU+GmAmGmC+GmGmCmC+GmUmCmC+AmUm |
| | AUCUTGGCCAGGUA | CmU+TmGmGmCmCmAmGmGmUmAmAmGmUmAm |
| | AGUAU | U |
| 231 | AGAACCTGAGCGGCC | mAmG+AmAmCmC+TmGmAmG+CmGmGmC+CmGm |
| | GUCCAGCCAGGUAA | UmC+CmAmGmCmCmAmGmGmUmAmAmGmUmAm |
| | GUAU | U |
| 232 | AAAGCAGAACCUGA | mAmA+AmGmCmA+GmAmAmC+CmUmGmA+GmC |
| | GCGGCCGCCAGGUA | mGmG+CmCmGmCmCmAmGmGmUmAmAmGmUmA |
| | AGUAU | mU |
| 233 | AGGUAAAAGCAGAA | mAmG+GmUmAmA+AmAmGmC+AmGmAmA+CmC |
| | CCUGAGGCCAGGUA | mUmG+AmGmGmCmCmAmGmGmUmAmAmGmUmA |
| | AGUAU | mU |
| 234 | GCCGCAGGUAAAAG | mGmC+CmGmCmA+GmGmUmA+AmAmAmG+CmA |
| | CAGAACGCCAGGUA | mGmA+AmCmGmCmCmAmGmGmUmAmAmGmUmA |
| | AGUAU | mU |
| 235 | UCTGGGCCGCAGGU | mUmC+TmGmGmG+CmCmGmC+AmGmGmU+AmAm |
| | AAAAGCGCCAGGUA | AmA+GmCmGmCmCmAmGmGmUmAmAmGmUmAm |
| | AGUAU | U |
| 236 | UCGCGGCGCCGCUCA | mUmC+GmCmGmG+CmGmCmC+GmCmUmC+AmGm |
| | GCACCGCCAGGUAA | CmA+CmCmGmCmCmAmGmGmUmAmAmGmUmAm |
| | GUAU | U |
| 237 | CCGACUCGCGGCGCC | mCmC+GmAmCmU+CmGmCmG+GmCmGmC+CmGm |
| | GCUCAGCCAGGUAA | CmU+CmAmGmCmCmAmGmGmUmAmAmGmUmAm |
| | GUAU | U |
| 238 | CCGGAGGCCUCGGG | mCmC+GmGmAmG+GmCmCmU+CmGmGmG+CmCm |
| | CCGACUGCCAGGUA | GmA+CmUmGmCmCmAmGmGmUmAmAmGmUmAm |
| | AGUAU | U |
| 239 | CGGCAGTCCCCGGAG | mCmG+GmCmAmG+TmCmCmC+CmGmGmA+GmGm |
| | GCCTCGCCAGGUAAG | CmC+TmCmGmCmCmAmGmGmUmAmAmGmUmAm |
| | UAU | U |
| 240 | CGGCACGGCAGUCCC | mCmG+GmCmAmC+GmGmCmA+GmUmCmC+CmCm |
| | CGGAGGCCAGGUAA | GmG+AmGmGmCmCmAmGmGmUmAmAmGmUmAm |
| | GUAU | U |
| 241 | GUCUCCCGCCCGGCA | mGmU+CmUmCmC+CmGmCmC+CmGmGmC+AmCm |
| | CGGCAGCCAGGUAA | GmG+CmAmGmCmCmAmGmGmUmAmAmGmUmAm |
| | GUAU | U |
| 242 | GCCAUGGCGGTCUCC | mGmC+CmAmUmG+GmCmGmG+TmCmUmC+CmCm |
| | CGCCCGCCAGGUAA | GmC+CmCmGmCmCmAmGmGmUmAmAmGmUmAm |
| | GUAU | U |
| 243 | GGGUCGCCAUGGCG | mGmG+GmUmCmG+CmCmAmU+GmGmCmG+GmU |
| | GUCUCCGCCAGGUA | mCmU+CmCmGmCmCmAmGmGmUmAmAmGmUmA |
| | AGUAU | mU |
| 244 | UUCCAGGGUCGCCAT | mUmU+CmCmAmG+GmGmUmC+GmCmCmA+TmGm |
| | GGCGGGCCAGGUAA | GmC+GmGmGmCmCmAmGmGmUmAmAmGmUmAm |
| | GUAU | U |
| 245 | AGCUUUTCCAGGGU | mAmG+CmUmUmU+TmCmCmA+GmGmGmU+CmGm |
| | CGCCAUGCCAGGUA | CmC+AmUmGmCmCmAmGmGmUmAmAmGmUmAm |
| | AGUAU | U |
| 246 | UCAUCAGCUUTUCCA | mUmC+AmUmCmA+GmCmUmU+TmUmCmC+AmGm |
| | GGGTCGCCAGGUAA | GmG+TmCmGmCmCmAmGmGmUmAmAmGmUmAm |
| | GUAU | U |
| 247 | GGCCUUCAUCAGCUT | mGmG+CmCmUmU+CmAmUmC+AmGmCmU+TmUm |
| | UUCCAGCCAGGUAA | UmC+CmAmGmCmCmAmGmGmUmAmAmGmUmAm |
| | GUAU | U |
| 248 | UCGAAGGCCUTCAUC | mUmC+GmAmAmG+GmCmCmU+TmCmAmU+CmAm |
| | AGCTUGCCAGGUAA | GmC+TmUmGmCmCmAmGmGmUmAmAmGmUmAm |
| | GUAU | U |
| 249 | GGGACUCGAAGGCC | mGmG+GmAmCmU+CmGmAmA+GmGmCmC+TmUm |
| | TUCATCGCCAGGUAA | CmA+TmCmGmCmCmAmGmGmUmAmAmGmUmAm |
| | GUAU | U |
| 250 | CUTGAGGGACTCGAA | mCmU+TmGmAmG+GmGmAmC+TmCmGmA+AmGm |
| | GGCCUGCCAGGUAA | GmC+CmUmGmCmCmAmGmGmUmAmAmGmUmAm |
| | GUAU | U |
| 251 | AAGGACTUGAGGGA | mAmA+GmGmAmC+TmUmGmA+GmGmGmA+CmU |
| | CUCGAAGCCAGGUA | mCmG+AmAmGmCmCmAmGmGmUmAmAmGmUmA |
| | AGUAU | mU |
| 252 | GCTGGAAGGACUUG | mGmC+TmGmGmA+AmGmGmA+CmUmUmG+AmG |
| | AGGGACGCCAGGUA | mGmG+AmCmGmCmCmAmGmGmUmAmAmGmUmA |
| | AGUAU | mU |
| 253 | GCCAGGUAAGUAUG | mGmCmCmAmGmGmUmAmAmGmUmAmUmGmU+C |
| | UCAGAAAUACACAA | mAmGmA+AmAmUmA+CmAmCmA+AmUmAmC+C |
| | UACCU | mU |
| 254 | GUCAGAAAUACACA | mGmU+CmAmGmA+AmAmUmA+CmAmCmA+AmU |
| | AUACCUGCCAGGUA | mAmC+CmUmGmCmCmAmGmGmUmAmAmGmUmA |
| | AGUAU | mU |

In an embodiment, the bifunctional oligonucleotide has a sequence selected from a sequence provided in Table 3. In Tables 3, an “in” placed before a nucleotide refers to a 2′OMe modification, a “+” placed before a nucleotide refers to a locked nucleic acid (LNA), a “i2MOE” placed before a nucleotide refers to a 2′-O-methoxy ethyl modification, a “*” placed before a nucleotide refers to a phosphothiorate modification, and a “32MOE” placed before a terminal nucleotide refers to 2′, 3′-O-methoxy ethyl modification. In an embodiment, the bifunctional oligonucleotide comprises a sequence with at least 75%, 80%, 85%, 90%, 95%, 99% or more sequence identity with a nucleotide selected from SEQ TD NOs: 255-294, or a variant or fragment thereof. In an embodiment, the bifunctional oligonucleotide comprises the sequence GCCAGGUAAGUAU (SEQ TD NO: 004). In an embodiment, the bifunctional oligonucleotide comprises a sequence with at least 75%, 80%, 85%, 90%, 95%, 99%, or more sequence identity with SEQ TD NO: 004, or a variant or fragment thereof.

TABLE 3
Exemplary bifunctional oligonucleotides with reference sequences

| SEQ ID | | |
|---|---|---|
| NO. | Reference sequence | Sequence |
| 255 | GCCAGGUAAGUAUA | mG*mC*mC*mA*mGmGmUmAmAmGmUmAmUmAm |
| | AAAGCAGAACCUGA | A+AmAmGmC+AmGmAmA+CmCmUmG+AmGmCmG |
| | GCGGC | +GmC |
| 256 | GCCAGGUAAGUAUA | mGmCmCmAmGmGmUmAmAmGmUmAmUmAmA+A |
| | AAAGCAGAACCUGA | mAmGmC+AmGmAmA+CmCmUmG+AmG*mC*mG*+ |
| | GCGGC | G*mC |
| 257 | GCCAGGUAAGUAUA | mG*mC*mC*mA*mGmGmUmAmAmGmUmAmUmAm |
| | AAAGCAGAACCUGA | A+AmAmGmC+AmGmAmA+CmCmUmG+AmG*mC* |
| | GCGGC | mG*+G*mC |
| 258 | GCCAGGUAAGUAUA | mGmCmCmAmGmGmUmAmAmGmUmAmU/i2MOErA |
| | AAAGCAGAACCUGA | //i2MOErA/+A/i2MOErA//i2MOErG//i2MOErC/+A/ |
| | GCGGC | i2MOErG//i2MOErA//i2MOErA/+C/i2MOErC//ii2MOErT// |
| | | i2MOErG/+A/i2MOErG/*/i2MOErC/*/i2MOErG/*+G*/ |
| | | 32MOErC/ |
| 259 | GCCAGGUAAGUAUA | mG*mC*mC*mA*mGmGmUmAmAmGmUmAmU/ |
| | AAAGCAGAACCUGA | i2MOErA//i2MOErA/+A/i2MOErA//i2MOErG//i2MOErC/ |
| | GCGGC | +A/i2MOErG//i2MOErA//i2MOErA/+C/i2MOErC// |
| | | i2MOErT//i2MOErG/+A/i2MOErG/*/i2MOErC/*/i2MOErG/*+G |
| | | */32MOErC/ |
| 260 | GCCAGGUAAGUAUA | /52MOErG/*+C*/i2MOErC/*+A*/i2MOErG/+G/ |
| | AAAGCAGAACCUGA | i2MOErT/+A/i2MOErA/+G/i2MOErT//i2MOErA//i2MOErT/ |
| | GCGGC | mAmA+AmAmGmC+AmGmAmA+CmCmUmG+AmGmCm |
| | | G+GmC |
| 261 | GCCAGGUAAGUAUA | /52MOErG/*+C*/i2MOErC/*+A*/i2MOErG/+G/ |
| | AAAGCAGAACCUGA | i2MOErT/+A/i2MOErA/+G/i2MOErT//i2MOErA//i2MOErT/ |
| | GCGGC | mAmA+AmAmGmC+AmGmAmA+CmCmUmG+AmG*mC |
| | | *mG*+G*mC |
| 262 | GCCAGGUAAGUAUA | /52MOErG/*+C*/i2MOErC/*+A*/i2MOErG/+G/ |
| | AAAGCAGAACCUGA | i2MOErT/+A/i2MOErA/+G/i2MOErT//i2MOErA//i2MOErT// |
| | GCGGC | i2MOErA//i2MOErA/+A/i2MOErA//i2MOErG//i2MOErC/ |
| | | +A/i2MOErG//i2MOErA//i2MOErA/+C/i2MOErC// |
| | | i2MOErT//i2MOErG/+A/i2MOErG/*/i2MOErC/*/i2MOErG/ |
| | | *+G*/32MOErC/ |
| 263 | GCCAGGUAAGUAUU | mG*mC*mC*mA*mGmGmUmAmAmGmUmAmUmUm |
| | UCCAGGGUCGCCAT | U+CmCmAmG+GmGmUmC+GmCmCmA+TmGmGmC |
| | GGCGG | +GmG |
| 264 | GCCAGGUAAGUAUU | mGmCmCmAmGmGmUmAmAmGmUmAmUmUmU+C |
| | UCCAGGGUCGCCAT | mCmAmG+GmGmUmC+GmCmCmA+TmG*mG*mC*+ |
| | GGCGG | G*mG |
| 265 | GCCAGGUAAGUAUU | mG*mC*mC*mA*mGmGmUmAmAmGmUmAmUmUm |
| | UCCAGGGUCGCCAT | U+CmCmAmG+GmGmUmC+GmCmCmA+TmG*mG*m |
| | GGCGG | C*+G*mG |
| 266 | GCCAGGUAAGUAUU | mGmCmCmAmGmGmUmAmAmGmUmAmU/i2MOErT |
| | UCCAGGGUCGCCAT | //i2MOErT/+C/i2MOErC//i2MOErA//i2MOErG/+G/ |
| | GGCGG | i2MOErG//i2MOErT//i2MOErC/+G/i2MOErC//i2MOErC// |
| | | i2MOErA/+T/i2MOErG/*/i2MOErG/*/i2MOErC/*+G*/ |
| | | 32MOErG/ |
| 267 | GCCAGGUAAGUAUU | mG*mC*mC*mA*mGmGmUmAmAmGmUmAmU/ |
| | UCCAGGGUCGCCAT | i2MOErT//i2MOET/+C/i2MOErC//i2MOErA//i2MOErG/+G/ |
| | GGCGG | i2MOErG//i2MOErT//i2MOErC/+G/i2MOErC//i2MOErC/ |
| | | /i2MOErA/+T/i2MOErG/*/i2MOErG/*/i2MOErC/*+G*/ |
| | | 32MOErG/ |
| 268 | GCCAGGUAAGUAUU | /52MOErG/*+C*/i2MOErC/*+A*/i2MOErG/+G/ |
| | UCCAGGGUCGCCAT | i2MOErT/+A/i2MOErA/+G/i2MOErT//i2MOErA//i2MOErT/ |
| | GGCGG | mUmU+CmCmAmG+GmGmUmC+GmCmCmA+TmGmGm |
| | | C+GmG |
| 269 | GCCAGGUAAGUAUU | /52MOErG/*+C*/i2MOErC/*+A*/i2MOErG/+G/ |
| | UCCAGGGUCGCCAT | i2MOErT/+A/i2MOErA/+G/i2MOErT//i2MOErA//i2MOErT/ |
| | GGCGG | mUmU+CmCmAmG+GmGmUmC+GmCmCmA+TmG*mG* |
| | | mC*+G*mG |
| 270 | GCCAGGUAAGUAUU | /52MOErG/*+C*/i2MOErC/*+A*/i2MOErG/+G/ |
| | UCCAGGGUCGCCAT | i2MOErT/+A/i2MOErA/+G/i2MOErT//i2MOErA//i2MOErT// |
| | GGCGG | i2MOErT//i2MOErT/+C/i2MOErC//i2MOErA//i2MOErG/+G/ |
| | | i2MOErG//i2MOErT//i2MOErC/+G/i2MOErC//i2MOErC/ |
| | | /i2MOErA/+T/i2MOErG/*/i2MOErG/*/i2MOErC/*+G*/ |
| | | 32MOErG/ |
| 271 | GCCAGGUAAGUAUU | mG*mC*mC*mA*mGmGmUmAmAmGmUmAmUmUm |
| | CAGCUTUUCCAGGG | C+AmGmCmU+TmUmUmC+CmAmGmG+GmUmCmG |
| | UCGCC | +CmC |
| 272 | GCCAGGUAAGUAUU | mGmCmCmAmGmGmUmAmAmGmUmAmUmUmC+A |
| | CAGCUTUUCCAGGG | mGmCmU+TmUmUmC+CmAmGmG+GmU*mC*mG*+ |
| | UCGCC | C*mC |
| 273 | GCCAGGUAAGUAUU | mG*mC*mC*mA*mGmGmUmAmAmGmUmAmUmUm |
| | CAGCUTUUCCAGGG | C+AmGmCmU+TmUmUmC+CmAmGmG+GmU*mC*m |
| | UCGCC | G*+C*mC |
| 274 | GCCAGGUAAGUAUU | mGmCmCmAmGmGmUmAmAmGmUmAmU/i2MOErT |
| | CAGCUTUUCCAGGG | //i2MOErC/+A/i2MOErG//i2MOErC//i2MOErT/+T/ |
| | UCGCC | i2MOErT//i2MOErT//i2MOErC/+C/i2MOErA//i2MOErG// |
| | | i2MOErG/+G/i2MOErT/*/i2MOErC/*/i2MOErG/*+C*/ |
| | | 32MOErC/ |
| 275 | GCCAGGUAAGUAUU | mG*mC*mC*mA*mGmGmUmAmAmGmUmAmU/ |
| | CAGCUTUUCCAGGG | i2MOErT//i2MOErC/+A/i2MOErG//i2MOErC//i2MOErT/+T/ |
| | UCGCC | i2MOErT//i2MOErT//i2MOErC/+C/i2MOErA//i2MOErG// |
| | | i2MOErG/+G/i2MOErT/*/i2MOErC/*/i2MOErG/*+C*/ |
| | | 32MOErC/ |
| 276 | GCCAGGUAAGUAUU | /52MOErG/*+C*/i2MOErC/*+A*/i2MOErG/+G/ |
| | CAGCUTUUCCAGGG | i2MOErT/+A/i2MOErA/+G/i2MOErT//i2MOErA//i2MOErT/ |
| | UCGCC | mUmC+AmGmCmU+TmUmUmC+CmAmGmG+GmUmCm |
| | | G+CmC |
| 277 | GCCAGGUAAGUAUU | /52MOErG/*+C*/i2MOErC/*+A*/i2MOErG/+G/ |
| | CAGCUTUUCCAGGG | i2MOErT/+A/i2MOErA/+G/i2MOErT//i2MOErA//i2MOErT/ |
| | UCGCC | mUmC+AmGmCmU+TmUmUmC+CmAmGmG+GmU*mC* |
| | | mG*+C*mC |
| 278 | GCCAGGUAAGUAUU | /52MOErG/*+C*/i2MOErC/*+A*/i2MOErG/+G/ |
| | CAGCUTUUCCAGGG | i2MOErT/+A/i2MOErA/+G/i2MOErT//i2MOErA//i2MOErT// |
| | UCGCC | i2MOErT//i2MOErC/+A/i2MOErG//i2MOErC//i2MOErT/+T/ |
| | | i2MOErT//i2MOErT//i2MOErC/+C/i2MOErA//i2MOErG// |
| | | i2MOErG/+G/i2MOErT/*/i2MOErC/*/i2MOErG/*+C*/ |
| | | 32MOErC/ |
| 279 | GCCAGGUAAGUAUA | mG*mC*mC*mA*mGmGmUmAmAmGmUmAmUmAm |
| | AGGACTUGAGGGAC | A+GmGmAmC+TmUmGmA+GmGmGmA+CmUmCmG |
| | UCGAA | +AmA |
| 280 | GCCAGGUAAGUAUA | mGmCmCmAmGmGmUmAmAmGmUmAmUmAmA+G |
| | AGGACTUGAGGGAC | mGmAmC+TmUmGmA+GmGmGmA+CmU*mC*mG*+ |
| | UCGAA | A*mA |
| 281 | GCCAGGUAAGUAUA | mG*mC*mC*mA*mGmGmUmAmAmGmUmAmUmAm |
| | AGGACTUGAGGGAC | A+GmGmAmC+TmUmGmA+GmGmGmA+CmU*mC* |
| | UCGAA | mG*+A*mA |
| 282 | GCCAGGUAAGUAUA | mGmCmCmAmGmGmUmAmAmGmUmAmU/i2MOErA |
| | AGGACTUGAGGGAC | //i2MOErA/+G/i2MOErG//i2MOErA//i2MOErC/+T/ |
| | UCGAA | i2MOErT//i2MOErG//i2MOErA/+G/i2MOErG//i2MOErG// |
| | | i2MOErA/+C/i2MOErT/*/i2MOErC/*/i2MOErG/*+A*/ |
| | | 32MOErA/ |
| 283 | GCCAGGUAAGUAUA | mG*mC*mC*mA*mGmGmUmAmAmGmUmAmU/ |
| | AGGACTUGAGGGAC | i2MOErA//i2MOErA/+G/i2MOErG//i2MOErA//i2MOErC/+T/ |
| | UCGAA | i2MOErT//i2MOErG//i2MOErA/+G/i2MOErG//i2MOErG |
| | | //i2MOErA/+C/i2MOErT/*/i2MOErC/*/i2MOErG/*+A*/ |
| | | 32MOErA/ |
| 284 | GCCAGGUAAGUAUA | /52MOErG/*+C*/i2MOErC/*+A*/i2MOErG/+G/ |
| | AGGACTUGAGGGAC | i2MOErT/+A/i2MOErA/+G/i2MOErT//i2MOErA//i2MOErT/ |
| | UCGAA | mAmA+GmGmAmC+TmUmGmA+GmGmGmA+CmUmCm |
| | | G+AmA |
| 285 | GCCAGGUAAGUAUA | /52MOErG/*+C*/i2MOErC/*+A*/i2MOErG/+G/ |
| | AGGACTUGAGGGAC | i2MOErT/+A/i2MOErA/+G/i2MOErT//i2MOErA//i2MOErT/ |
| | UCGAA | mAmA+GmGmAmC+TmUmGmA+GmGmGmA+CmU*mC |
| | | *mG*+A*mA |
| 286 | GCCAGGUAAGUAUA | /52MOErG/*+C*/i2MOErC/*+A*/i2MOErG/+G/ |
| | AGGACTUGAGGGAC | i2MOErT/+A/i2MOErA/+G/i2MOErT//i2MOErA//i2MOErT// |
| | UCGAA | i2MOErA//i2MOErA/+G/i2MOErG//i2MOErA//i2MOErC/+T/ |
| | | i2MOErT//i2MOErG//i2MOErA/+G/i2MOErG//i2MOErG |
| | | //i2MOErA/+C/i2MOErT/*/i2MOErC/*/i2MOErG/*+A*/ |
| | | 32MOErA/ |
| 287 | GCCAGGUAAGUAUU | mG*mC*mC*mA*mGmGmUmAmAmGmUmAmUmUm |
| | UCAUCAGCUTUUCC | U+CmAmUmC+AmGmCmU+TmUmUmC+CmAmGmG |
| | AGGGU | +GmU |
| 288 | GCCAGGUAAGUAUU | mGmCmCmAmGmGmUmAmAmGmUmAmUmUmU+C |
| | UCAUCAGCUTUUCC | mAmUmC+AmGmCmU+TmUmUmC+CmA*mG*mG*+ |
| | AGGGU | G*mU |
| 289 | GCCAGGUAAGUAUU | mG*mC*mC*mA*mGmGmUmAmAmGmUmAmUmUm |
| | UCAUCAGCUTUUCC | U+CmAmUmC+AmGmCmU+TmUmUmC+CmA*mG*m |
| | AGGGU | G*+G*mU |
| 290 | GCCAGGUAAGUAUU | mGmCmCmAmGmGmUmAmAmGmUmAmU/i2MOErT |
| | UCAUCAGCUTUUCC | //i2MOErT/+C/i2MOErA//i2MOErT//i2MOErC/+A/ |
| | AGGGU | i2MOErG//i2MOErC//i2MOErT/+T/i2MOErT//i2MOErT// |
| | | i2MOErC/+C/i2MOErA/*/i2MOErG/*/i2MOErG/*+G*/ |
| | | 32MOErT/ |
| 291 | GCCAGGUAAGUAUU | mG*mC*mC*mA*mGmGmUmAmAmGmUmAmU/ |
| | UCAUCAGCUTUUCC | i2MOErT//i2MOErT/+C/i2MOErA//i2MOErT//i2MOErC/+A/ |
| | AGGGU | i2MOErG//i2MOErC//i2MOErT/+T/i2MOErT//i2MOErT// |
| | | i2MOErC/+C/i2MOErA/*/i2MOErG/*/i2MOErG/*+G*/ |
| | | 32MOErT/ |
| 292 | GCCAGGUAAGUAUU | /52MOErG/*+C*/i2MOErC/*+A*/i2MOErG/+G/ |
| | UCAUCAGCUTUUCC | i2MOErT/+A/i2MOErA/+G/i2MOErT//i2MOErA//i2MOErT/ |
| | AGGGU | mUmU+CmAmUmC+AmGmCmU+TmUmUmC+CmAmGm |
| | | G+GmU |
| 293 | GCCAGGUAAGUAUU | /52MOErG/*+C*/i2MOErC/*+A*/i2MOErG/+G/ |
| | UCAUCAGCUTUUCC | i2MOErT/+A/i2MOErA/+G/i2MOErT//i2MOErA//i2MOErT/ |
| | AGGGU | mU+CmAmUmC+AmGmCmU+TmUmUmC+CmA*mG* |
| | | mUmG*+G*mU |
| 294 | GCCAGGUAAGUAUU | /52MOErG/*+C*/i2MOErC/*+A*/i2MOErG/+G/ |
| | UCAUCAGCUTUUCC | i2MOErT/+A/i2MOErA/+G/i2MOErT//i2MOErA//i2MOErT// |
| | AGGGU | i2MOErT//i2MOErT/+C/i2MOErA//i2MOErT//i2MOErC/+A/ |
| | | i2MOErG//i2MOErC//i2MOErT/+T/i2MOErT//i2MOErT// |
| | | i2MOErC/+C/i2MOErA/*/i2MOErG/*/i2MOErG/*+G*/ |
| | | 32MOErT/ |

The bifunctional oligonucleotide may comprise nucleotide sequence with at least 75%, 80%, 85%, 90%, 95%, 99%, or more sequence identity with a nucleotide selected from SEQ ID NOs: 100-294, or a variant or fragment thereof. In an embodiment, the bifunctional oligonucleotide comprises nucleotide sequence selected from SEQ ID NOs: 100-294, or a variant or fragment thereof. In an embodiment, the bifunctional oligonucleotide comprises a nucleotide sequence with at least 75%, 80%, 85%, 90%, 95%, 99%, or more sequence identity SEQ ID NO: 004. In an embodiment, the bifunctional oligonucleotide comprises a spliceosome targeting sequence comprising SEQ ID NO: 004.

The bifunctional oligonucleotide may comprise nucleotide sequence with at least 75%, 80%, 85%, 90%, 95%, 99%, or more sequence identity with a nucleotide selected from SEQ ID NOs: 100-254, or a variant or fragment thereof. In an embodiment, the bifunctional oligonucleotide comprises nucleotide sequence selected from SEQ ID NOs: 100-254, or a variant or fragment thereof. In an embodiment, the bifunctional oligonucleotide comprises a nucleotide sequence with at least 75%, 80%, 85%, 90%, 95%, 99%, or more sequence identity SEQ ID NO: 004. In an embodiment, the bifunctional oligonucleotide comprises a spliceosome targeting sequence comprising SEQ ID NO: 004.

In an embodiment of Formulas (I), (I-a), (I-b), (I-c), (I-d), (I-e), and (I-f), bases 1-15, 17-19, 21-23, 25-27, 29-31, and 33 comprise a 2′OMe modification; bases 2, 3, 4, and 5 comprise a phosphorothioate modification; and bases 16, 20, 24, 28, and 32 comprise an LNA modification.

In an embodiment of Formulas (I), (I-a), (I-b), (I-c), (I-d), (I-e), and (I-f), bases 1-15, 17-19, 21-23, 25-27, 29-31, and 33 comprise a 2′OMe modification; bases 30, 31, 32, and 33 comprise a phosphorothioate modification; and bases 16, 20, 24, 28, and 32 comprise an LNA modification.

In an embodiment of Formulas (I), (I-a), (I-b), (I-c), (I-d), (I-e), and (I-f), bases 1-15, 17-19, 21-23, 25-27, 29-31, and 33 comprise a 2′OMe modification; bases 2, 3, 4, 5, 30, 31, 32, and 33 comprise a phosphorothioate modification; and bases 16, 20, 24, 28, and 32 comprise an LNA modification.

In an embodiment of Formulas (I), (I-a), (I-b), (I-c), (I-d), (I-e), and (I-f), bases 1-13 comprise a 2′OMe modification; bases 14, 15, 17-19, 21-23, 25-27, 29-31, and 33 comprise a 2′-O-methoxy ethyl modification; bases 30, 31, 32, and 33 comprise a phosphorothioate modification; and bases 16, 20, 24, 28, and 32 comprise an LNA modification.

In an embodiment of Formulas (I), (I-a), (I-b), (I-c), (I-d), (I-e), and (I-f), bases 1-13 comprise a 2′OMe modification; bases 14, 15, 17-19, 21-23, 25-27, 29-31, and 33 comprise a 2′-O-methoxy ethyl modification; bases 2, 3, 4, 5, 30, 31, 32, and 33 comprise a phosphorothioate modification; and bases 16, 20, 24, 28, and 32 comprise an LNA modification.

In an embodiment of Formulas (I), (I-a), (I-b), (I-c), (I-d), (I-e), and (I-f), bases 1, 3, 5, 7, 9, and 11-13 comprise 2′-O-methoxy ethyl modification; bases 14, 15, 17-19, 21-23, 25-27, 29-31, and 33 comprise a 2′OMe modification; bases 2, 3, 4, 5, 30, 31, 32, and 33 comprise a phosphorothioate modification; and bases 2, 4, 6, 8, 10, 16, 20, 24, 28, and 32 comprise an LNA modification.

In an embodiment of Formulas (I), (I-a), (I-b), (I-c), (I-d), (I-e), and (I-f), bases 1, 3, 5, 7, 9, 11-13, 14, 15, 17-19, 21-23, 25-27, 29-31, and 33 comprise a 2′-O-methoxy ethyl modification; bases 2, 3, 4, 5, 30, 31, 32, and 33 comprise a phosphorothioate modification; and bases 2, 4, 6, 8, 10, 16, 20, 24, 28, and 32 comprise an LNA modification.

In an embodiment of Formulas (I), (I-a), (I-b), (I-c), (I-d), (I-e), and (I-f), bases 1, 3, 5, 7, 9, and 11-13 comprise 2′-O-methoxy ethyl modification; bases 14, 15, 17-19, 21-23, 25-27, 29-31, and 33 comprise a 2′OMe modification; bases 2, 3, 4, and 5 comprise a phosphorothioate modification; and bases 2, 4, 6, 8, 10, 16, 20, 24, 28, and 32 comprise an LNA modification.

In an embodiment, the bifunctional oligonucleotide comprises a chemical modification that extends the half-life of the bifunctional oligonucleotide in serum, plasma, a cell, or a subject.

In an embodiment, the bifunctional oligonucleotide has a half-life in serum plasma, a cell, or a subject of at least about 2 hours, e.g., at least about 3 hours, at least about 4 hours, at least about 5 hours, at least about 6 hours, at least about 8 hours, at least about 10 hours, or at least about 12 hours. In some embodiments, the nucleic acid agent modified as described herein has a half-life in liver homogenate (e.g., rat serum) of at least about 2 hours, e.g., at least about 3 hours, at least about 4 hours, at least about 5 hours, at least about 6 hours, at least about 8 hours, at least about 10 hours, at least about 12 hours, or at least about 24 hours

In an embodiment, the bifunctional oligonucleotide comprises (i) a nucleotide sequence capable of binding to a target sequence comprising an exonic element, e.g., an alternative splice site (e.g., 5′ splice site) within exon 1 of the HTT gene, wherein the nucleotide sequence comprises a plurality of chemical modifications (e.g., a plurality of 2′OMe modifications and LNA modifications) and is between 5 and 35 nucleotides in length; and (b) a nucleotide sequence capable of binding an U1 snRNA comprising a plurality of chemical modifications (e.g., a plurality of 2′OMe modifications).

In an embodiment, the bifunctional oligonucleotide is capable of one or more of (a) enhancing exonization of the HTT gene (e.g., exon 1) by recruiting the U1 snRNP to a non-canonical 5′ splice site; and (b) potentiating U1 usage/recruitment. In an embodiment, the bifunctional oligonucleotide is capable of (a). In an embodiment, the bifunctional oligonucleotide is capable of (b).

The compounds (e.g., the bifunctional oligonucleotides) provided herein may contain one or more asymmetric centers and thus occur as racemates and racemic mixtures, single enantiomers, individual diastereomers and diastereomeric mixtures. All such isomeric forms of these compounds are expressly included within the scope. Unless otherwise indicated when a compound is named or depicted by a structure without specifying the stereochemistry and has one or more chiral centers, it is understood to represent all possible stereoisomers of the compound. The compounds provided herewith may also contain linkages (e.g., carbon-carbon bonds, phosphorus-oxygen bonds, or phosphorus-sulfur bonds) or substituents that can restrict bond rotation, e.g. restriction resulting from the presence of a ring or double bond.

Without wishing to be bound by theory, the mechanism of action of a bifunctional oligonucleotide may proceed in a number of ways. For example, the bifunctional oligonucleotide may first bind through its alternative splice site targeting sequence to an upstream alternative splice site within a target gene comprising multiple trinucleotide repeats, and then recruit the U1 splicing machinery to initiate splicing. It is hypothesized that in certain cases, as the trinucleotide repeat expansion sequence increases in size (e.g., as in HTT CAG repeats), the repeat expansion becomes inhibitory for the canonical 5′ splice site and may result in an exon 1 fragment due to unproductive splicing. As such, using the bifunctional oligonucleotides to recruit the U1 splicing machinery to a novel site, e.g., upstream to the canonical 5′ splice site, may enhance exonization at this location and, e.g., result in skipping over the inhibitory CAG repeats.

### Methods of Making Bifunctional Oligonucleotides

The bifunctional oligonucleotides described herein can be prepared using solution-phase or solid-phase organic synthesis. Organic synthesis offers the advantage that the oligonucleotide strands comprising non-natural or modified nucleotides can be easily prepared. Any other means for such synthesis known in the art may additionally or alternatively be employed. It is also known to use similar techniques to prepare other bifunctional oligonucleotides, such as the phosphorothioates, phosphorodithioates and alkylated derivatives. Regardless of the method of synthesis, the bifunctional oligonucleotide can be prepared in a solution (e.g., an aqueous and/or organic solution) that is appropriate for formulation. For example, the bifunctional oligonucleotide preparation can be precipitated and redissolved in pure double-distilled water, and lyophilized. The dried bifunctional oligonucleotide can then be resuspended in a solution appropriate for the intended formulation process. Teachings regarding the synthesis of particular modified oligonucleotides may be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; each of which is incorporated herein by reference in its entirety.

In an embodiment, bifunctional oligonucleotides described herein are prepared by connecting nucleosides with optionally protected phosphorus containing internucleoside linkages. Representative protecting groups for phosphorus containing internucleoside linkages such as phosphodiester and phosphorothioate linkages include β-cyanoethyl, diphenylsilylethyl, 6-cyanobutenyl, cyano p-xylyl (CPX), N-methyl-N-trifluoroacetyl ethyl (META), acetoxy phenoxy ethyl (APE) and butene-4-yl groups. See for example U.S. Pat. Nos. 4,725,677 and Re. 34,069 (O-cyanoethyl); Beaucage, S. L. and Iyer, R. P., Tetrahedron, 49 No. 10, pp. 1925-1963 (1993); Beaucage, S. L. and Iyer, R. P., Tetrahedron, 49 No. 46, pp. 10441-10488 (1993); Beaucage, S. L. and Iyer, R. P., Tetrahedron, 48 No. 12, pp. 2223-2311 (1992).

In an embodiment, nucleosides having reactive phosphorus groups are provided that are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. A preferred synthetic solid phase synthesis utilizes phosphoramidites (P^{III} chemistry) as reactive phosphites. The intermediate phosphite compounds are subsequently oxidized to the Pv state using known methods to yield, in preferred embodiments, phosphodiester or phosphorothioate internucleotide linkages.

### Methods of Delivery

A bifunctional oligonucleotide described herein may comprise, may be formulated with, or may be delivered in, a carrier. For example, the bifunctional oligonucleotide may be disposed in a vesicle or other membrane-based carrier, such as a liposome or lipid nanoparticle.

In one embodiment, the bifunctional oligonucleotides described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review).

Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the bifunctional oligonucleotides described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exemplary lipid nanoparticles are disclosed in International Application PCT/US2014/053907, the entire contents of which are hereby incorporated by reference. For example, an LNP described in paragraphs [403-406] or [410-413] of PCT/US2014/053907 can be used as a carrier for the bifunctional oligonucleotide described herein.

Additional exemplary lipid nanoparticles are disclosed in U.S. Pat. No. 10,562,849 the entire contents of which are hereby incorporated by reference. For example, an LNP of formula (I) as described in columns 1-3 of U.S. Pat. No. 10,562,849 can be used as a carrier for the bifunctional oligonucleotides described herein.

Lipids that can be used in nanoparticle formations (e.g., lipid nanoparticles) include, for example those described in Table 4 of WO2019217941, which is incorporated by reference, e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL In some embodiments, a composition described herein (e.g., bifunctional oligonucleotide composition) is provided in an LNP that comprises an ionizable lipid. In some embodiments, the ionizable lipid is heptadecan-9-yl 8-((2-hydroxyethyl)(6-oxo-6-(undecyloxy)hexyl)-amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LPO1), e.g., as synthesized in Example 13 of WO2015/095340 (incorporated by reference herein in its entirety). In some embodiments, the ionizable lipid is Di((Z)-non-2-en-1-yl) 9-((4-dimethylamino)-butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a bifunctional oligonucleotide described herein, encapsulated within or associated with the lipid nanoparticle. In some embodiments, the bifunctional oligonucleotide is co-formulated with the cationic lipid. The bifunctional oligonucleotide may be adsorbed to the surface of an LNP, e.g., an LNP comprising a cationic lipid. In some embodiments, the bifunctional oligonucleotide may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of a bifunctional oligonucleotide.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, 11, III, or IV of WO2009/132131; A of US2012/01011478; I or XXXV of US2012/0027796; XIV or XVII of US2012/0058144; of US2013/0323269; I of US2011/0117125; 1, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; 1-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 1Z)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, l-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.Oc01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2′-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-l-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000].

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a bifunctional oligonucleotide describes the amount of bifunctional oligonucleotide that is encapsulated or otherwise associated with a LNP after preparation, relative to the initial amount provided. The encapsulation efficiency is desirably high (e.g., close to 100%). The encapsulation efficiency may be measured, for example, by comparing the amount of bifunctional oligonucleotide in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free bifunctional oligonucleotide in a solution. For the lipid nanoparticles described herein, the encapsulation efficiency of a bifunctional oligonucleotide may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Delivery without a Carrier

A bifunctional oligonucleotide described herein can be administered to a cell without a carrier, e.g., via naked delivery of the bifunctional oligonucleotide. In some embodiments, naked delivery as used herein refers to delivery without a carrier. In some embodiments, delivery without a carrier, e.g., naked delivery, comprises delivery with a moiety, e.g., a targeting peptide.

In some embodiments, a bifunctional oligonucleotide described herein is delivered to a cell without a carrier, e.g., via naked delivery. In some embodiments, the delivery without a carrier, e.g., naked delivery, comprises delivery with a moiety, e.g., a targeting peptide.

### Methods of Use

Described herein are bifunctional compounds useful for modulating splicing at a mutant splice site, e.g., in a gene or transcript comprising a trinucleotide repeat expansion. In some embodiments, a bifunctional oligonucleotide described herein may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a bifunctional oligonucleotide described herein may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a bifunctional oligonucleotide described herein. In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a bifunctional oligonucleotide described herein. In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35% 40%, 45%, 50% 55%, 60%, 65%, 70%, 75%, 80%, 85%, 90%, 95%, 99%, or more), e.g., as compared to a reference (e.g., the absence of a bifunctional oligonucleotide described herein, or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, AFF3, AR, ARX, ATN1, ATXN1, ATXN2, ATXN3, ATXN7, ATXN8OS, ATXN8b, CBL2, COMP, DMPK, FMR1, FOXL2, GIPC1, GLS, HOXD13, HTT, JPH3, LOC642361, NUTM2b-AS1, PHOX2B, RUNX2, SOX3, TBP, and ZIC2. In an embodiment, the target gene is AFF3. In an embodiment, the target gene is AR. In an embodiment, the target gene is ARX. In an embodiment, the target gene is ATN1. In an embodiment, the target gene is ATXN1. In an embodiment, the target gene is ATXN2. In an embodiment, the target gene is ATXN3. In an embodiment, the target gene is ATXN7. In an embodiment, the target gene is ATXN8OS. In an embodiment, the target gene is ATXN8b. In an embodiment, the target gene is CBL2. In an embodiment, the target gene is COMP. In an embodiment, the target gene is DMPK. In an embodiment, the target gene is FMR1. In an embodiment, the target gene is FOXL2. In an embodiment, the target gene is GIPC1. In an embodiment, the target gene is GLS. In an embodiment, the target gene is HOXD13. In an embodiment, the target gene is HTT. In an embodiment, the target gene is JPH3. In an embodiment, the target gene is LOC642361. In an embodiment, the target gene is NUTM2b-AS1. In an embodiment, the target gene is PHOX2B. In an embodiment, the target gene is RUNX2. In an embodiment, the target gene is SOX3. In an embodiment, the target gene is TBP. In an embodiment, the target gene is ZIC2.

In another aspect, the present disclosure features methods for modulating the production of a transcription product in a subject having a neurological disease or disorder. In an embodiment, the neurological disease or disorder is a repeat expansion disease (e.g., a trinucleotide repeat expansion disease). Exemplary diseases and disorders include Huntington's disease, Huntington's disease-like 2, holoprosencephaly 5, spinocerebellar ataxia type 1, spinocerebellar ataxia type 2, spinocerebellar ataxia type 3, spinocerebellar ataxia type 7, spinocerebellar ataxia type 8, spinocerebellar ataxia type 17, myotonic dystrophy type 1, oculopharyngodistal myopathy 2, oculopharyngodistal myopathy with leukoencephalopathy, X-linked intellectual disability, dentatorubral-paltidoluysian atrophy, spinal and bulbar atrphy, cleidocranial dysplasia, synpolydactyly 1, glutaminase deficiency, Jacobsen syndrome, fragile X syndrome, fragile X-associated primary ovarian insufficiency, fragile X-associated tremor/ataxia syndrome, X-linked hypopituitarism, and congenital central hypoventilation syndrome.

In an embodiment, the trinucleotide repeat comprises CXY, where X and Y are each selected from any one of A, T, C, and G. In an embodiment, the trinucleotide repeat comprises CAG, CTG, CGG, CCG, or GCN. In an embodiment, the target gene, trinucleotide repeat, and number of trinucleotide repeats is selected from one in Table 4.

TABLE 4
Exemplary repeat expansion gene mutations

| | | Repeat | | | Number |
|---|---|---|---|---|---|
| Number | Host Gene | Motif | Location | Repeat Location | of Repeats |
| 1 | HTT | CAG | CDS | chr4: 3074886-3074936: + | 16 |
| 2 | ATXN1 | CAG | CDS | chr6: 16327640-16327710: − | 14 |
| 3 | ATXN3 | CAG | CDS | chr14: 92071003-92071053: − | 11 |
| 4 | DMPK | CTG | 3′ UTR | chr19: 45770210-45770260: − | 16 |
| 5 | GIPC1 | CGG | 5′ UTR | chr19: 14496039-14496089: − | 12 |
| 6 | ARX | GCN | CDS | chrX: 25013648-25013698: − | 16 |
| 7 | ATN1 | CAG | CDS | chr12: 6936727-6936777: + | 15 |
| 8 | AR | CAG | CDS | chrX: 67545327-67545377: + | 16 |
| 9 | ATXN7 | CAG | CDS | chr3: 63912679-63912729: + | 10 |
| 10 | TBP | CAG | CDS | chr6: 170561951-170562021: + | 15 |
| 11 | COMP | GAC | CDS | chr19: 18785996-18786066: − | 7 |
| 12 | RUNX2 | GCN | CDS | chr6: 45422757-45422807: + | 16 |
| 13 | HOXD13 | GCN | CDS | chr2: 176093051-176093101: + | 15 |
| 14 | ATXN2 | CAG | CDS | chr12: 111598964-111599014: − | 15 |
| 15 | GLS | CAG | 5′ UTR | chr2: 190880867-190880917: + | 14 |
| 16 | CBL2 | CCG | 5′ UTR | chr11: 119206376-119206426: + | 4 |
| 17 | AFF3 | CGG | 5′ UTR | chr2: 100104468-100104518: − | 9 |
| 18 | FMR1 | CGG | 5′ UTR | chrX: 147912051-147912101: + | 15 |
| 19 | FMR1 | CGG | 5′ UTR | chrX: 147912051-147912101: + | 15 |
| 20 | FMR1 | CGG | 5′ UTR | chrX: 147912051-147912101: + | 15 |
| 21 | LOC642361 and | CGG/CCG | lncRNA | chr10: 79826364-79826414: − | 10 |
| | NUTM2b-AS1 | | | | |
| 22 | ATXN8OS and | CTG/CAG | 3′ UTR | chr13: 70139382-70139430: + | — |
| | ATXN8b | | and CDS | | |
| 23 | JPH3 | CTG | 3′ UTR | chr16: 87697181-87697231: + | 5 |
| 24 | FOXL2 | GCN | CDS | chr3: 138946012-138946062: − | 15 |
| 25 | PHOX2B | GCN | CDS | chr4: 41745972-41746022: − | 16 |
| 26 | ZIC2 | GCN | CDS | chr13: 99985443-99985493: + | 14 |
| 27 | SOX3 | GCN | CDS | chrX: 140504310-140504360: − | 15 |
| 28 | LOC642361 and | CGG/CCG | lncRNA | chr10: 79826362-79826412: + | 10 |
| | NUTM2b-AS1 | | | | |

### Pharmaceutical Compositions, Kits, and Administration

The present invention provides pharmaceutical compositions comprising a bifunctional oligonucleotide, e.g., a bifunctional oligonucleotide or a pharmaceutically acceptable salt, solvate, hydrate, tautomer, or stereoisomer, as described herein, and optionally a pharmaceutically acceptable excipient. In certain embodiments, the pharmaceutical composition described herein comprises a bifunctional oligonucleotide or a pharmaceutically acceptable salt thereof, and optionally a pharmaceutically acceptable excipient. In certain embodiments, the bifunctional oligonucleotide or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the bifunctional oligonucleotide (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The bi or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the bifunctional oligonucleotide or composition described herein in a single dose or administered separately in different doses. The particular combination to employ in a regimen will take into account compatibility of the inventive bifunctional oligonucleotide with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug bifunctional oligonucleotides (e.g., bifunctional oligonucleotides approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or bifunctional oligonucleotide and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or bifunctional oligonucleotide. In some embodiments, the inventive pharmaceutical composition or bifunctional oligonucleotide provided in the container and the second container are combined to form one-unit dosage form.

Thus, in one aspect, provided are kits including a first container comprising a bifunctional oligonucleotide described herein, or a pharmaceutically acceptable salt, solvate, hydrate, tautomer, or stereoisomer thereof, or a pharmaceutical composition thereof. In certain embodiments, the kit of the disclosure includes a first container comprising a bifunctional oligonucleotide described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the bifunctional oligonucleotide, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### EXAMPLES

In order that the disclosure described herein may be more fully understood, the following examples are set forth. The examples described in this application are offered to illustrate the bifunctional oligonucleotides, compositions, and methods provided herein and are not to be construed in any way as limiting their scope.

### Example 1: Design and Synthesis of Exemplary Bifunctional Oligonucleotides

Bifunctional oligonucleotides described herein were designed to contain at least two distinct targeting sequences, namely an alternate splice site targeting sequence and a spliceosome targeting sequence. The bifunctional oligonucleotides were synthesized from commercially available nucleotide building blocks using standard solid phase synthesis techniques. Exemplary sequences and the corresponding molecular weights are summarized in Table 1.

### Example 2: Transfection of Huntington (HTT) Minigenes and Exemplary Bifunctional Oligonucleotides into HEK2932T Cells

In order to screen exemplary bifunctional oligonucleotides for activity against HTT minigenes, a cell-based assay was developed in HEK2932T cells. Roughly 30,000 HEK293T cells were plated per well in a 96 well plate. Various HTT mingenes, either wild type or mutant, were transfected into the cells using Lipofectamine 3000 as a transfection agent. After 24 hours, the transfected cells were treated with varying concentrations of bifunctional oligos and control oligonucleotides, ranging from 0-100 nM, using Lipofectamine 2000 as a transfection agent. The cells were lysed between 48-72 hours after treatment with the bifunctional oligonucleotides using a lysis buffer (2% Igepal with 0.1 U/uL RNAsin), and the cell lysates were used directly for qPCR assays (described herein in Example 1) using HTT-minigene specific primer probe sets to detect a splicing event.

### Example 3: Transfection of Exemplary Bifunctional Oligonucleotides into Huntington Disease (HD) Patient-Derived Primary Fibroblasts

Exemplary bifunctional oligonucleotides were also transfected into HD patient-derived fibroblast cells. Primary HD fibroblasts were used in this experiment, with the wild type cells from the GM07492 cell line and the mutant cells from the GM04857 cell line. Roughly 10,000 patient fibroblasts were plated per well in a 96 well plate. After 24 hours, the cells were treated with varying concentrations of bifunctional oligos and control oligonucleotides, ranging from 0-100 nM, using Lipofectamine 2000 as a transfection agent. The cells were lysed between 48-72 hours after treatment with the bifunctional oligonucleotides using a lysis buffer (2% Igepal with 0.1 U/uL RNAsin), and the cell lysates were used directly for qPCR assays using endogenous HTT-specific primer sets. The results are shown in Table 5, where AJ<1.25, “−”, 1 0.25<=AJ<2.5, ‘Y’, AJ>=2.5, “++”, CJ>0.9, “−” 0.75<CJ<=0.9, “+”, CJ<=0.75, “++”.

TABLE 5

| | BFO (25 nM) | | | | BFO (100 nM) | | | |
|---|---|---|---|---|---|---|---|---|
| SEQ | | | | Primary | | | | Primary |
| ID NO. | AJ1 | AJ2 | CJ | hits | AJ1 | AJ2 | CJ | hits |
| 100 | − | + | − | | − | − | − | |
| 101 | − | + | − | | − | + | − | |
| 102 | + | − | − | | + | + | − | |
| 103 | − | − | − | | + | − | − | |
| 104 | − | − | − | | + | − | − | |
| 105 | − | − | − | | − | − | − | |
| 106 | + | + | − | | + | − | − | |
| 107 | + | − | − | | − | − | − | |
| 108 | + | + | − | | − | − | − | |
| 109 | − | + | − | | + | − | − | |
| 110 | ++ | ++ | − | | + | ++ | − | |
| 111 | ++ | − | − | | − | + | ++ | Y |
| 112 | − | − | − | | − | − | − | |
| 113 | − | − | − | | − | − | − | |
| 114 | − | − | − | | − | − | − | |
| 115 | + | + | − | | − | − | − | |
| 116 | − | − | − | | − | − | − | |
| 117 | + | − | − | | − | − | − | |
| 118 | ++ | − | − | | − | − | − | |
| 119 | − | − | − | | − | + | − | |
| 120 | + | + | − | | − | − | − | |
| 121 | + | − | − | | − | + | − | |
| 122 | + | − | ++ | Y | + | − | − | |
| 123 | ++ | − | − | | + | − | − | |
| 124 | + | − | − | | + | + | − | |
| 125 | + | − | − | | ++ | + | − | Y |
| 126 | − | + | − | | ++ | ++ | − | |
| 127 | − | − | − | | ++ | + | − | |
| 128 | + | − | − | | + | − | − | |
| 129 | − | + | − | | ++ | + | − | |
| 130 | + | + | − | | + | + | − | |
| 131 | + | − | − | | − | − | − | |
| 132 | − | − | − | | − | + | − | |
| 133 | − | + | − | | − | − | − | |
| 134 | + | − | − | | − | − | − | |
| 135 | + | − | + | Y | + | − | − | |
| 136 | − | − | − | | + | − | − | |
| 137 | + | − | − | | + | + | − | |
| 138 | − | + | − | | + | + | − | |
| 139 | + | − | − | | + | ++ | − | |
| 140 | − | − | − | | − | + | − | |
| 141 | − | − | − | | + | + | − | |
| 142 | ++ | − | − | | + | − | − | |
| 143 | + | + | − | | + | ++ | − | |
| 144 | − | − | ++ | | − | − | − | |
| 145 | − | − | + | | − | + | − | |
| 146 | + | − | ++ | Y | + | − | ++ | Y |
| 147 | − | − | ++ | | − | − | ++ | |
| 148 | − | − | − | | − | − | − | |
| 149 | + | − | − | | + | + | − | |
| 150 | − | − | − | | − | − | − | |
| 151 | − | − | − | | + | − | − | |
| 152 | − | − | ++ | | + | − | − | |
| 153 | − | − | − | | − | − | − | |
| 154 | + | + | − | | − | − | − | |
| 155 | − | − | + | | − | − | − | |
| 156 | − | − | + | | − | − | − | |
| 157 | − | + | − | | ++ | − | − | |
| 158 | − | − | − | | − | − | − | |
| 159 | ++ | − | − | | − | − | − | |
| 160 | − | − | − | | ++ | − | − | |
| 161 | + | + | − | | − | + | − | |
| 162 | − | − | − | | + | − | − | |
| 163 | − | − | − | | ++ | ++ | − | |
| 164 | + | − | − | | + | + | − | |
| 165 | + | − | − | | − | + | − | |
| 166 | − | + | − | | + | − | − | |
| 167 | − | − | − | | − | + | − | |
| 168 | − | − | − | | − | + | − | |
| 169 | − | − | − | | − | + | − | |
| 170 | − | − | − | | − | − | − | |
| 171 | − | − | − | | − | − | − | |
| 172 | − | − | − | | − | + | − | |
| 173 | − | − | − | | − | ++ | − | |
| 174 | − | − | − | | − | − | − | |
| 175 | − | − | − | | − | + | − | |
| 176 | − | + | − | | − | − | − | |
| 177 | + | − | − | | − | − | − | |
| 178 | + | + | − | | − | − | − | |
| 179 | + | + | − | | − | − | − | |
| 180 | + | − | − | | + | − | − | |
| 181 | − | − | + | | + | − | − | |
| 182 | ++ | − | − | | + | − | − | |
| 183 | + | − | − | | − | − | − | |
| 184 | ++ | − | − | Y | + | − | − | |
| 185 | + | + | − | | + | + | − | |
| 186 | + | − | − | | − | + | − | |
| 187 | − | − | + | | − | − | − | |
| 188 | + | ++ | + | Y | + | − | − | |
| 189 | + | + | − | | + | + | − | |
| 190 | + | ++ | − | | ++ | + | − | |
| 191 | ++ | ++ | − | Y | ++ | + | − | Y |
| 192 | + | + | − | | + | + | − | |
| 193 | + | − | − | | ++ | + | − | |
| 194 | ++ | ++ | − | | ++ | ++ | − | |
| 195 | ++ | ++ | ++ | Y | ++ | ++ | − | |
| 196 | + | − | − | | − | + | + | Y |
| 197 | ++ | − | ++ | Y | − | − | ++ | |
| 198 | ++ | + | − | | + | − | − | |
| 199 | − | + | − | | − | ++ | − | |
| 200 | − | − | − | | − | + | − | |
| 201 | − | + | − | | − | − | − | |
| 202 | − | − | − | | − | − | − | |
| 203 | − | − | − | | − | − | − | |
| 204 | − | + | − | | ++ | + | ++ | Y |
| 205 | − | − | − | | − | − | ++ | |
| 206 | + | − | ++ | Y | + | − | ++ | Y |
| 207 | − | − | − | | − | + | − | |
| 208 | − | − | − | | − | + | ++ | Y |
| 209 | − | − | + | | − | − | + | |
| 210 | − | − | ++ | | − | − | ++ | |
| 211 | + | + | − | | − | − | + | |
| 212 | − | − | + | | − | − | ++ | |
| 213 | − | − | − | | − | ++ | − | |
| 214 | − | − | ++ | | − | − | − | |
| 215 | − | + | − | | − | ++ | − | |
| 216 | − | − | ++ | | − | − | − | |
| 217 | − | − | ++ | | − | − | + | |
| 218 | − | − | ++ | Y | − | − | ++ | Y |
| 219 | + | − | ++ | Y | − | − | ++ | |
| 220 | − | − | ++ | | − | − | + | |
| 221 | + | − | + | Y | − | − | − | |
| 222 | − | − | − | | − | − | ++ | |
| 223 | − | − | − | | − | − | − | |
| 224 | − | − | ++ | | − | − | − | |
| 225 | − | − | − | | − | − | − | |
| 226 | − | − | − | | − | − | ++ | |
| 227 | − | − | ++ | | − | ++ | − | |
| 228 | ++ | − | − | Y | − | − | + | |
| 229 | − | − | − | | − | − | + | |
| 230 | − | − | + | | − | − | − | |
| 231 | − | − | − | | − | − | ++ | |
| 232 | − | − | − | | + | − | − | |
| 233 | − | − | − | | ++ | + | − | Y |
| 234 | + | ++ | − | Y | − | − | ++ | |
| 235 | − | − | − | | − | − | − | |
| 236 | − | − | − | | − | − | − | |
| 237 | − | − | − | | − | − | − | |
| 238 | − | − | − | | − | − | − | |
| 239 | − | − | + | | − | − | ++ | |
| 240 | + | − | − | | − | − | ++ | |
| 241 | − | − | + | | − | − | ++ | |
| 242 | − | − | − | | − | − | − | |
| 243 | − | − | + | | − | − | + | |
| 244 | + | − | − | | ++ | − | − | |
| 245 | − | − | ++ | Y | − | − | ++ | |
| 246 | − | − | − | | + | + | − | |
| 247 | − | − | − | | − | − | − | |
| 248 | − | + | − | | − | − | − | |
| 249 | − | + | − | | − | − | − | |
| 250 | − | − | − | | − | + | − | |
| 251 | ++ | − | − | | + | − | − | |
| 252 | − | − | − | | + | − | ++ | Y |
| 253 | − | − | − | | − | − | − | |
| 254 | − | − | − | | − | + | − | |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A bifunctional oligonucleotide comprising:
(i) an alternative splice site targeting sequence capable of binding an exonic element (e.g., 5′ splice site) within a target sequence (e.g., an RNA, e.g., a pre-mRNA or mRNA); and
(ii) a spliceosome targeting sequence capable of binding a spliceosome component (e.g., a U1 snRNP).

2. The bifunctional oligonucleotide of claim 1, wherein the bifunctional oligonucleotide is a single-stranded oligonucleotide.

3. The bifunctional oligonucleotide of claim 1, wherein the bifunctional oligonucleotide is an antisense oligonucleotide.

4. The bifunctional oligonucleotide of claim 1, wherein the bifunctional oligonucleotide is between 25 and 75 nucleotides in length (e.g, between 25 and 70 nucleotides, between 30 and 65 nucleotides, between 40 and 60 nucleotides).

5. The bifunctional oligonucleotide of claim 1, wherein the bifunctional oligonucleotide is between 5 and 50 nucleotides in length.

6. The bifunctional oligonucleotide of claim 1, wherein the sequence of (i) is between 5 and 35 nucleotides in length.

7. The bifunctional oligonucleotide of claim 1, wherein the sequence of (ii) is between 5 and 20 nucleotides in length.

8. The bifunctional oligonucleotide of claim 1, wherein the sequence of (i) is present at the 5′ terminus of the bifunctional oligonucleotide.

9. The bifunctional oligonucleotide of claim 1, wherein the sequence of (i) is present at the 3′ terminus of the bifunctional oligonucleotide.

10. The bifunctional oligonucleotide of claim 1, wherein the sequence of (ii) is present at the 5′ terminus of the bifunctional oligonucleotide.

11. The bifunctional oligonucleotide of claim 1, the sequence of (ii) is present at the 3′ terminus of the bifunctional oligonucleotide.

12. The bifunctional oligonucleotide of claim 1, comprising the structure of Formula (I): 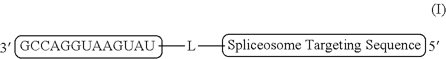
or a pharmaceutically acceptable salt thereof, wherein:
the alternative splice site targeting sequence comprises a nucleotide sequence capable of binding to a target sequence (e.g., an RNA, e.g., a pre-mRNA or mRNA) comprising an exonic element (e.g., an alternative splice site);
the spliceosome targeting sequence is a nucleotide sequence capable of binding to a spliceosome component (e.g., U1 snRNP), and
L is absent or a linker.

13. The bifunctional oligonucleotide of claim 1, comprising the structure of Formula (II): 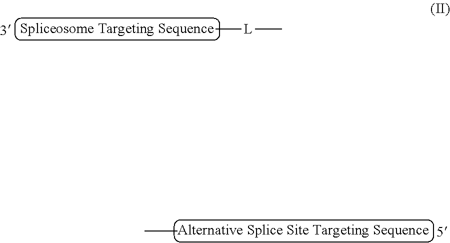
or a pharmaceutically acceptable salt thereof, wherein:
the alternative splice site targeting sequence comprises a nucleotide sequence capable of binding to a target sequence (e.g., an RNA, e.g., a pre-mRNA or mRNA) comprising an exonic element (e.g., an alternative splice site);
the spliceosome targeting sequence is a nucleotide sequence capable of binding to a spliceosome component (e.g., U1 snRNP), and
L is absent or a linker.

14. The bifunctional oligonucleotide of claim 1, 2 wherein the bifunctional oligonucleotide comprises a chemical modification (e.g., a non-naturally occurring chemical modification).

15. The bifunctional oligonucleotide of claim 14, wherein the chemical modification comprises a sugar modification, a nucleobase modification, a terminal modification, or an internucleotide linkage modification.

16. The bifunctional oligonucleotide of any of claims 14-15, wherein the chemical modification comprises a sugar modification (e.g, a 2′-ribose modification).

17. The bifunctional oligonucleotide of claim 16, wherein the sugar modification is a 2′-O-alkyl modification, a 2′-halo modification, or a 2′-deoxy modification.

18. The bifunctional oligonucleotide of any of claims 16-17, wherein the sugar modification comprises a 2′-OMe, 2′-MOE, 2′-H, 2′-Cl, 2′-F modification.

19. The bifunctional oligonucleotide of claim 14, wherein the chemical modification is a linked nucleic acid (LNA).

20. The bifunctional oligonucleotide of claim 14, wherein the chemical modification comprises a nucleobase modification (e.g., methylation).

21. The bifunctional oligonucleotide of claim 14, wherein the chemical modification comprises an internucleotide linkage modification (e.g., a phosphorothioate modification).

22. The bifunctional oligonucleotide of claim 4, wherein the bifunctional oligonucleotide comprises a plurality of chemical modifications.

23. The bifunctional oligonucleotide of claim 1, wherein the bifunctional oligonucleotide comprises a chemical modification within (i) and (ii).

24. The bifunctional oligonucleotide of claim 1, wherein the bifunctional oligonucleotide comprises a plurality of chemical modifications within (i) and (ii).

25. The bifunctional oligonucleotide of claim 1, wherein the bifunctional oligonucleotide comprises a plurality of sugar modifications or LNAs.

26. The bifunctional oligonucleotide of claim 1, wherein the bifunctional oligonucleotide comprises at least 1, 2, 3, 4, 5, 6, 7, 8, 9, 10, 15, 20, 25, 30, or more sugar modifications (e.g., 2′O-Me modifications).

27. The bifunctional oligonucleotide of claim 1, wherein the bifunctional oligonucleotide comprises at least 1, 2, 3, 4, 5, 6, 7, 8, 9, 10, 15, 20, 25, 30, or more LNAs.

28. The bifunctional oligonucleotide of claim 1, comprising the structure of Formula (I-b): 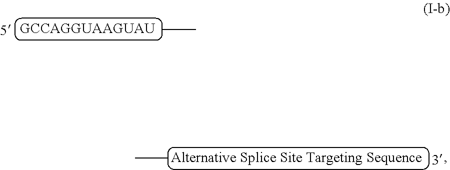
or a pharmaceutically acceptable salt thereof, wherein the alternative splice site targeting sequence is a sequence selected from: AAAAGCAGAACCUGAGCGGC, UUCCAGGGUCGCCATGGCGG, UCAGCUTUUCCAGGGUCGCC, AAGGACTUGAGGGACUCGAA, and AAGGACTUGAGGGACUCGAA; and each of bases 1-33 may be optionally modified with one or modifications selected from: 2′OMe modification, locked nucleic acid modification (LNA), 2′-O-methoxy ethyl modification, and phosphorothioate modification

29. The bifunctional oligonucleotide of claim 28, wherein the alternative splice site targeting sequence comprises AAAAGCAGAACCUGAGCGGC.

30. The bifunctional oligonucleotide of claim 28, wherein the alternative splice site targeting sequence comprises UUCCAGGGUCGCCATGGCGG.

31. The bifunctional oligonucleotide of claim 28, wherein the alternative splice site targeting sequence comprises UCAGCUTUUCCAGGGUCGCC.

32. The bifunctional oligonucleotide of claim 28, wherein the alternative splice site targeting sequence comprises AAGGACTUGAGGGACUCGAA.

33. The bifunctional oligonucleotide of claim 28, wherein the alternative splice site targeting sequence comprises UUCAUCAGCUTUUCCAGGGU.

34. The bifunctional oligonucleotide of claim 28, wherein bases 1-15, 17-19, 21-23, 25-27, 29-31, and 33 comprise a 2′OMe modification; bases 2, 3, 4, and 5 comprise a phosphorothioate modification; and bases 16, 20, 24, 28, and 32 comprise an LNA modification.

35. The bifunctional oligonucleotide of claim 28, wherein bases 1-15, 17-19, 21-23, 25-27, 29-31, and 33 comprise a 2′OMe modification; bases 30, 31, 32, and 33 comprise a phosphorothioate modification; and bases 16, 20, 24, 28, and 32 comprise an LNA modification.

36. The bifunctional oligonucleotide of claim 28, wherein bases 1-15, 17-19, 21-23, 25-27, 29-31, and 33 comprise a 2′OMe modification; bases 2, 3, 4, 5, 30, 31, 32, and 33 comprise a phosphorothioate modification; and bases 16, 20, 24, 28, and 32 comprise an LNA modification.

37. The bifunctional oligonucleotide of claim 28, wherein bases 1-13 comprise a 2′OMe modification; bases 14, 15, 17-19, 21-23, 25-27, 29-31, and 33 comprise a 2′-O-methoxy ethyl modification; bases 30, 31, 32, and 33 comprise a phosphorothioate modification; and bases 16, 20, 24, 28, and 32 comprise an LNA modification.

38. The bifunctional oligonucleotide of claim 28, wherein bases 1-13 comprise a 2′OMe modification; bases 14, 15, 17-19, 21-23, 25-27, 29-31, and 33 comprise a 2′-O-methoxy ethyl modification; bases 2, 3, 4, 5, 30, 31, 32, and 33 comprise a phosphorothioate modification; and
bases 16, 20, 24, 28, and 32 comprise an LNA modification.

39. The bifunctional oligonucleotide of claim 28, wherein bases 1, 3, 5, 7, 9, and 11-13 comprise 2′-O-methoxy ethyl modification; bases 14, 15, 17-19, 21-23, 25-27, 29-31, and 33 comprise a 2′OMe modification; bases 2, 3, 4, 5, 30, 31, 32, and 33 comprise a phosphorothioate modification; and bases 2, 4, 6, 8, 10, 16, 20, 24, 28, and 32 comprise an LNA modification.

40. The bifunctional oligonucleotide of claim 28, wherein bases 1, 3, 5, 7, 9, 11-13, 14, 15, 17-19, 21-23, 25-27, 29-31, and 33 comprise a 2′-O-methoxy ethyl modification; bases 2, 3, 4, 5, 30, 31, 32, and 33 comprise a phosphorothioate modification; and bases 2, 4, 6, 8, 10, 16, 20, 24, 28, and 32 comprise an LNA modification.

41. The bifunctional oligonucleotide of claim 28, wherein bases 1, 3, 5, 7, 9, and 11-13 comprise 2′-O-methoxy ethyl modification; bases 14, 15, 17-19, 21-23, 25-27, 29-31, and 33 comprise a 2′OMe modification; bases 2, 3, 4, and 5 comprise a phosphorothioate modification; and bases 2, 4, 6, 8, 10, 16, 20, 24, 28, and 32 comprise an LNA modification.

42. The bifunctional oligonucleotide of claim 1, wherein the alternative splice site (e.g., 5′ splice site) is present within a gene containing a nucleotide repeat expansion.

43. The bifunctional oligonucleotide of claim 1, wherein the alternative splice site (e.g., 5′ splice site) is present within a gene containing a trinucleotide repeat expansion.

44. The bifunctional oligonucleotide of claim 1, wherein the alternative splice site (e.g., 5′ splice site) is present within any one of the genes listed in Table 3.

45. The bifunctional oligonucleotide of claim 1, wherein the alternative splice site (e.g., 5′ splice site) is present within the HTT gene.

46. The bifunctional oligonucleotide of claim 1, wherein the alternative splice site is present within exon 1 of the HTT gene.

47. The bifunctional oligonucleotide of claim 1, wherein the alternative splice site within exon 1 is upstream of a CAG region within exon 1.

48. The bifunctional oligonucleotide of claim 1, wherein the alternative splice site comprises the sequence GAGT (SEQ ID NO: 002) or AAGT (SEQ ID NO: 003).

49. The bifunctional oligonucleotide of any of the preceding claims, wherein the U1 snRNP is a wild type U1 snRNP or a variant or fragment thereof.

50. The bifunctional oligonucleotide of claim 1, wherein the bifunctional oligonucleotide comprises a sequence with at least 75%, 80%, 85%, 90%, 95%, 99%, or more sequence identity with SEQ ID NO: 004, or a variant or fragment thereof.

51. The bifunctional oligonucleotide of claim 1, wherein the sequence of (ii) comprises a sequence with at least 75%, 80%, 85%, 90%, 95%, 99%, or more sequence identity to SEQ ID NO: 004.

52. The bifunctional oligonucleotide of claim 1, wherein the sequence of (ii) comprises SEQ ID NO: 004.

53. The bifunctional oligonucleotide of claim 1, wherein the sequence of the bifunctional oligonucleotide is at least 75%, 80%, 85%, 90%, 95%, 99%, or more sequence identity with an oligonucleotide listed in Table 1 or 2, e.g., an oligonucleotide selected from SEQ ID NOs: 100-254, or a variant or fragment thereof.

54. The bifunctional oligonucleotide of claim 1, wherein the sequence of (i) comprises a sequence with at least 75%, 80%, 85%, 90%, 95%, 99%, or more sequence identity with a nucleotide selected from SEQ ID NOs: 100-254, or a variant or fragment thereof.

55. The bifunctional oligonucleotide of claim 1, wherein the sequence of (i) comprises a sequence selected from SEQ ID NOs: 100-254, or a variant or fragment thereof.

56. A bifunctional oligonucleotide comprising:
(i) a nucleotide sequence capable of binding to a splice site (e.g., 5′ splice site) within exon 1 of the HTT gene, wherein the nucleotide sequence comprises a plurality of chemical modifications (e.g., a plurality of 2′OMe modifications and LNA modifications) and is between 5 and 35 nucleotides in length; and
(b) a nucleotide sequence capable of binding an U1 snRNA comprising a plurality of chemical modifications (e.g., a plurality of 2′OMe modifications).

57. The bifunctional oligonucleotide of claim 1 or 56, wherein the bifunctional oligonucleotide is capable of one or more of:
(a) enhancing exonization of the HTT gene (e.g., exon 1) by recruiting the U1 snRNP to an alternative 5′ splice site;
(b) altering the sequence of the HTT gene (e.g., exon 1) by recruiting the U1 snRNP to an alternative 5′ splice site; and
(b) potentiating U1 usage/recruitment.

58. The bifunctional oligonucleotide of claim 57, comprising (a).

59. The bifunctional oligonucleotide of claim 57, comprising (b).

60. A pharmaceutical composition comprising a bifunctional oligonucleotide of any of claims 1-59.

61. The bifunctional oligonucleotide of any of claims 1-59, disposed in a delivery vehicle.

62. The bifunctional oligonucleotide of claim 61, wherein the delivery vehicle is a membrane-bound delivery vehicle.

63. The bifunctional oligonucleotide of claim 61, wherein the delivery vehicle comprises a liposome or lipid nanoparticle.

64. A method of modulating the production or level of a transcription product in a cell or subject comprising an exonic element (e.g., a trinucleotide expansion, e.g., a [CAG]ₙ site) in a subject or cell, wherein:
(i) the exonic element is flanked by a proximal splice site and a distal splice site, and
(ii) the proximal splice site and distal splice sites are both 5′ splice sites or are both 3′ splice sites;
comprising contacting said cell or subject with a bifunctional oligonucleotide promoting splicing at:
(a) the distal 5′ splice site to (a-i) decrease the production or level of a transcription product comprising the exonic element or (a-ii) increase the production or level of a transcription product lacking the exonic element; or
(b) the proximal 3′ splice site to (b-i) increase the production or level of a transcription product comprising the exonic element or (b-ii) decrease the production or level of a transcription product lacking the exonic element;
thereby modulating the production or level of a transcription product comprising an exonic element (e.g., a trinucleotide expansion, e.g., a [CAG], site).

65. The method of claim 64, comprising (a-i) or (a-ii).

66. The method of claim 64, comprising (a-i).

67. The method of claim 64, comprising (b-i) or (b-ii).

68. The method of claim 64, comprising (b-i).

69. The method of any of claims 64-68, wherein, the distal 5′ splice site is a non-canonical 5′ splice site.

70. The method of any of claims 64-69, wherein the proximal 5′ splice site is a canonical 5′ splice site.

71. The method of any of claims 64-70, wherein the distal 5′ splice site is a non-canonical 5′ splice site and the exonic element comprises a canonical 5′ spice site.

72. The method of any of claims 64-71, wherein the exonic element comprises a trinucleotide repeat.

73. The method of claim 72, wherein the trinucleotide repeat comprises the nucleotide sequence CXY, wherein each of X and Y is selected from A, T, G, and C.

74. The method of claim 72, wherein the trinucleotide repeat is selected from CAG, CGG, or CGG.

75. The method of claim 64, wherein the alternative splice site is the 5′ distal splice site.

76. The method of claim 64, wherein the production or level of a transcription product produced by splicing at the distal 5′ splice site is increased by at least 5%, 10%, 20%, 30%, 40%, 50%, 60%, 70%, 80%, or more, e.g., in comparison to a reference standard (e.g., the transcription product produced by splicing at proximal 5′ splice site, wild type transcription product, mutant transcription product, etc).

77. The method of claim 64, wherein the production or level of a transcription product produced by splicing at the proximal 5′ splice site is decreased by at least 5%, 10%, 20%, 30%, 40%, 50%, 60%, 70%, 80%, or more, e.g., in comparison to a reference standard (e.g., the transcription product produced by splicing at distal 5′ splice site, wild type transcription product, mutant transcription product, etc).

78. The method of claim 64, wherein the exonic element is flanked by a plurality of distal splice sites, e.g., distal 5′ splice sites.

79. The method of claim 64, wherein the exonic element is flanked by a plurality of proximal splice sites, e.g., proximal 5′ splice sites.

80. The method of any one of claims 64-79, wherein the ratio of:
(A) the production or level of a transcription product arising from splicing at the 5′ distal splice site (e.g., a less-favored, less efficient, or non-canonical splice site) to
(B) the production or level of a transcription product arising from splicing at the 5′ proximal splice site (e.g., a more-favored, more efficient, or a canonical splice site),
is between 1:1-10:1, or 1:1-1:10.

81. The method of claim 80, wherein the ratio of (A) to (B) is decreased by the modulating.

82. The method of claim 80, wherein the decrease of the ratio of (A) to (B) is about 5%, 10% 15%, 20% 25%, 30%, 35%, 40%, 45%, 50%, or more.

83. The method of claim 80, wherein the ratio of (A) to (B) is lower at one allele or chromosome (e.g., a mutant allele, e.g., an allele comprising a tri-nucleotide repeat) than at the other allele or chromosome (e.g., a non-mutant allele, e.g., an allele not comprising a tri-nucleotide repeat).

84. The method of claim 80, wherein the bifunctional oligonucleotide is a bifunctional oligonucleotide of any one of claims 1-45.

85. The method of claim 80, wherein the production or level of a transcription product produced by splicing at the distal 5′ splice site is increased by at least 5%, 10%, 20%, 30%, 40%, 50%, 60%, 70%, 80%, e.g., in comparison to a cell or subject not administered the bifunctional oligonucleotide.

86. The method of any one of claims 80-85, wherein the production or level of a transcription product produced by splicing at the proximal 5′ splice site is decreased by at least 5%, 10%, 20%, 30%, 40%, 50%, 60%, 70%, 80%, e.g., in comparison to a cell or subject not administered the bifunctional oligonucleotide.

87. The method of any one of claims 80-86, wherein the bifunctional oligonucleotide has one or more of the following properties:
(i) is capable of binding at or near to a distal 5′ splice site or a distal 3′ splice site;
(ii) is capable of binding at or near to a U1 snRNP (e.g., a mutant U1 snRNP); and
(iii) is capable of promoting splicing of a mutant pre-mRNA relative to a wild-type pre-mRNA.

88. The method of any one of claims 80-87, wherein the exonic element comprises the nucleotide sequence [CAG]ₙ, wherein n is an integer between 10 and 60.

89. The method of claim 80, wherein the nucleotide sequence [CAG]ₙ forms a double-stranded structure (e.g., hairpin structure).

90. The method of claim 80, wherein the modulating is dependent on the length of the nucleotide sequence [CAG]ₙ.

91. The method of claim 80, wherein the transcription product is an HTT mRNA.

92. The method of claim 80, wherein the exonic element is present within an exon of the HTT gene.

93. The method of claim 80, wherein the exonic element is present within exon 1 of the HTT gene.

94. The method of claim 80, wherein the distal 5′ splice site is present within exon 1 of the HTT gene.

95. The method of any one of claims 50-80, wherein the proximal splice site and the distal splice site are 3′splice sites.

96. The method of claim 81, comprising promoting splicing at the distal 3′ splice site to decrease the production or level of a transcription product comprising the exonic element.

97. The method of any one of claims 80-96, comprising promoting splicing at the distal 3′ splice site to increase the production or level of a transcription product that lacks the exonic element.

98. The method of any one of claims 80-97, wherein the distal splice site is a non-canonical 3′ splice site.

99. The method of any one of claims 80-98, wherein the proximal splice site is a canonical 3′ splice site.

100. The method of any one of claims 80-99, wherein the distal splice site is a non-canonical 3′ splice site and the gene comprises a canonical 3′ splice site disposed between the exonic element and cognate intron of the canonical 3′ splice site.

101. The method of claim 100, comprising promoting splicing at the proximal splice site to increase the production of a transcription product comprising the exonic element.

102. The method of claim 100, comprising promoting splicing at the proximal splice site to decrease the production or level of a transcription product lacking the exonic element.

103. The method of any of claims 80-102, wherein a first and second allele of the gene are present.

104. The method of claim 103, wherein the first allele is carried on a first chromosome and the second allele is carried on a second chromosome.

105. The method of any of claims 89-90, wherein the sequence of the exonic element of one allele differs from the sequence of the exonic element on the second allele.

106. The method of any of claims 89-91, wherein one allele comprises the exonic element and the other allele does not comprise the exonic element.

107. The method of any of claims 89-92, wherein
(i) a transcription product comprising a first form of the exonic element, e.g., a [CAG]ₙ, is transcribed from an allele on a first chromosome; and
(ii) a transcription product which lacks the first form of the exonic element, is transcribed from an allele on a second chromosome.

108. The method of any of claims 89-93, where one of the alleles on the first and second chromosome is wild type for the gene which is not disease-associated and the other is mutant or disease associated allele for the gene.

109. The method of claim 108, performed in vitro.

110. The method of claim 108, performed in vivo.

111. The method of any of claims 108-110, wherein splicing of the distal splice site is more efficient than is splicing of the proximal splice site.

112. A method of modulating splicing at an alternative splice site (e.g., a flanking an exonic element, e.g., a trinucleotide expansion, e.g., a [CAG],I site) wherein:
(i) the exonic element is flanked by a proximal splice site a distal splice site, and
(ii) the proximal splice site and distal splice sites are both 5′ splice sites or are both 3′ splice sites;
comprising
(a) (i) wherein the alternative splice site is the 5′ distal splice site, promoting splicing at the 5′ distal splice site, or
(b) (i) wherein the alternative splice site is the 3′ distal splice site, promoting splicing at the proximal 3′splice site;
thereby modulating splicing at an alternative splice site.

113. The method of claim 112, wherein the exonic element comprises a trinucleotide repeat.

114. The method of claim 112, wherein the trinucleotide repeat comprises the nucleotide sequence CXY, wherein each of X and Y is selected from A, T, G, and C.

115. The method of claim 112, wherein the trinucleotide repeat is selected from CAG, CGG, or CGG.

116. The method of any one of claims 112-115, wherein the alternative splice site is the 5′ distal splice site.

117. The method of any one of claims 112-116, wherein the alternative splice site is the 3′ distal splice site.

118. A method of modulating splicing at an alternative splice site within the HTT gene in a subject or cell, wherein the HTT gene comprises a [CAG]ₙ site and the splice site is present upstream of the [CAG]ₙ site.

119. The method of claim 118, comprising administering to the subject or cell an exogenous modulator.

120. A method of treating a trinucleotide repeat expansion disease comprising modulating splicing at an alternative splice site in a subject, wherein the alternative splice site is flanking an exonic element (e.g., a trinucleotide expansion, e.g., a [CAG]ₙ site) wherein:
(i) the exonic element is flanked by a proximal splice site a distal splice site, and
(ii) the proximal splice site and distal splice sites are both 5′ splice sites or are both 3′ splice sites;
comprising
(a) (i) wherein the alternative splice site is the 5′ distal splice site, promoting splicing at the 5′ distal splice site, or
(b) (i) wherein the alternative splice site is the 3′ distal splice site, promoting splicing at the proximal 3′splice site;
thereby treating a trinucleotide repeat expansion disease.

121. A method of treating a trinucleotide repeat expansion disease, comprising administering to a subject a bifunctional oligonucleotide of any one of claims 1-120, or a composition thereof.

121. A composition for use in treating a trinucleotide repeat expansion disease, comprising a bifunctional oligonucleotide of any one of claims 1-120.
